(19) United States
(12) Patent Application Publication
(10) Pub. No.: US 2025/0268884 A1
(43) Pub. Date: Aug. 28, 2025

(54) Deuterated Domperidone for Treating Gastroparesis

(71) Applicant: CinDome Pharma, Inc., Cincinnati, OH (US)

(72) Inventors: Mary BOND, Cincinnati, OH (US); Brendan DORAN, Cincinnati, OH (US); Brian MURPHY, Cincinnati, OH (US); Jon ISAACSOHN, Cincinnati, OH (US)

(21) Appl. No.: 18/857,909

(22) PCT Filed: Apr. 20, 2023
(86) PCT No.: PCT/US2023/019319

§ 371 Date: Oct. 18, 2024

Related U.S. Application Data
(60) Provisional application No. 63/333,034, filed on Apr. 20, 2022.
(60) Provisional application No. 63/333,042, filed on Apr. 20, 2022.

(51) Int. Cl. A61K 31/454; A61P 1/00

(52) U.S. Cl. CPC A61K 31/454; A61P 1/00

(57) ABSTRACT

The disclosure relates to methods for increasing gastric emptying in a human having gastroparesis.

### CROSS-REFERENCE TO RELATED APPLICATIONS

This application claims the benefit of U.S. Provisional Patent Application Nos. 63/333,042, filed Apr. 20, 2022, and 63/333,034, filed Apr. 20, 2022, the disclosures of which are incorporated by reference herein.

### TECHNICAL FIELD

The disclosure relates to methods for increasing gastric emptying in a human having gastroparesis.

### BACKGROUND

Gastroparesis is defined as impaired gastric emptying in the absence of physical gastric outlet obstruction. Symptoms include early satiety, postprandial fullness, nausea, vomiting, and abdominal pain. Gastroparesis affects the patient's nutritional state, especially in diabetics, and severe symptoms of gastroparesis can lead to other complications such as malnutrition, esophagitis, and Mallory-Weiss tears. Gastroparesis negatively impacts a patient's quality of life, causing decreased social interaction, poor work functionality, and development of anxiety or depression.

The first-line treatment for gastroparesis involves nutritional management and support, including fluid and electrolyte replacement. Currently, metoclopramide, a dopamine D₂ receptor antagonist, is the only United States Food and Drug Administration-approved agent for the treatment of gastroparesis. Unfortunately, this agent readily crosses the blood-brain barrier resulting in central nervous system (CNS) side effects in up to 40% of patients.

Safe and effective treatments for gastroparesis are needed.

### SUMMARY

In some aspects, the disclosure provides methods for increasing gastric emptying in a human having gastroparesis, comprising orally administering 5 mg to 120 mg of deudomperidone daily to the human.

Other aspects and embodiments of the invention will be readily apparent from the following detailed description of the invention.

### BRIEF DESCRIPTION OF THE DRAWINGS

FIG. 1 is the study schematic for Example 2. In this figure, * Anticipated dose level: ** For each cohort, subjects were randomized to CIN-102 or placebo. Randomization occurred on the morning of Day 1: *** Subjects participating in the intensive PK subgroup were seen in the clinic on Day 15 and Day 16 (+1 day). Subjects participating in the sparse PK subgroup were seen on Day 15 OR Day 16 (+1 day).

FIGS. 2A and 2B are plots of mean (±standard deviation) plasma deudomperidone concentrations by treatment following the morning dose of deudomperidone on Days 1 and 14 on a linear scale for the pharmacokinetic population. FIG. 2A is for Cohort 3 (5 mg deudomperidone BID) and FIG. 2B is for Cohort 1 (10 mg deudomperidone BID). In this figure, LLOQ of deuterated domperidone=0.01 ng/ml. Concentrations outside of the respective time point's analysis collection window were excluded from this plot.

FIG. 3 are plots of mean (±Standard Deviation) plasma deuterated domperidone concentrations by treatment on Days 1 and 14 on linear and semi-logarithmic scales for the pharmacokinetic population-Cohort 2: CIN-102 20 mg QD. For this figure. LLOQ of deuterated domperidone=0.01 ng/ml. Concentrations outside of the respective time point's analysis collection window were excluded from this plot. No 24-hour time point was collected on Day 1.

FIG. 4 is a line graph of change-from-baseline HR (ΔHR) across time points (QT/QTc population). In this figure, LS mean and 90% CI based on a linear mixed-effects model for active and placebo treatment groups: ΔHR=Time+Treatment+Time×Treatment+Baseline HR+Period+Sequence. An unstructured covariance structure was used to specify the repeated measures at post-dose time points for subject within treatment period. The model also included a subject-specific random effect.

FIG. 5 is a line graph of the change-from-baseline QTcF (ΔQTcF) across time points (QT/QTc population). In this figure, LS mean and 90% CI based on a linear mixed-effects model for active and placebo treatment groups: ΔQTcF=Time+Treatment+Time×Treatment+Baseline QTcF-Period+Sequence. An unstructured covariance structure was used to specify the repeated measures at post-dose time points for subject within treatment period. The model also included a subject-specific random effect.

FIG. 6 is a scatter plot of the observed M3 plasma concentrations and estimated placebo-adjusted ΔQTcF (PK/QTc population). In this figure, the solid red line with dashed red lines denotes the model-predicted mean ΔΔQTcF with 90% CI, which is calculated from the equation ΔΔQTcF=0.3678 (ms)+0.1439 (ms per ng/ml)× M3 concentration (ng/mL). The plotted points denote the pairs of observed drug plasma concentrations and estimated placebo-adjusted ΔQTcF (ΔΔQTcF) by subjects for each active treatment group and placebo group. The individually estimated placebo-adjusted ΔQTcFi,k (ΔΔQTcFi,k) equals the individual ΔQTcFi,k for subject i administered with active drug or placebo at time point k minus the estimation of the time effect at time point k.

FIG. 7 is a graph of model-predicted ΔΔQTcF (mean and 90% CI) and estimated placebo-adjusted ΔQTcF (mean and 90% CI) across deciles of M3 plasma concentrations (PK/QTc population). In this figure, the solid black line with gray shaded area denotes the model-predicted mean ΔΔQTcF with 90% CI, which is calculated from the equation ΔΔQTcF=0.3678 (ms)+0.1439 (ms per ng/ml)×M3 concentration (ng/ml). The red filled circles with vertical bars denote the estimated mean placebo-adjusted ΔQTcF (ΔΔQTcF) with 90% CI displayed at the associated median plasma concentration within each decile for M3 concentration, among which the individually estimated placebo-adjusted ΔQTcFi,k (ΔΔQTcFi,k) equals the individual ΔQTcFi,k for subject i administered with M3 at time point k minus the estimation of time effect at time point k. The black circle with vertical bars denotes the mean placebo-adjusted ΔQTcF with 90% CI for placebo at a concentration of 0. The horizontal red line with notches shows the range of concentrations divided into deciles for M3 concentration. The area between each decile represents the point at which 10% of the data are present: the first notch to second notch denotes the first 10% of the data, the second notch to third notch denotes the 10-20% of the data and so on.

FIG. 8 is a graph of the model-predicted ΔΔQTcF interval across the range of observed deudomperidone plasma concentrations and at geometric mean peak deudomperidone concentrations following administration of 30 mg and 100 mg CIN-102 (PK/QTc population). In this figure, the solid black line with gray shaded area denotes the model-predicted mean (90% CI) ΔΔQTcF, which is calculated from the equation ΔΔQTcF=−0.02844 (ms)+0.7918 (ms per ng/ml)× deudomperidone concentration (ng/ml). The vertical blue and red lines denote the estimated mean (90% CI) ΔΔQTcF with plotted points at the geometric mean Cₘₐₓ of deudomperidone.

FIG. 9 is a graph of placebo-corrected change-from-baseline HR (ΔΔHR) across time points (QT/QTc population). LS mean and 90% CI based on a linear mixed-effects model for active and placebo treatment groups: ΔHR=Time+Treatment+Time×Treatment+Baseline HR+Period+Sequence. An unstructured covariance structure was used to specify the repeated measures at post-dose time points for subject within treatment period. The model also included a subject-specific random effect.

FIG. 10 is a Scatter plot of observed deuterated domperidone plasma concentrations and ΔQTcF with simple linear regression line and LOESS regression (PK/QTc populations). In this figure. The red line with the blue shaded area denotes the LOESS regression and 90% confidence limits. The black solid line denotes the simple linear regression line. The plotted points denote the pairs of observed Deuterated Domperidone plasma concentrations and ΔQTcF.

FIG. 11 is a graph of the placebo-corrected change-from-baseline QTcF (ΔΔQTcF) across time points (QT/QTc population). LS mean and 90% CI based on a linear mixed-effects model for active and placebo treatment groups: ΔQTcF=Time+Treatment+Time×Treatment+Baseline QTcF+Period+Sequence. An unstructured covariance structure was used to specify the repeated measures at post-dose time points for subject within treatment period. The model also included a subject-specific random effect.

FIG. 12 is a graph of the mean deuterated domperidone plasma concentrations over time by dose (PK/QTc population). This figure displays mean±SD from descriptive statistics. If mean-SD was below 0, 0 was substituted as concentrations below 0 are not biologically plausible.

FIG. 13 is a graph of the mean CIN-102 M3 plasma concentrations over time by dose (PK/QTc population). This figure displays mean±SD from descriptive statistics. If mean−SD was below 0, 0 was substituted as concentrations below 0 are not biologically plausible.

FIG. 14 is a scatter plot of observed CIN-102 M3 plasma concentrations and ΔQTcF with simple linear regression line and LOESS regression (PK/QTc population). The red line with the blue shaded area denotes the LOESS regression and 90% confidence limits. The black solid line denotes the simple linear regression line. The plotted points denote the pairs of observed CIN-102 M3 plasma concentrations and ΔQTcF.

FIG. 15 is a scatter plot of observed deuterated domperidone plasma concentrations and estimated placebo-adjusted ΔQTcF (PK/QTc Population). The solid red line with dashed red lines denotes the model-predicted mean ΔΔQTcF with 90% CI, which is calculated from the equation ΔΔQTcF=−0.02844 (ms)+0.7918 (ms per ng/ml)× deuterated domperidone concentration (ng/ml). The plotted points denote the pairs of observed drug plasma concentrations and estimated placebo-adjusted ΔQTcF (ΔΔQTcF) by subjects for each active dose group and placebo group. The individually estimated placebo-adjusted ΔQTcF_{i,k} (ΔΔQTcF_{i,k}) equals the individual ΔQTcF_{i,k} for subject i administered with active drug or placebo at time point k minus the estimation of the time effect at time point k.

FIG. 16 is a graph of the model-predicted ΔΔQTcF (mean and 90% CI) and estimated placebo-adjusted ΔQTcF (mean and 90% CI). across deciles of deuterated domperidone plasma concentrations (PK/QTc population). The solid black line with gray shaded area denotes the model-predicted mean ΔΔQTcF with 90% CI, which is calculated from the equation ΔΔQTcF=−0.02844 (ms)+0.7918 (ms per ng/ml)× deuterated domperidone concentration (ng/ml). The red filled circles with vertical bars denote the estimated mean placebo-adjusted ΔQTcF (ΔΔQTcF) with 90% CI displayed at the associated median plasma concentration within each decile for deuterated domperidone concentration, among which the individually estimated placebo-adjusted ΔQTcF_{i,k} (ΔΔQTcF_{i,k}) equals the individual ΔQTcF_{i,k} for subject i administered with deuterated domperidone at time point k minus the estimation of time effect at time point k. The black circle with vertical bars denotes the mean placebo-adjusted ΔQTcF with 90% CI for placebo at a concentration of 0. The horizontal red line with notches shows the range of concentrations divided into deciles for deuterated domperidone concentration. The area between each decile represents the point at which 10% of the data are present: the first notch to second notch denotes the first 10% of the data, the second notch to third notch denotes the 10-20% of the data and so on.

FIG. 17 is a graph of the change-from-baseline PR (ΔPR) across time points (QT/QTc population). LS mean and 90% CI based on a linear mixed-effects model for active and placebo treatment groups: ΔPR=Time+Treatment+Time×Treatment+Baseline PR+Period+Sequence. An unstructured covariance structure was used to specify the repeated measures at post-dose time points for subject within treatment period. The model also included a subject-specific random effect.

FIG. 18 is a graph of the placebo-corrected Change-from-baseline PR (ΔΔPR) across time points (QT/QTc population). LS mean and 90% CI based on a linear mixed-effects model for active and placebo treatment groups: ΔPR=Time+Treatment+Time×Treatment+Baseline PR+Period+Sequence. An unstructured covariance structure was used to specify the repeated measures at post-dose time points for subject within treatment period. The model also included a subject-specific random effect

FIG. 19 is a graph of the change-from-baseline QRS (ΔQRS) across time points (QT/QTc population). LS mean and 90% CI based on a linear mixed-effects model for active and placebo treatment groups: ΔQRS=Time+Treatment+Time×Treatment+Baseline QRS+Period+Sequence. An unstructured covariance structure was used to specify the repeated measures at post-dose time points for subject within treatment period. The model also included a subject-specific random effect.

FIG. 20 is a graph of the placebo-corrected Change-from-baseline QRS (ΔΔQRS) across time points (QT/QTc population). LS mean and 90% CI based on a linear mixed-effects model for active and placebo treatment groups: ΔQRS=Time+Treatment+Time×Treatment+Baseline QRS+Period+Sequence. An unstructured covariance structure was used to specify the repeated measures at post-dose time points for subject within treatment period. The model also included a subject-specific random effect.

FIG. 21 is a graph of the Model-predicted ΔΔQTcF interval at geometric mean peak at geometric mean peak CIN-102 M3 concentrations (PK/QTc population). In this figure, The solid black line with gray shaded area denotes the model-predicted mean (90% CI) ΔΔQTcF, which is calculated from the equation ΔΔQTcF=0.3678 (ms)+0.1439 (ms per ng/mL)×CIN-102 M3 concentration (ng/mL). The non-gray shaded areas denote the estimated mean (90% CI) ΔΔQTcF with plotted points at the geometric mean Cmax of CIN-102 M3.

### DETAILED DESCRIPTION OF ILLUSTRATIVE EMBODIMENTS

In the disclosure, the singular forms “a,”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude an optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

The terms “subject” and “patient” are used interchangeably and typically refer to a human. In some embodiments, the human is an adult, i.e., 18 years of age or older. In other embodiments, the human is a child, i.e., younger than 18 years of age.

“Treating” or variations thereof refers to eliminating or reducing at least one physical parameter of the disease or disorder. In other embodiments, “treating” refers to modulating the disease or disorder, either physically, (e.g., by stabilizing a discernible symptom), physiologically, (e.g., stabilizing a physical parameter), or both. In further embodiments, “treating” refers to delaying onset of the disease or disorder.

“Deudomperidone,” “d₄-domperidone,” or “CIN-102” as referenced herein are interchangeable and refer to 1-{3-[4-(5-chloro-2-oxo-2,3-dihydro-1H-1,3-benzodiazol-1-yl)piperidin-1yl]propyl}-2,3-dihydro(4,5,6,7-D₄)-1H-1,3-benzodiazol-2-one, which has the following structure:

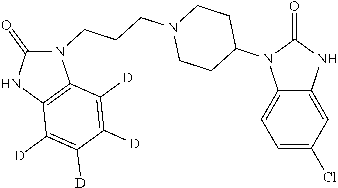 [SMILES: [2H]c1c([2H])c([2H])c2c([nH]c(=O)n2CCCN2CCC(n3c(=O)[nH]c4cc(Cl)ccc43)CC2)c1[2H]]

Any reference to deudomperidone may also include, where noted, pharmaceutically acceptable salts, esters, hydrates, solvates, prodrug forms, and derivatives of these, which are broadly defined as deudomperidone compounds that are modified or partially substituted.

“Pharmaceutically acceptable” refers to properties and/or substances that are acceptable to the patient from a pharmacological/toxicological vantage, and to the manufacturing pharmaceutical chemist from a physical/chemical vantage regarding composition, formulation, stability, patient acceptance, and bioavailability.

A pharmaceutically acceptable salt includes salts with a pharmaceutically acceptable acid or base. In some embodiments, the pharmaceutically acceptable salt is with an acid such as an inorganic acid. Examples of inorganic acids include hydrochloric, sulfuric, phosphoric, diphosphoric, hydrobromic, hydroiodic and nitric acid. In other embodiments, the pharmaceutically acceptable salt is with an organic acid. Examples of organic acids include as citric, fumaric, maleic, malic, mandelic, ascorbic, oxalic, succinic, tartaric, benzoic, acetic, methanesulphonic, ethanesulphonic, benzenesulphonic, cyclohexylsulfamic (cyclamic) or p-toluenesulphonic acid. In further embodiments, the pharmaceutically acceptable salt is with a base such as an alkali metal. Examples of alkali metals include sodium or potassium. In yet other embodiments, the pharmaceutically acceptable salt is with a base such as an alkaline earth metal. Examples of alkaline earth metals include calcium or magnesium hydroxides. In still further embodiments, the pharmaceutically acceptable salt is with an organic base. Examples of organic bases include alkyl amines, arylalkyl amines and heterocyclic amines.

The abbreviation “D,” as used herein, refers to a stable isotope of hydrogen that is deuterium (heavy hydrogen or ²H). Such instances of “D” include an amount of deuterium that is above the naturally occurring distribution of deuterium. In some embodiments, D has deuterium enrichment of no less than about 1%. In other embodiments, D has a deuterium enrichment of no less than about 5%. In further embodiments, D has a deuterium enrichment of no less than about 10%. In still other embodiments, D has a deuterium enrichment of no less than about 20%. In still other embodiments, D has a deuterium enrichment of no less than about 30%. In still other embodiments, D has a deuterium enrichment of no less than about 40%. In yet further embodiments, D has a deuterium enrichment of no less than about 50%. In still other embodiments, D has a deuterium enrichment of no less than about 60%. In other embodiments, D has a deuterium enrichment of no less than about 70%. In further embodiments, D has a deuterium enrichment of no less than about 80%. In yet other embodiments, D has a deuterium enrichment of no less than about 90%. In still further embodiments, D has a deuterium enrichment of no less than about 98% of deuterium. In still further embodiments, D has a deuterium enrichment of no less than about 99% of deuterium. In still further embodiments, D has a deuterium enrichment of at least 99% of deuterium.

The disclosure provides methods for increasing gastric emptying, in particular, increasing gastric emptying in a human having gastroparesis. The term “gastroparesis” as used herein refers to a disorder involving delayed gastric emptying in the absence of physical gastric outlet obstruction. In some embodiments, the gastroparesis is idiopathic gastroparesis. In other embodiments, the gastroparesis is diabetic gastroparesis.

Prior to administration of deudomperidone, the human may be experiencing an upper gastrointestinal symptom. In some embodiments, the upper gastrointestinal symptom is nausea, vomiting, postprandial fullness, early satiety, bloating, epigastric pain, abdominal pain, or combinations thereof. In other embodiments, the upper gastrointestinal symptom is nausea. In further embodiments, the upper gastrointestinal symptom is vomiting. In yet other embodiments, the gastrointestinal symptom is postprandial fullness. In still further embodiments, the gastrointestinal symptom is early satiety. In other embodiments, the gastrointestinal symptom is bloating. In further embodiments, the gastrointestinal symptom is epigastric pain. In yet other embodiments, the gastrointestinal symptom is abdominal pain.

The methods include orally administering 5 mg to 120 mg of deudomperidone daily to the human. For example, the free base of deudomperidone is administered to the human. In some embodiments, the daily dose of deudomperidone is about 5, about 10, about 20, about 30, about 40, about 50, about 60, about 70, about 80, about 90, about 100, about 110, or about 120 mg. In other embodiments, the daily dose of deudomperidone about 5 to about 120, about 5 to about 110, about 5 to about 100, about 5 to about 90, about 5 to about 80, about 5 to about 70, about 5 to about 60, about 5 to about 50, about 5 to about 40, about 5 to about 30, about 5 to about 20, about 5 to about 10, about 10 to about 120, about 10 to about 110, about 10 to about 100, about 10 to about 90, about 10 to about 80, about 10 to about 70, about 10 to about 60, about 10 to about 50, about 10 to about 40, about 10 to about 30, about 10 to about 20, about 20 to about 120, about 20 to about 110, about 20 to about 100, about 20 to about 90, about 20 to about 80, about 20 to about 70, about 20 to about 60, about 20 to about 50, about 20 to about 40, about 20 to about 30, about 30 to about 120, about 30 to about 110, about 30 to about 100, about 30 to about 90, about 30 to about 80, about 30 to about 70, about 30 to about 60, about 30 to about 50, about 30 to about 40, about 40 to about 120, about 40 to about 110, about 40 to about 100, about 40 to about 90, about 40 to about 80, about 40 to about 70, about 40 to about 60, about 40 to about 50, about 50 to about 120, about 50 to about 110, about 50 to about 100, about 50 to about 90, about 50 to about 80, about 50 to about 70, about 50 to about 60, about 60 to about 120, about 60 to about 110, about 60 to about 100, about 60 to about 90, about 60 to about 80, about 60 to about 70, about 70 to about 120, about 70 to about 110, about 70 to about 100, about 70 to about 90, about 70 to about 80, about 80 to about 120, about 80 to about 110, about 80 to about 100, about 80 to about 90, about 90 to about 120, about 90 to about 110, about 90 to about 100, about 100 to about 120, about 100 to about 110, or about 110 to about 120 mg. In further embodiments, the daily dose of deudomperidone is about 5 mg. In yet other embodiments, the daily dose of deudomperidone is about 10 mg. In still further embodiments, the daily dose of deudomperidone is about 20 mg. In other embodiments, the daily dose of deudomperidone is about 30 mg. In further embodiments, the daily dose of deudomperidone is about 40 mg. In other embodiments, the daily dose of deudomperidone is about 60 mg. In yet other embodiments, the daily dose of deudomperidone is about 90 mg. In further embodiments, the daily dose of deudomperidone is about 120 mg.

Deudomperidone may be administered as needed to attain the desired daily dosage. For example, the deudomperidone can be administered in divided doses. In some aspects, deudomperidone is administered in one dose. In other aspects, deudomperidone is administered in two doses. In further aspects, deudomperidone is administered in three doses. In yet other aspects, deudomperidone is administered in four doses. In some embodiments, the deudomperidone is administered one time daily (QD). In other embodiments, the deudomperidone is administered two times daily (BID). In further embodiments, the deudomperidone is administered three times daily (TID). In yet other embodiments, the deudomperidone is administered four time daily (QID). In some aspects, a daily 10 mg dose of deudomperidone is administered 10 mg QD. In other aspects, a daily 10 mg dose of deudomperidone is administered 5 mg BID. In further aspects, a daily 20 mg dose of deudomperidone is administered 20 mg QD. In yet other aspects, a daily 20 mg dose of deudomperidone is administered 10 mg BID. In still further aspects, a daily 20 mg dose of deudomperidone is administered 5 mg QID. In other aspects, a daily 60 mg dose of deudomperidone is 30 mg BID. In further aspects, a daily 120 mg dose of deudomperidone is 60 mg BID. In yet other aspects, a daily 30 mg dose of deudomperidone is 10 mg TID. In still further aspects, a 30 mg daily dose of deudomperidone is 15 mg BID dose given as a combination of 10 mg of deudomperidone and 5 mg deudomperidone.

Prior to administration of a first dose of deudomperidone, the human has delayed gastric emptying. Gastric emptying may be measured by the gastric emptying breath test (GEBT). The GEBT is known in the art. Succinctly, the GEBT is a nonradioactive stable isotope breath test used in the art to diagnose or monitor gastroparesis. In this test, the ratio of exhaled ¹³CO₂/¹²CO₂ is used to determine the rate of gastric emptying after consumption of a standardized, ¹³C-enriched meal. In some embodiments, the ¹³C-enriched meal is ¹³C-Spirulina platensis. In some examples, the test includes consumption of a standardized, ¹³C-enriched meal after an 8-hour fast. See, e.g., the Cairn Gastric Emptying Breath Test (GEBT) Administration Guide at https://cairndiagnostics.com/wp-content/uploads/2019/05/Cairn-GEBT-How-To-Guide-updated-07May2019.pdf, which is hereby incorporated by reference. Breath samples are obtained one or more times prior to consumption of the ¹³C-enriched meal and then one or more times after meal consumption. The breath samples are then measured using, e.g., a gas isotope ratio mass spectrometer. Higher levels of ¹³C are indicative of delayed gastric emptying, i.e., that your stomach is not metabolizing the ¹³C-enriched meal. Lower levels of ¹³C are indicative of normal stomach emptying. In some embodiments, gastric emptying is measured at least about 10 days prior to administering the first dose of deudomperidone. In other embodiments, gastric emptying is measured 10 days prior to administering the first dose of deudomperidone. In further embodiments, gastric emptying is measured about 1 to about 10 days prior to administering the first dose of deudomperidone.

The human may also have a best-fit exponential emptying rate (T½) greater than about 80 minutes, as measured by GEBT using ¹³C-Spirulina platensis, prior to administering the deudomperidone. In some embodiments, the human has a T_{1/2} greater than about 100 minutes, as measured by GEBT using ¹³C-Spirulina platensis, prior to administering deudomperidone. In other embodiments, the human has a T_{1/2} greater than about 120 minutes, as measured by GEBT using ¹³C-Spirulina platensis, prior to administering deudomperidone. In further embodiments, the human has a T_{1/2} greater than about 140 minutes, as measured by GEBT using ¹³C-Spirulina platensis, prior to administering deudomperidone. In yet other embodiments, the human has a T_{1/2} greater than about 160 minutes, as measured by GEBT using ¹³C-Spirulina platensis, prior to administering deudomperidone. In still further embodiments, the human has a T₁₂ greater than about 180 minutes, as measured by GEBT using ¹³C-Spirulina platensis, prior to administering deudomperidone. In other embodiments, the human has a T_{1/2} greater than about 200 minutes, as measured by GEBT using ¹³C-Spirulina platensis, prior to administering deudomperidone.

Prior to administering the deudomperidone, the patient may have an American Neurogastroenterology and Motility Society Gastroparesis Cardinal Symptom Index Daily Diary (ANMS GCSI-DD) average composite score of about 2 or greater. As known in the art, the ANMS GCSI-DD covers 5 core relevant symptoms of gastroparesis: nausea, early satiety, postprandial fullness, upper abdominal pain and vomiting. Bloating is included as an exploratory symptom. Symptoms are rated on a severity numeric response scale from 0 (none) to 4 (very severe). Vomiting is captured on a frequency response scale and scored as follows: 0 for no episodes, 1 for one episode, 2 for 2 episodes, 3 for 3 episodes, and 4 for 4 or more episodes. In some embodiments, subjects recall symptoms over the previous 24 hours and completes the ANMS GCSI-DD patient-reported symptom questionnaire. In certain embodiments, the total ANMS GCSI-DD score is calculated as the mean of the 3 subscale scores for severity of nausea/vomiting, postprandial fullness/early satiety, and bloating. See, e.g., “The Use Manual for the ANMS GCSI-DD,” American Neurogastroenterology and Motility Society Gastroparesis Cardinal Symptom Index Daily Diary, 2018; https://www.fda.gov/media/125038/download. In other embodiments, the total ANMS GCSI-DD score is calculated using the ‘manual’ scoring method. The ANMS GCSI-DD manual scoring method is generated by summing the scores on each of the five symptom items (nausea, early satiety, postprandial fullness, upper abdominal pain, and number of vomiting episodes) and then dividing by 5, that is the number of items within the gastroparesis related symptom score. Thus, the maximum total symptom score could be (5 symptoms * maximum score 4 divided by 5); hence, the maximum score is 20/5=4. The ANMS GCSI-DD gastroparesis symptom daily score can range from 0 to 4. High scores on the ANMS GCSI-DD reflect greater symptom severity. See, e.g., “User Manuel for the ANMS GCSI-DD,” Federal Drug Administration at https://www.fda.gov/media/125038/download, which is hereby incorporated by reference. In some embodiments, the ANMS GCSI-DD average composite score is 2 prior to administering deudomperidone. In other embodiments, the ANMS GCSI-DD average composite score is 3 prior to administering deudomperidone. In further embodiments, the ANMS GCSI-DD average composite score is 4 prior to administering deudomperidone.

The patient further may have a ANMS GCSI-DD subscale score of 2 or greater, prior to administering deudomperidone. In some embodiments, the patient has an ANMS GCSI-DD nausea subscale score of 2 or greater, prior to administering deudomperidone. In other embodiments, the patient has an ANMS GCSI-DD nausea subscale score of 2 or greater for at least about 4 days, prior to administering deudomperidone. In other embodiments, the patient has an ANMS GCSI-DD early satiety/fullness subscale score of 2 or greater, prior to administering deudomperidone. In further embodiments, the patient has an ANMS GCSI-DD bloating subscale score of 2 or greater, prior to administering the deudomperidone.

Desirably, the methods result in increasing gastric emptying of the patient. In some embodiments, the increase in gastric emptying is measured by GEBT using ¹³C-Spirulina platensis, after administering deudomperidone. In some embodiments, the increase in gastric emptying is measured by GEBT using ¹³C-Spirulina platensis at least one day after administering deudomperidone. In further embodiments, the increase in gastric emptying is measured by GEBT at baseline and at the end of the study. In other embodiments, the increase in gastric emptying is measured by GEBT using ¹³C-Spirulina platensis about 2, about 3, about 4, about 5, about 6, about 7, about 8, about 9, about 10, about 11, about 12, about 13, about 14, about 15, or about 16 days after administering deudomperidone. In other embodiments, the increase in gastric emptying is measured by GEBT using ¹³C-spirulina platensis, about 2 to about 15, about 2 to about 14, about 2 to about 13, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 16, about 3 to about 15, about 3 to about 14, about 3 to about 13, about 3 to about 12, about 3 to about 11, about 3 to about 10, about 3 to about 9, about 3 to about 8, about 3 to about 7, about 3 to about 6, about 3 to about 5, about 3 to about 4, about 4 to about 16, about 4 to about 15, about 4 to about 14, about 4 to about 13, about 4 to about 12, about 4 to about 11, about 4 to about 10, about 4 to about 9, about 4 to about 8, about 4 to about 7, about 4 to about 6, about 4 to about 5, about 5 to about 16, about 5 to about 15, about 5 to about 14, about 5 to about 13, about 5 to about 12, about 5 to about 11, about 5 to about 10, about 5 to about 9, about 5 to about 8, about 5 to about 7, about 5 to about 6, about 6 to about 16, about 6 to about 15, about 6 to about 12, about 6 to about 11, about 6 to about 10, about 6 to about 9, about 6 to about 8, about 6 to about 7, about 7 to about 16, about 7 to about 15, about 7 to about 14, about 7 to about 13, about 7 to about 12, about 7 to about 11, about 7 to about 10, about 7 to about 9, about 7 to about 8, about 8 to about 16, about 8 to about 15, about 8 to about 14, about 8 to about 13, about 8 to about 12, about 8 to about 11, about 8 to about 10, about 8 to about 9, about 9 to about 16, about 9 to about 15, about 9 to about 14, about 9 to about 13, about 9 to about 12, about 9 to about 11, about 9 to about 10, about 10 to about 16, about 10 to about 16, about 10 to about 14, about 10 to about 13, about 10 to about 12, about 10 to about 11, about 11 to about 16, about 11 to about 15, about 11 to about 14, about 11 to about 13, about 11 to about 12, about 12 to about 16, about 12 to about 15, about 12 to about 14, about 12 to about 13, about 13 to about 16, about 13 to about 15, about 13 to about 14, about 14 to about 16, about 14 to about 15, or about 15 to about 16 days after administering deudomperidone. In further embodiments, the increase in gastric emptying is measured by GEBT using ¹³C-Spirulina platensis, about 12 to about 14 days after administering the deudomperidone.

Advantageously, administration of deudomperidone results in a clinically significant improvement in nausea (such as postprandial nausea), early satiety, postprandial fullness, upper abdominal pain, vomiting, bloating, or a combination thereof, as measured by an ANMS GCSI-DD score after treatment with deudomperidone, such as about 6 to about 12 weeks after administration. In certain aspects, administration of deudomperidone results in a clinically significant improvement in postprandial nausea, early satiety, postprandial fullness, upper abdominal pain, vomiting, and bloating as measured by an ANMS GSI-DD score about 6, about 7, about 8, about 9, about 10, about 11, or about 12 weeks after administration. In other aspects, administration of deudomperidone results in a clinically significant improvement in postprandial nausea, early satiety, postprandial fullness, upper abdominal pain, vomiting, and bloating as measured by an ANMS GSI-DD score about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks after administration. In some embodiments, administration of deudomperidone results in a clinically significant improvement in nausea, as measured by an ANMS GCSI-DD score after treatment with deudomperidone. In other embodiments, administration of deudomperidone results in a clinically significant improvement in early satiety, as measured by an ANMS GCSI-DD score after treatment with deudomperidone. In further embodiments, administration of deudomperidone results in a clinically significant improvement in postprandial fullness, as measured by an ANMS GCSI-DD score after treatment with deudomperidone. In yet other embodiments, administration of deudomperidone results in a clinically significant improvement in upper abdominal pain, as measured by an ANMS GCSI-DD score after treatment with deudomperidone. In still further embodiments, administration of deudomperidone results in a clinically significant improvement in vomiting, as measured by an ANMS GCSI-DD score after treatment with deudomperidone. In other embodiments, administration of deudomperidone results in a clinically significant improvement in bloating, as measured by an ANMS GCSI-DD score after treatment with deudomperidone.

Administration of deudomperidone also results in a clinically significant improvement in an average composite score of the ANMS GCSI-DD score after the treatment, such as about 6 to about 12 weeks after administration. In certain aspects, administration of deudomperidone may result in a clinically significant improvement in an average composite score of the ANMS GCSI-DD score after about 6, about 7, about 8, about 9, about 10, about 11, or about 12 weeks of administration. In other aspects, administration of deudomperidone may result in a clinically significant improvement in an average composite score of the ANMS GCSI-DD score after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration. In some embodiments, the ANMS GCSI-DD average composite score decreases by 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 points after administration of deudomperidone. In other embodiments, the ANMS GCSI-DD average composite score decrease by one point after administration of deudomperidone. In other embodiments, the ANMS GCSI-DD average composite score decreases by two points after administration of deudomperidone. In further embodiments, the ANMS GCSI-DD average composite score decreases by three points after administration of deudomperidone. In yet other embodiments, the ANMS GCSI-DD average composite score decreases by four points after administration of deudomperidone.

Administration of deudomperidone further results in a clinically significant improvement in an ANMS GCSI-DD nausea subscale score after the treatment, such as about 6 to about 12 weeks after administration. In certain aspects, administration of deudomperidone may result in a clinically significant improvement in a nausea subscale score of the ANMS GCSI-DD score after about 6, about 7, about 8, about 9, about 10, about 11, or about 12 weeks of administration. In other aspects, administration of deudomperidone may result in a clinically significant improvement in a nausea subscale score of the ANMS GCSI-DD score after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration. In some embodiments, the ANMS GCSI-DD nausea subscale score decreases by 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 points. In further embodiments, the ANMS GCSI-DD nausea subscale decreases by one point after administration of deudomperidone. In other embodiments, the ANMS GCSI-DD nausea subscale score decreases by two points after administration of deudomperidone. In further embodiments, the ANMS GCSI-DD nausea subscale score decreases by three points after administration of deudomperidone. In yet other embodiments, the average composite score decreases by four points after administration of deudomperidone.

Administration of deudomperidone further results in a clinically significant improvement in gastric emptying as measured by GEBT after about 12 to about 16 days after treatment with deudomperidone. In some embodiments, administration of deudomperidone further results in a clinically significant improvement in gastric emptying as measured by GEBT after about 12, about 13, about 14, about 15, or about 16 days after treatment with deudomperidone. In other embodiments, administration of deudomperidone further results in a clinically significant improvement in gastric emptying as measured by GEBT after about 12 to about 16, about 12 to about 15, about 12 to about 14, about 12 to about 13, about 13 to about 16, about 13 to about 15, about 13 to about 14, about 14 to about 16, about 14 to about 15, or about 15 to 16 days after treatment with deudomperidone. In further embodiments, administration of deudomperidone further results in a clinically significant improvement in gastric emptying as measured by GEBT after about 12 days after treatment with deudomperidone. In yet other embodiments, administration of deudomperidone further results in a clinically significant improvement in gastric emptying as measured by GEBT after about 13 days after treatment with deudomperidone. In still further embodiments, administration of deudomperidone further results in a clinically significant improvement in gastric emptying as measured by GEBT after about 14 days after treatment with deudomperidone. In other embodiments, administration of deudomperidone further results in a clinically significant improvement in gastric emptying as measured by GEBT after about 15 days after treatment with deudomperidone. In further embodiments, administration of deudomperidone further results in a clinically significant improvement in gastric emptying as measured by GEBT after about 16 days after treatment with deudomperidone.

Administration of deudomperidone also results in a clinically significant improvement in gastric emptying as measured by GEBT and in symptoms measured by ANMS GCSI-DD total scores after the treatment.

Administration of deudomperidone further results in a clinically significant improvement in gastric emptying as measured by GEBT after the treatment.

In yet other embodiments, administration of deudomperidone may result in a clinically significant improvement in a vomiting subscale score of the ANMS GCSI-DD score after administration, such as about 6 to about 12 weeks after administration. In certain aspects, administration of deudomperidone may result in a clinically significant improvement in a vomiting subscale score of the ANMS GCSI-DD score after about 6, about 7, about 8, about 9, about 10, about 11, or about 12 weeks of administration. In other aspects, administration of deudomperidone may result in a clinically significant improvement in a vomiting subscale score of the ANMS GCSI-DD score after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration.

In still further embodiments, administration of deudomperidone may result in a clinically significant improvement in a total score of the ANMS CSI-DD score after administration, such as about 6 to about 12 weeks after administration. In certain aspects, administration of deudomperidone may result in a clinically significant improvement in a total score of the ANMS GCSI-DD score after about 6, about 7, about 8, about 9, about 10, about 11, or about 12 weeks of administration. In other aspects, administration of deudomperidone may result in a clinically significant improvement in a total score of the ANIS GCSI-DD score after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration.

In other embodiments, administration of deudomperidone may result in a clinically significant improvement in an early satiety subscale score of the ANMS GCSI-DD score after administration, such as about 6 to about 12 weeks after administration. In certain aspects, administration of deudomperidone may result in a clinically significant improvement in an early satiety subscale score of the ANMS GCSI-DD score after about 6, about 7, about 8, about 9, about 10, about 11, or about 12 weeks of administration. In other aspects, administration of deudomperidone may result in a clinically significant improvement in an early satiety subscale score of the ANMS GCSI-DD score after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration.

In further embodiments, administration of deudomperidone may result in a clinically significant improvement in a post-prandial fullness subscale score of the AN NIS GCSI-DD score after administration, such as about 6 to about 12 weeks after administration. In certain aspects, administration of deudomperidone may result in a clinically significant improvement in a post-prandial fullness subscale score of the ANMS GCSI-DD score after about 6, about 7, about 8, about 9, about 10, about 11, or about 12 weeks of administration. In other aspects, administration of deudomperidone may result in a clinically significant improvement in a post-prandial fullness subscale score of the ANIS GCSI-DD score after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11 about 8 to about 10, about 8 to about 9, about 9 to about 12, about 9 to about 11, about 9 to about 10, about 10 to about 12, about 10 to about 11, or about 11 to about 12 weeks of administration.

In still other embodiments, administration of deudomperidone may result in a clinically significant improvement in an upper abdominal pain subscale score of the ANMS GCSI-DD score after administration, such as about 6 to about 12 weeks after administration. In certain aspects, administration of deudomperidone may result in a clinically significant improvement in an upper abdominal pain subscale score of the ANMS GCSI-DD score after about 6, about 7, about 8, about 9, about 10, about 11, or about 12 weeks of administration. In other aspects, administration of deudomperidone may result in a clinically significant improvement in an upper abdominal pain subscale score of the ANMS GCSI-DD score after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration.

In other embodiments, administration of deudomperidone may result in a clinically significant improvement in gastric emptying as measured by ANMS GCSI-DD subscale scores after administration, such as about 6 to about 12 weeks after administration. In certain aspects, administration of deudomperidone may result in a clinically significant improvement in gastric emptying as measured by ANMS GCSI-DD subscale scores after about 6, about 7, about 8, about 9, about 10, about 11, or about 12 weeks of administration. In other aspects, administration of deudomperidone may result in a clinically significant improvement in gastric emptying as measured by ANMS GCSI-DD subscale scores after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration.

In yet other embodiments, administration of deudomperidone may result in a clinically significant improvement in gastric emptying as measured by ANMS GCSI-DD total scores after administration, such as about 6 to about 12 weeks after administration. In certain aspects, administration of deudonperidone may result in a clinically significant improvement in gastric emptying as measured by ANMS GCSI-DD total scores after about 6, about 7, about 8, about 9, about 10, about 11, or about 12 weeks of administration. In other aspects, administration of deudomperidone may result in a clinically significant improvement in gastric emptying as measured by ANMS GCSI-DD total scores after about 6 to about 11, about 6 to about 10, about 6 to about 9, about 6 to about 8, about 6 to about 7, about 7 to about 12, about 7 to about 11, about 7 to about 10, about 7 to about 9, about 7 to about 8, about 8 to about 12, about 8 to about 11, about 8 to about 10, about 8 to about 9, about 9 to about 12, about 9 to about 11, about 9 to about 10, about 10 to about 12, about 10 to about 11, or about 11 to about 12 weeks of administration.

The deudomperidone formulations described herein are useful in a variety of treatment methods including, without limitation, methods for treating a disorder that is gastroparesis, nausea apart from gastroparesis, vomiting apart from gastroparesis, nausea associated with gastroparesis, vomiting associated with gastroparesis, gastroesophageal reflux disease, insufficient lactation, nausea and/or vomiting associated with chemotherapy, or a combination thereof. The methods include administering to the patient a pharmaceutical formulation described herein. In some embodiments, the methods are useful for treating gastroparesis. In other embodiments, the methods are useful for treating nausea apart from gastroparesis. In further embodiments, the methods are useful for treating vomiting apart from gastroparesis. In yet other embodiments, the methods are useful for treating nausea associated with gastroparesis. In still further embodiments, the methods are useful for treating vomiting associated with gastroparesis. In other embodiments the methods are useful for treating gastroesophageal reflux disease. In further embodiments, the methods are useful for treating insufficient lactation. In still other embodiments, the methods are useful for treating nausea and/or vomiting associated with chemotherapy.

The pharmaceutical formulations may be administered by any route that would be acceptable for humans. In some embodiments, the pharmaceutical formulations the administration is oral, transdermal, parenteral, or a combination thereof. In further embodiments, administration is oral.

The pharmaceutical formulations may be formulated for administration in solid or liquid forms. In some embodiments, the pharmaceutical formulations are formulated in the form of a tablet, caplet, capsule, powder, softgel, suspension or liquid, or a combination thereof. In other embodiments, the pharmaceutical formulations are formulated in the form of a tablet. In further embodiments, the pharmaceutical formulations are formulated in the form of a caplet. In yet other embodiments, the pharmaceutical formulations are formulated in the form of a capsule. In still further embodiments, the pharmaceutical formulations are formulated in the form of a powder. In other embodiments, the pharmaceutical formulations are formulated in the form of a softgel. In further embodiments, the pharmaceutical formulations are formulated in the form of suspension. In yet other embodiments, the pharmaceutical formulations are formulated in the form of a liquid.

Administering deudomperidone according to the disclosure does not cause or result in a clinically significant QT prolongation. For example, a patient's ΔΔQTcF after deudomperidone administration according to the disclosure is 10 ms or less than about 10 ms (e.g., 10, 9, 8, 7, 6, 5, 4, 3, 2, 1, or 0 ms). In some aspects, a patient's ΔΔQTcF after deudomperidone administration according to the disclosure is about 1.5 ms or less, for example, 1.5, 1.4, 1.3, 1.2, 1.1, 1, 0.9, 0.8, 0.7, 0.6, 0.5, 0.4, 0.3, 0.2, 0.1, or 0 ms. In some aspects, a patient's ΔΔQTcF after administration of about 30 mg or about 20 mg or about 10 mg of deudomperidone according to the disclosure is about 1.5 ms or less, for example, 1.5, 1.4, 1.3, 1.2, 1.1, 1, 0.9, 0.8, 0.7, 0.6, 0.5, 0.4, 0.3, 0.2, 0.1, or 0 ms. In some aspects, a patient's ΔΔQTcF after administration of about 100 mg or about 90 mg or about 80 mg or about 70 mg or about 60 mg or about 50 mg or about 40 mg of deudomperidone according to the disclosure is about 3.5 ms or less, for example, 3.5, 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, or 0 ms.

Additionally, administering deudomperidone according to the disclosure does not cause or result in a clinically significant change in the patient's heart rate. In further aspects, administering deudomperidone according to the disclosure does not cause or result in a clinically significant change in the cardiac conduction of the patient. In other aspects, the pulse rate (PR) interval of the patient is about 0.12 to about 0.2 seconds (e.g., 0.12, 0.13, 0.14, 0.15, 0.16, 0.17, 0.18, 0.19, 0.2 seconds), after administration of deudomperidone according to the disclosed methods. In some aspects, the QRS interval of the patient is about 0.08 and 0.10 seconds (e.g., 0.08, 0.09, 0.10 seconds) after administration of the deudomperidone.

The following Example is provided to illustrate some of the concepts described within this disclosure. While the Example is considered to provide specific individual embodiments of formulations, methods of preparation and use, it should be considered to limit the more general embodiments described herein.

In the following Example, efforts have been made to ensure accuracy with respect to numbers used (e.g., amounts, temperature, etc.) but some experimental error and deviation should be accounted for.

| Abbreviation | Definition |
|---|---|
| λz | Apparent first-order terminal elimination rate constant |
| ¹³C | Carbon-13 |
| AE | Adverse event |
| ANMS | American Neurogastroenterology and Motility Society |
| GCSI-DD | Gastroparesis Cardinal Symptom Index Daily Diary |
| ALT | Alanine aminotransferase |
| AST | Aspartate aminotransferase |
| AUC | Area under the plasma concentration-time curve |
| AUC_{% extrap} | Percent of the area under the plasma concentration-time |
| | curve from pre-dose (time 0) to infinity extrapolated |
| AUC_{0-∞} | Area under the plasma concentration-time curve from |
| | pre-dose (time 0) to infinity |
| AUC₀₋₁₂ | Area under the plasma concentration-time curve from |
| | pre-dose (time 0) to 12 hours |
| AUC₀₋ₜ | Area under the plasma concentration-time curve from |
| | pre-dose (time 0) to the last quantifiable plasma |
| | concentration |
| AUCₜₐᵤ | Area under the plasma concentration-time curve over |
| | the dosing interval |
| AV | Atrioventricular |
| BID | Twice daily |
| BMI | Body mass index |
| BT_{1/2} | Gastric Emptying Breath Test T_{1/2} (BT T_{1/2}) |
| Cₘₐₓ | Maximum plasma concentration |
| CKD-EPI | Chronic Kidney Disease Epidemiology Collaboration |
| CI | Confidence interval |
| Cₗₐₛₜ | Last quantifiable plasma concentration |
| CL/F | Apparent total plasma clearance after oral administration |
| CNS | Central nervous system |
| COVID-19 | Coronavirus Disease 2019 |
| CPGAS | Clinical Patient Grading Assessment Scale |
| CV | Coefficient of variation |
| CYP | Cytochrome P450 |
| DGE | Delayed gastric emptying |
| DRC | Data Review Committee |
| ECG | Electrocardiogram |
| eGFR | Estimated glomerular filtration rate |
| FD&C | Food, drug, and cosmetic |
| FDA | United States Food and Drug Administration |
| FSH | Follicle-stimulating hormone |
| GEBT | Gastric emptying breath test |
| GEBT T½ | Gastric emptying breath test half-life |
| GI | Gastrointestinal |
| GLP-1 | Glucagon-like peptide-1 receptor |
| GM | Geometric mean |
| HbsAg | Hepatitis B surface antigen |
| HCV | Hepatitis C virus |
| HIV | Human immunodeficiency virus |
| I/E | Inclusion and exclusion |
| ITT | Intent-to-Treat |
| IV | Intravenous |
| LB | Lime bone |
| LLOQ | Lower limit of quantification |
| MAD | Multiple ascending dose |
| MedDRA | Medical Dictionary for Regulatory Activities |
| MITT | Modified Intent-to-Treat |
| MSEC | Millisecond(s) |
| NF | United States National Formulary |
| PAGI-SYM | Patient Assessment of Gastrointestinal Disorders |
| | Symptom Severity Index |
| PD | Pharmacodynamic(s) |
| PEG | Percutaneous endoscopic gastrostomy |
| PGx | Pharmacogenomic |
| PK | Pharmacokinetic(s) |
| POC | Point-of-care |
| PP | Per-Protocol |
| PRO | Patient-reported outcomes |
| PT | Preferred term |
| QD | Once daily |
| QTc | Heart rate-corrected QT interval |
| QTcF | Heart rate-corrected QT interval using Fridericia's formula |
| R_{AUC} | Accumulation ratio based on area under the plasma |
| | concentration-time curve over the dosing interval after |
| | the first dose and the final dose |
| R_{cmax} | Accumulation ratio based on maximum plasma concen- |
| | tration after the first dose and the final dose |
| RNA | Ribonucleic acid |
| SAD | Single ascending dose |
| SAE | Serious adverse event |
| SD | Standard deviation |
| SGLT2 | Sodium-glucose cotransporter-2 |
| SOC | System organ class |
| T_{1/2} | Terminal phase elimination half-life |
| TEAE | Treatment-emergent adverse event |
| TID | 3 times daily |
| Tₘₐₓ | Time to maximum plasma concentration |
| TSH | Thyroid-stimulating hormone |
| ULN | Upper limit of normal |
| USP | United States Pharmacopoeia |
| Vz/F | Apparent volume of distribution during terminal |
| | elimination phase after oral administration |

### Example 1

This is a randomized, double-blind, placebo-controlled Phase 2A study to evaluate safety, efficacy, PK, and dose response of oral deudomperidone in adults with idiopathic or diabetic gastroparesis.

The study will begin by randomizing subjects in Cohort 1 to either deudomperidone 10 mg BID or placebo BID in a ratio such that 15 subjects receive deudomperidone and 5 subjects receive placebo. Subjects will opt into either a sparse pharmacokinetic sampling subgroup or an intensive PK sampling subgroup. The study will be stratified at randomization by idiopathic versus diabetic gastroparesis. Dose levels for Cohorts 2 and 3 are 20 mg QD and 5 mg BID, respectively, with 15 subjects receiving the study drug and 5 subjects receiving placebo in each cohort. The highest dose will not exceed 60 mg BID.

### Subjects Will be Evaluated as Follows:

Days −28 to −11: Screening procedures will be performed. The ANMS GCSI-DD questionnaire will be completed daily for at least 14 days prior to randomization.
Day −10 to −3: The ANMS GCSI-DD questionnaire will be completed daily. A GEBT will be performed on a single day within this window with breath samples obtained twice prior to consumption of a standardized, ¹³C-enriched meal and then at 45, 90, 120, 150, 180, and 240 minutes after meal consumption after an 8-hour fast. Prior to the start of the GEBT, the baseline PAGI-SYM will be completed.
Day −3 to −1: A Pre-Randomization visit will be completed to perform safety assessments and to allow adequate turnaround time of lab results prior to Day 1 (Randomization).
Days 1 to 14: Study drug will be administered (deudomperidone or placebo BID for 14 (±2 days) and safety assessments will be performed; ANMS GCSI-DD questionnaire will be completed daily and PK blood sampling will be completed at specified timepoints. On Day 14 (±2 days) the PAGI-SYM and Clinical Grading Assessment questionnaires will be completed prior to the final dose of study drug.
Day 14 (±2 days): A GEBT will be administered 30 minutes after study drug administration on Day 14 (±2 days). Breath samples will be obtained twice prior to ¹³C enriched meal consumption and then at 45, 90, 120, 150, 180, and 240 minutes after meal consumption after an 8-hour fast.

Subjects will be stratified at randomization based on their etiology for gastroparesis (i.e., diabetic gastroparesis versus idiopathic gastroparesis).

On days of the GEBT, subjects will present at the clinic after an overnight fast and should take regular medications, including any treatment for diabetes, with the exception of any prohibited concomitant medication. Subjects will self-administer their usual morning insulin injection, taking into consideration their fasting status. Subjects will be instructed to bring in their study medication and additional insulin, as needed, to the clinic. Fasting blood glucose will be assessed before study drug administration and gastric emptying assessments to ensure a blood glucose of ≤275 mg/dL. Additional insulin may be given (prorated based on meal caloric content) to ensure a blood glucose of ≤275 mg/dL prior to the gastric emptying procedure.

Subjects will have follow-up visit(s) 1 to 3 days after the final dose of study drug to assess adverse events, changes to concomitant medications (including use of rescue medications), vital signs, and for PK sampling. Subjects participating in the intensive PK subgroup will be seen in the clinic on Day 3 (±1 day), 7 (±1 day), 10 (±1 day), 14 (±2 day), Day 15 (±1 day), and Day 16 (±1 day). Subjects participating in the sparse PK subgroup will have the option to complete either the Day 15 Visit or the Day 16 (±1 day) Visit.

### A. Study Objectives

### (i) Objectives

The objectives of this study are the following:
To evaluate the safety and tolerability of multiple doses of deudomperidone in subjects with idiopathic or diabetic gastroparesis
To evaluate the effect of multiple doses of deudomperidone on gastric emptying as measured by the GEBT in subjects with idiopathic or diabetic gastroparesis
To characterize the PK of multiple doses of deudomperidone in subjects with idiopathic or diabetic gastroparesis

### (ii) Exploratory Objectives

The exploratory objectives of this study include the following:
To assess the effect of multiple doses of deudomperidone on nausea associated with gastroparesis based on the nausea score on the ANMS GCSI-DD
To assess the effect of multiple doses of deudomperidone on ANMS GCSI-DD scores (total and subscales) in subjects with idiopathic or diabetic gastroparesis
To assess the effect of multiple doses of deudomperidone on a variety of patient reported outcomes
To characterize exposure-response relationships of deudomperidone

### B. Inclusion Criteria

Patients who meet all of the following criteria will be eligible to participate in the study:
1. Male and female patients 18 to 70 years old.
2. Has a current diagnosis of idiopathic or diabetic gastroparesis as defined by the following:
   Has GI symptoms felt to be consistent with gastroparesis (e.g., postprandial nausea/vomiting, postprandial fullness, early satiety, bloating, and/or epigastric/abdominal pain) within the 6 months prior to screening; or
   Has documented DGE within the past 3 years as determined by GEBT or scintigraphy. Patients who have DGE documented through other modalities (e.g., SmartPill™, manometry, etc.) or who have had an abnormal GEBT or scintigraphy >3 years ago may be considered.
3. Has a BMI between 18 and 40 kg/m², inclusive.
4. Has glycosylated hemoglobin level ≤11% at Screening.
5. Male patients with female partners of child-bearing potential must agree to use 2 medically accepted, highly effective methods of birth control from Day 1 through 60 days following the final dose of study drug. Medically accepted, highly effective methods of birth control for male patients with female partners of child-bearing potential include the following: latex condom with spermicide, diaphragm with intravaginal spermicide, cervical cap with spermicide, indwelling intrauterine device (hormonal or nonhormonal), implanted contraceptives, and oral contraceptives.
6. Male patients must agree to abstain from sperm donation from Day 1 through 60 days following the final dose of study drug.
7. Female patients with male partners must be surgically sterile (hysterectomy and/or bilateral oophorectomy), postmenopausal for at least 1 year (with confirmed FSH in postmenopausal range at the Screening Visit), or agree to use 2 medically accepted, highly effective methods of birth control from Day −14 until 60 days following the final dose of study drug. Medically accepted, highly effective methods of birth control for female patients with male partners include the following: latex condom with spermicide, diaphragm with intravaginal spermicide, cervical cap with spermicide, and nonhormonal indwelling intrauterine device.
8. Has a negative breath alcohol and urine drug screen (including tetrahydrocannabinol) at the Screening Visit and at the time of randomization.
9. Is willing to abstain from grapefruit, grapefruit products, star fruit, star fruit products, and Seville oranges from 48 hours prior to randomization until end of study.
10. Willing to refrain from long-acting GLP-1 agonists, SGLT-2 inhibitors, or pramlintide.

### C. Exclusion Criteria

Patients who meet any of the following criteria will be excluded from participation in the study:
1. Has a history of, or current, clinically significant arrhythmias, including ventricular tachycardia, ventricular fibrillation, and Torsades de pointes. Patients with minor forms of ectopy (e.g., premature atrial contractions) are not necessarily excluded.
2. Has clinically significant bradycardia with a resting heart rate under 50 beats per minute, sinus node dysfunction, or heart block.
3. Has prolonged heart rate-corrected QTcF (QTcF >450 msec for males or QTcF >470 msec for females) based on the average of triplicate ECGs.
4. Has a personal or family history of long QT syndrome, Torsades de pointes, or other complex ventricular arrhythmias or family history of sudden death.
5. Has evidence (based on screening or baseline assessments) or history of clinically significant immunologic, hematologic, renal, endocrine, pulmonary, GI, cardiovascular, hepatic, psychiatric, neurologic, or allergic disease (including drug allergies); surgical conditions; cancer (with the exception of basal or squamous cell carcinoma of the skin and cancer that resolved or has been in remission for >5 years prior to the Screening Visit); or any condition that might significantly interfere with the absorption, distribution, metabolism, or excretion of the study drug.
6. Has a history of prolactin-releasing pituitary tumor (i.e., prolactinoma).
7. Has elevated serum prolactin.
8. Has known or suspected hypogonadism, current clinically significant menstrual abnormalities (e.g., oligomenorrhea or amenorrhea), gynecomastia, galactorrhea, or other clinical features that may be consistent with hyperprolactinemia.
9. Has pyloric injection of botulinum toxin within 6 months of screening and/or during the course of the study.
10. Has bilirubin, alkaline phosphatase, AST, or ALT levels greater than 2× the upper limit of normal or has a Child-Pugh classification grade of B or C.
11. Has a serum creatinine level greater than 1.5× the upper limit of normal or has an eGFR <30 mL/min/1.73 m² using the CKD-EPI equation at screening.
12. Has a hemoglobin level <10 g/dL at screening.
13. Has TSH levels that are abnormal at screening.
14. Has inability to perform a breath test.
15. Has known allergy to eggs or spirulina.
16. Use of investigational, prescription, or over-the-counter medications as follows:
   Actively participating in an experimental therapy study; received experimental therapy with a small molecule within 30 days of randomization or 5 half-lives (whichever is longer); or received experimental therapy with a large molecule within 90 days of randomization or 5 half-lives, (whichever is longer).
   Use of a concomitant CYP3A4 inhibitor (whether a medication, herbal supplement, dietary supplement, or nutraceutical) within 14 days or 5 half-lives (whichever is longer) of the first dose of study drug until the end of the study.
   Use of a concomitant CYP3A4 inducer (whether a medication, herbal supplement, dietary supplement, or, nutraceutical) within 28 days or 5 half-lives (whichever is longer) of the first dose of study drug or inducer until the end of the study.
   Use of any medication, herbal supplements, dietary supplements, or nutraceuticals with potential to prolong QT within 28 days prior to the first dose of study drug until the end of the study.
   Use of motility agents (including but not limited to metoclopramide, domperidone, erythromycin, etc.), other medications that may affect gastric emptying (e.g., opiates, marijuana, and anticholinergics), and antiemetics (except for protocol-specified rescue antiemetic medication) within 14 days prior to baseline GEBT until the end of the study. Gastric pacemakers should be turned off within 14 days prior to baseline GEBT and kept off until end of study.
17. History of alcoholism or drug abuse within 2 years prior to dosing as defined by the Diagnostic and Statistical Manual of Mental Disorders, 4^{th} Edition.
18. Typical consumption of ≥14 alcoholic drinks weekly.
19. Evidence of ongoing illicit drug use.
20. Positive for HIV antibody, HCV by RNA, or HbsAg at the Screening Visit.
21. Donated blood or blood products within 30 days prior to dosing.
22. Currently undergoing treatment with weight loss medication or prior weight loss surgery (e.g., gastric bypass surgery).
23. Pregnant, breastfeeding, or planning to become pregnant during the course of study.
24. Inability to swallow medication.
25. Currently receiving parenteral feeding or presence of a nasogastric or other enteral tube (e.g., PEG tube) for feeding or decompression.
26. Known or suspected gastric outlet obstruction (e.g., peptic stricture) or other GI mechanical obstruction as documented by upper GI tract endoscopy or upper GI radiographic series in the past 3 years.
27. Known history or current diagnosis of intestinal malabsorption or pancreatic exocrine disease. 28. History of gastric surgery such as fundoplication, gastrectomy, vagotomy, pyloroplasty, or bariatric procedure.
29. History or presence of any medical condition or psychiatric disease, which could interfere with the conduct of the study or would put the patient at unacceptable risk.
30. Prior lack of response to domperidone or known hypersensitivity or intolerance to domperidone or any of the excipients in the deudomperidone formulation.

Subjects must also meet the following criteria at the time of randomization:
Has been off all motility agents and antiemetics for at least 14 days and is willing to remain off such medications (except for protocol-specified antiemetic rescue medication) during the course of the study.
Demonstrates a confirmed diagnosis of idiopathic or diabetic gastroparesis as defined by the following:
   Current upper GI symptoms (e.g., nausea/vomiting, postprandial fullness, early satiety, bloating, and/or epigastric/abdominal pain).
   Documented DGE within the 10 days prior to first dose of study drug based on GEBT using ¹³C-Spirulina platensis demonstrating T_{1/2}>80 minutes. Subjects with a T_{1/2}>200 minutes may be considered.
Has an ANMS GCSI-DD score that satisfies the following:
   An average composite score of 2 (utilizing a Likert scale from 0 [none] to 4 [very severe]) for the last 10 days prior to randomization. The predominant symptom experienced should not be abdominal pain.
   A nausea subscale score (comprising nausea, retching, and vomiting) 2 at least 4 of 7 days OR at least 1 episode of vomiting during the portion of the ScreeningPeriod during which the subject has not been taking motility agents or antiemetics (with the exception of protocol-specified rescue medication).

### D. Study Treatment

### (i) Treatment Groups

This study is planned to include 4 treatment groups with 15 subjects each.

At randomization, subjects who meet all eligibility criteria (based on inclusion/exclusion and randomization criteria) will be randomized to either deudomperidone 10 mg BID or placebo BID in a 3:1 ratio. The study will begin by randomizing subjects in Cohort 1 to either deudomperidone 10 mg BID or placebo BID such that 15 subjects receive deudomperidone and 5 subjects receive placebo. Currently planned dose levels for Cohorts 2 and 3 are 20 mg QD and 5 mg BID, respectively, with 15 subjects receiving study drug and 5 subjects receiving placebo in each cohort.

The duration of the double-blind treatment period will be 14 days (+2 days) of study drug.

### (ii) Drug Supplies

Deudomperidone capsules consist of deudomperidone drug product in a size 2C, oval-shaped, blue, softgel capsule. Each deudomperidone capsule will contain either 5 or 10 mg of active deudomperidone drug substance. The deudomperidone capsules will also contain inactive ingredients.

A matching placebo softgel capsule containing the same inactive ingredients without deudomperidone drug substance will also be provided.

(iii) Study Drug Administration

All subjects will take a dose of blinded study drug (deudomperidone or placebo capsules) orally at 8:00 AM (±2 hours) and 8:00 PM (±2 hours) with approximately 240 mL of water. A 12-hour interval will be maintained between each dose for a given individual. All subjects should make an effort to fast for a minimum of 8 hours before the morning dose on Day 1. However, a light meal may be consumed ≥2 hours prior to the morning dose of study drug if food should be consumed by the subject (i.e., due to low blood glucose levels or symptoms of hypoglycemia). Subjects providing sparse PK samples should continue to fast for a minimum of 2 hours and subjects providing more intensive PK samples should continue to fast for a minim of 4 hours after the morning dose on Day 1. However, if food should be consumed, it is permissible to break the specified fast to ensure subject safety. On Day 14 [±2 days], all subjects will fast for a minimum of 8 hours before the morning dose of study drug and 4 hours following the completion of the GEBT meal. Subjects will be required to fast 2 hours before and 1 hour after all other doses.

E. Excluded Medications, Foods, and/or Procedures

Use of the following investigational, prescription, or over-the-counter medications is not permitted during the study:
Pramlintide
SGLT2 inhibitors or GLP-1 agonists with long half-lives [e.g., Bydureon® (exenatide extended release), Victoza® (liraglutide), Tanzeum™ (albiglutide), or Trulicity® (dulaglutide)].
Experimental therapy with a small molecule within 30 days prior to randomization or 5 half-lives (whichever is longer); or experimental therapy with a large molecule within 90 days prior to randomization or 5 half-lives (whichever is longer).
CYP3A4 inhibitors (whether a medication, herbal supplement, dietary supplement, nutraceutical, or food) within 14 days or 5 half-lives (whichever is longer) of the first dose of study drug or inducer until the end of the study.
CYP3A4 inducers (whether a medication, herbal supplement, dietary supplement, nutraceutical, or food) within 28 days or 5 half-lives (whichever is longer) of the first dose of study drug until the end of the study.
Medication, herbal supplements, dietary supplements, or nutraceuticals with potential to prolong QT within 28 days prior to the first dose of study drug until the end of the study.
Motility agents (including but not limited to metoclopramide, domperidone, erythromycin, etc.), other medications that may affect gastric emptying (e.g., opiates, marijuana, and anticholinergics), and antiemetics (except for protocol-specified rescue antiemetic medication) within 14 days prior to baseline GEBT until the end of the study. Gastric pacemakers should be turned off within 14 days prior to baseline GEBT and kept off until end of study.
Pyloric injection of botulinum toxin within 6 months of screening and during the course of the study.
Weight loss medication or prior weight loss surgery (e.g., gastric bypass surgery).
Parenteral feeding or presence of a nasogastric or other enteral tube (e.g., PEG tube) for feeding or decompression.

### F. Procedures

Baseline GEBT will be performed between Days −10 and −3 after a minimum 14-day washout from motility agents.

The following procedures will be performed at the Baseline GEBT misit (Days −10 to −3):
Assess fasting blood glucose before gastric emptying assessments to ensure a blood glucose of 275 mg/dL.
PAGI-SYM questionnaire will be completed at the site prior to initiation of the GEBT.
Obtain breath samples for GEBT twice prior to consumption of a standardized, ¹³C-enriched meal per the study manual and then at 45, 90, 120, 150, 180, and 240 minutes after meal consumption after an 8-hour fast.
Record prior medications.
Obtain vital signs (including heart rate, blood pressure, respiration rate, and temperature) while subject is sitting after a minimum 5-minute rest.
ANMS GCSI-DD questionnaire will be completed every evening.

### G. Efficacy Assessments

### (i) Primary Efficacy Assessment

The primary efficacy variable is the change from baseline (gathered on Days −10 to −3) in gastric emptying as measured by GEBT at Day 14 (±2 days).

The GEBT is a nonradioactive stable isotope breath test, in which the ratio of exhaled ¹³CO₂/¹²CO₂ is used to determine the rate of gastric emptying after consumption of a standardized, ¹³C-enriched meal. Subjects will consume a standardized, ¹³C-enriched meal after an 8-hour fast at baseline (Days −10 to −3) and after an 8-hour fast starting 30 minutes after study drug administration on Day 14 (±2 days). Breath samples will be obtained twice prior to consumption of a standardized, ¹³C-enriched meal and then at 45, 90, 120, 150, 180, and 240 minutes after meal consumption on Days −10 to −3 and Day 14 (±2 days) and 30 minutes after study drug administration on Day 14 (±2 days).

### (ii) Secondary Efficacy Assessment

The secondary efficacy variables are the following:
The change from baseline in gastric emptying as measured by GEBT T_{1/2} (BT T_{1/2}) at Day 14 (±2 days)
The change from baseline in ANMS GCSI-DD total scores
The change from baseline in ANMS GCSI-DD subscale scores

The ANMS GCSI-DD covers 5 core relevant symptoms of gastroparesis: nausea, early satiety, postprandial fullness, upper abdominal pain and vomiting. Bloating is included as an exploratory symptom. Symptoms are rated on a severity numeric response scale from 0 (none) to 4 (very severe). Vomiting is captured on a frequency response scale and scored as follows: 0 for no episodes, 1 for one episode, 2 for 2 episodes, 3 for 3 episodes, and 4 for 4 or more episodes. Subjects will be asked to recall symptoms over the previous 24 hours and complete the ANMS GCSI-DD patient-reported symptom questionnaire for at least 14 days prior to randomization, on the day of randomization, and daily from Days 1 to 14 (±2 days). The total ANMS GCSI-DD score will be calculated as the mean of the 3 subscale scores for severity of nausea/vomiting, postprandial fullness/early satiety, and bloating. ANMS GCSI-DD subscale scores will be assessed separately. Baseline for analysis of the ANMS GCSI-DD will be based on the average score for the 3 days preceding randomization. Both total ANMS GCSI-DD and the subscale scores will be analyzed to compare symptom scores between the treatment groups for descriptive purposes only.

(iii) Efficacy Assessment

This efficacy variable is the change from baseline in gastroparesis symptoms as measured by PAGI-SYM questionnaire scores.

The PAGI-SYM includes 6 subscales: heartburn/regurgitation, fullness/early satiety, nausea/vomiting, bloating, upper abdominal pain, and lower abdominal pain. Both total and subscale PAGI-SYM scores will be used to compare symptom scores between the treatment groups for descriptive purposes only.

Clinical Grading Assessment Scales will also be included as an exploratory efficacy variable. For assessment of global clinical response, the following question will be utilized: “In thinking about the last 2 weeks, how would you say your stomach/gastroparesis-related problems/symptoms have been compared to the period before you started treatment on this study?” Responses will be: improved, no change, or worsened. Subjects will be asked to quantify their therapeutic response using the CPGAS. Subjects will pick a number over a range (+7=completely better; 0=no change; −7=very much worse) that best answers the question. Responders will be defined as CPGAS scores >0 and non-responders 0. Clinical Grading Assessment Scale scores will be compared between the deudomperidone-treated and placebo groups for descriptive purposes only.

### (iv) Pharmacokinetics

For all subjects, blood samples for PK analysis will be collected at the following visits and timepoints:
Day 1: within 60 minutes prior to and at 1 and 2 hour after the morning dose
Days 3 [±1 day], 7 [±1 day], and 10 [±1 day]: within 5 minutes prior to the morning dose
Day 14 [±2 days]: approximately 5 minutes prior to the morning dose and at 1, 2, 3, and 4 hours after the morning dose on
Day 15 or Day 16 (+1 day)

Subjects may consent to participate in a more intensive PK subgroup. In this subgroup, PK samples will also be collected as follows:
Day 1: at 3, 4, 6, 8, and 12 hours after the morning dose
Day 14 (±2 days): 6, 8, and 12 hours after the morning dose
Day 15
Day 16 (+1 day)

The following windows will be permitted for the collection of PK samples: ±2 minutes for samples collected 2 hours postdose and ±5 minutes for samples collected >2 hours postdose.

The following PK parameters will be calculated for deuterated domperidone (and any measured metabolites) using plasma concentrations measured following deudomperidone doses on Day 1 if the data permit:
Cₘₐₓ
Time to maximum plasma concentration
AUC 0-12 hours

The following PK parameters will be calculated for deuterated domperidone (and any measured metabolites) using plasma concentrations measured following the final dose of deudomperidone, if the data permit:
Cₘₐₓ
Time to maximum plasma concentration
AUC over a dosing interval
AUC from time 0 to the time of the last quantifiable plasma concentration
AUC from time 0 to infinity
Percent of AUC extrapolated
T_{1/2}
Apparent plasma clearance
Apparent volume of distribution

Trough concentrations collected throughout the study will be used to confirm compliance and attainment of steady-state. See, Table 1 for the schedules of procedures.

TABLE 1
Schedule of Procedures

| Baseline | Pre- | | | | | | | | | | | | | | | | Follow- |
|---|---|---|---|---|---|---|---|---|---|---|---|---|---|---|---|---|---|
| GEBT⁴ | Randomization | Treatment Period | | | | | | | | | | | | | | | up |
| Study Day | | | | | | | | | | | | | | | | | |
| −10 to −3 | −3 to −1 | 1 | 2 | 3 | 4 | 5 | 6 | 7 | 8 | 9 | 10 | 11 | 12 | 13 | 14 | 15⁶ | 16⁶ |

Visit Window (Days)

| | | | | |
|---|---|---|---|---|
| ±1 | ±1 | ±1 | ±2 | +1 |

PK Sampling Details

| | | | | | | |
|---|---|---|---|---|---|---|
| Multiple PK | X | | | | X | |
| sample¹ | | | | | | |
| Single PK | | X | X | X | | X^{2, 7} |
| sample² | | | | | | |

Procedure

| | | | | | | | | | | | | | | | | |
|---|---|---|---|---|---|---|---|---|---|---|---|---|---|---|---|---|
| GEBT³ | X | | | | | | | | | | | | | | | X |
| ANMS | X | X | X | X | X | X | X | X | X | X | X | X | X | X | X | X |
| GCSI-DD⁴ | | | | | | | | | | | | | | | | |
| PAGI-SYM⁴ | X | | | | | | | | | | | | | | | X |
| Clinical | | | | | | | | | | | | | | | | X |
| Grading | | | | | | | | | | | | | | | | |
| Assessment | | | | | | | | | | | | | | | | |
| Scales⁴ | | | | | | | | | | | | | | | | |
| PGx sample | | | | | | | | | | | | | | | | |

| | |
|---|---|
| (optional)⁵ | X |

¹On Day 1, blood samples for PK analysis will be collected from all subjects within 60 minutes prior to study drug administration and at approximately 1 and 2 hours post dose on Day 1. For subjects in the intensive PK subgroup, PK samples will also be collected at 3, 4, 6, 8, and 12 hours after the morning dose on Day 1 unless one or more of these timepoints is waived. Blood samples for PK analysis will be collected from all subjects within approximately 5 minutes prior to study drug administration and at 1, 2, 3, and 4 hours post dose on Day 14 (±2 days). For subjects in the intensive PK subgroup, PK samples will also be collected at 6, 8, and 12 hours after the morning dose on Day 14 (±2 days) unless one or more of these timepoints is waived. The following windows will be permitted for the collection of PK samples: ±2 minutes for samples collected ≤2 hours postdose and ±5 minutes for samples collected >2 hours postdose.
²On Days 3 (±1 day), 7 (±1 day), and 10 (±1 day), blood samples for PK analysis will be taken prior to the morning dose of study drug (within 5 minutes). For subjects in the intensive PK subgroup, PK samples will be collected on the day following their last dose of study drug (Day 15) and on Day 16 (+1 day). For subjects in the sparse PK subgroup, PK samples will be collected at Day 15 or Day 16 (+1 day).
³Subjects complete the ANMS GCSI-DD questionnaire daily at least 14 days prior to randomization. Questionnaires should be completed in the evening at approximately the same time each day. The PAGI-SYM questionnaire will be completed on Days −10 to − 3 prior to the start of the GEBT and Day 14 [±2 days] prior to the final dose of study drug. The Clinical Grading Assessment Scales will be completed on 14 [±2 days] prior to the start of the final dose of study drug and start of the GEBT.
⁴Sample collected predose. The Day 1 prolactin sample is not required to be repeated if a prolactin was obtained for the pre-randomization visit in the last 72 hours.
⁵For subjects participating in the optional PGx assessment, a blood sample will be collected at any time during the subject's participation in the study.
⁶For subjects participating in the intensive PK subgroup, PK samples will be collected on Day 15 and on Day 16 (+1 day). For subjects in the sparse PK subgroup, PK samples will be collected on Day 15 or Day 16 (+1 day).
⁷For subjects participating in the sparse PK subgroup and are only required to be complete either Day 15 or Day 16 (+1 day) these procedures will be performed on Day 16 (+1 day) if Day 15 was not performed.

### Example 2

This was a randomized, double-blind, placebo-controlled Phase 2a study to evaluate the safety, efficacy, PK, and dose response of oral deudomperidone in adults with idiopathic or diabetic gastroparesis.

The study began by randomizing subjects in Cohort 1 to either deudomperidone 10 mg BID or placebo BID in a ratio such that 15 subjects received deudomperidone and 5 subjects received placebo. Subjects opted into either a sparse PK sampling subgroup or an intensive PK sampling subgroup. Cohort 1 was stratified at randomization by idiopathic versus diabetic gastroparesis. Cohorts 2 and 3 were stratified by severity of gastroparesis: baseline gastric emptying breath test half-life (GEBT T½) 110 minutes (more severe gastroparesis) versus a baseline GEBT T½<110 minutes (less severe gastroparesis). Efforts were made to ensure adequate numbers of idiopathic and diabetic gastroparetic subjects were included.

After 16 subjects in Cohort 1 completed the study, an independent DRC convened to review the data from Cohort 1. The DRC reviewed emerging safety, PK, and/or efficacy data throughout the study to determine if it was safe and appropriate to continue the study with the planned dose levels for Cohorts 2 and 3 or if further refinement of the proposed dose levels was warranted. The highest dose was not to exceed 60 mg BID, and no more than 40 additional subjects were to be added without a protocol amendment. The DRC also determined to advance the cohorts serially.

Planned dose levels for Cohorts 2 and 3 were initially 30 mg BID and 60 mg BID, respectively, with 15 subjects receiving the active study drug and 5 subjects receiving placebo in each cohort. Based on emerging information from the current study as well as other ongoing studies, the planned dose levels and cohort sizes for the study were modified by the DRC as follows:
Cohort 1 was expanded to a total of 41 randomized subjects, and a 5 mg BID cohort (Cohort 3) was planned;
The dose level of Cohort 2 was changed to 20 mg once daily (QD), administered as a morning dose of a deudomperidone 20 mg capsule and an evening dose of a matching placebo capsule in order to maintain the study blind such that each dose may have involved active study drug or placebo; and
An expanded 15 mg BID cohort (Cohort 2 expansion) was planned and Cohort 3 was expanded; however, only Cohort 3 was enrolled.

At the Screening Visit, all subjects signed informed consent prior to any study procedures being performed. Subjects must have met all of the inclusion criteria and none of the exclusion criteria to be eligible for study participation.

Subjects were Evaluated as Follows:
Days −28 to −11: Screening procedures were performed. The ANMS GCSI-DD questionnaire was completed daily for at least 14 days prior to randomization (Day 1);
Days −10 to −3: The ANMS GCSI-DD questionnaire was completed daily. A GEBT was performed on a single day within this window, with breath samples obtained twice prior to consumption of a standardized, carbon-13 (¹³C)-enriched meal and then at 45, 90, 120, 150, 180, and 240 minutes after meal consumption after an 8-hour fast. Prior to the start of the GEBT, the baseline Patient Assessment of Gastrointestinal Disorders Symptom Severity Index (PAGI-SYM) was completed;
Days −3 to −1: A Pre-Randomization Visit was completed to perform safety assessments and to allow adequate turnaround time of laboratory results prior to randomization (Day 1); and
Days 1 to 14: Study drug was administered (deudomperidone or placebo BID) for 14 (±2) days and safety assessments were performed; the ANMS GCSI-DD questionnaire was completed daily and PK blood sampling was completed at specified time points. On Day 14 (±2 days), the PAGI-SYM and Clinical Grading Assessment questionnaires were completed prior to the final dose of study drug.
A GEBT was administered 30 minutes after study drug administration on Day 14 (±2 days). Breath samples were obtained twice prior to ¹³C-enriched meal consumption and then at 45, 90, 120, 150, 180, and 240 minutes after meal consumption after an 8-hour fast. If a subject withdrew from the study prior to the Day 14 (±2 days) GEBT, attempts should have been made to perform a GEBT on the last day of study drug administration. Clinical Grading Assessment Scales were also performed.

Subjects were stratified at randomization based on their etiology for gastroparesis (i.e., diabetic gastroparesis versus idiopathic gastroparesis) or, beginning in Cohort 2, by severity of gastroparesis using GEBT TK (i.e., more severe gastroparesis [a baseline GEBT T_{1/2}≥110 minutes] versus less severe gastroparesis [a baseline GEBT T_{1/2}<110 minutes]).

On days of the GEBT, subjects presented to the clinic after an overnight fast and should have taken their regular medications, including any treatment for diabetes, with the exception of any prohibited concomitant medication. Subjects self-administered their usual morning insulin injection, taking into consideration their fasting status. Subjects were instructed to bring in their study drug and additional insulin, as needed, to the clinic. Fasting blood glucose was assessed before study drug administration and gastric emptying assessments to ensure a blood glucose of ≤275 mg/dL. Additional insulin may have been given (prorated based on meal caloric content) to ensure a blood glucose of ≤275 mg/dL prior to the gastric emptying procedure. Likewise, Investigators should have managed low blood sugar (hypoglycemia; <60 mg/dL) as per their discretion.

Safety was evaluated through assessments of Aes, vital signs, physical examinations, clinical laboratory evaluations (including prolactin), and ECG findings. Additionally, a single, optional PGx blood sample may have been collected at any time during the subject's participation in the study.

Subjects had follow-up visit(s) 1 to 3 days after the final dose of study drug to assess Aes, changes to concomitant medications (including use of rescue medications), vital signs, and for PK sampling. Subjects participating in the intensive PK subgroup were seen in the clinic on Day 15 and Day 16 (+1 day). Subjects participating in the sparse PK subgroup had the option to complete either the Day 15 Visit or the Day 16 (+1 day) Visit. Unscheduled visits and/or additional follow-up may have been required. For example, subjects with clinically significant abnormal laboratory findings, unresolved TEAEs, SAEs that required follow-up laboratories and review, or clinically significant Aes may have necessitated further assessments.

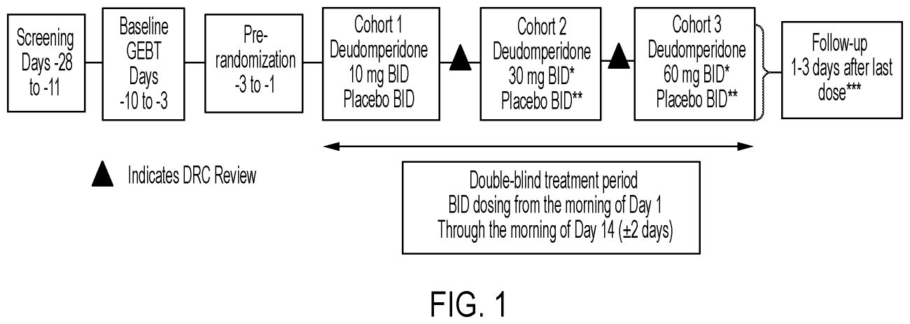

FIG. 1 presents the planned study schematic.

### A. Study Objectives

### Objectives

The objectives of this study were the following:
To evaluate the safety and tolerability of multiple doses of deudomperidone in subjects with idiopathic or diabetic gastroparesis;
To evaluate the effect of multiple doses of deudomperidone on gastric emptying as measured by the GEBT in subjects with idiopathic or diabetic gastroparesis; and
To characterize the PK of multiple doses of deudomperidone in subjects with idiopathic or diabetic gastroparesis.

### Exploratory Objectives

The exploratory objectives of this study included the following:
To assess the effect of multiple doses of deudomperidone on nausea associated with gastroparesis based on the nausea score on the ANMS GCSI-DD;
To assess the effect of multiple doses of deudomperidone on ANMS GCSI-DD scores (total and subscales) in subjects with idiopathic or diabetic gastroparesis;
To assess the effect of multiple doses of deudomperidone on a variety of PRO; and
To characterize exposure-response relationships of deudomperidone.

### B. Selection of Study Population

### Inclusion Criteria

Subjects who met all of the following criteria were eligible to participate in the study:
Male and female patients 18 to 70 years old;
Had a current diagnosis of idiopathic or diabetic gastroparesis as defined by the following:
   Had gastrointestinal symptoms felt to be consistent with gastroparesis (e.g., postprandial nausea/vomiting, postprandial fullness, early satiety, bloating, and/or epigastric/abdominal pain) within the 6 months prior to screening;
OR
   Had documented DGE within the past 3 years as determined by GEBT or scintigraphy. Patients who had DGE documented through other modalities (e.g., SmartPill, manometry, etc.) or who had an abnormal GEBT or scintigraphy >3 years ago may have been considered with Sponsor approval.
Had a BMI between 18 and 40 kg/m², inclusive;
Had glycosylated hemoglobin level ≤11% at screening;
Male patients with female partners of child-bearing potential must have agreed to use 2 medically accepted, highly effective methods of birth control from Day 1 through 60 days following the final dose of study drug;
   Medically accepted, highly effective methods of birth control for male patients with female partners of child-bearing potential included the following: latex condom with spermicide, diaphragm with intravaginal spermicide, cervical cap with spermicide, indwelling intrauterine device (hormonal or nonhormonal), implanted contraceptives, and oral contraceptives.
Male patients must have agreed to abstain from sperm donation from Day 1 through 60 days following the final dose of study drug;
Female patients with male partners must have been surgically sterile (hysterectomy and/or bilateral oophorectomy), postmenopausal for at least 1 year (with confirmed FSH in postmenopausal range at the Screening Visit), or agreed to use 2 medically accepted, highly effective methods of birth control from Day −14 until 60 days following the final dose of study drug—medically accepted, highly effective methods of birth control for female patients with male partners included the following: latex condom with spermicide, diaphragm with intravaginal spermicide, cervical cap with spermicide, and nonhormonal indwelling intrauterine device.
Had a negative breath alcohol and urine drug screen (including tetrahydrocannabinol) at the Screening Visit and at the time of randomization;
Was willing to abstain from grapefruit, grapefruit products, star fruit, star fruit products, and Seville oranges from 48 hours prior to randomization until end of study;
Was willing to refrain from long-acting GLP-1 agonists, sodium-glucose transporter-2 (SGLT-2) inhibitors, or pramlintide; and Subjects who were taking SGLT-2 inhibitors or GLP-1 agonists other than Bydureon (exenatide extended release), Tanzeum (albiglutide), or Trulicity (dulaglutide) may have been considered for the study if they were willing to discontinue the medication(s) for 3 days prior to each GEBT Visit (Days −10 to −3 and Day 14 [±2 days]).
Was able to understand and willing to comply with all study visits, procedures, restrictions, and discontinuation of medications, including those to treat gastroparesis, and provide written informed consent according to institutional and regulatory guidelines.

### Exclusion Criteria

Subjects who met any of the following criteria were excluded from participation in the study:
1. Had a history of, or current, clinically significant arrhythmias, including ventricular tachycardia, ventricular fibrillation, and Torsades de pointes. Patients with minor forms of ectopy (e.g., premature atrial contractions) were not necessarily excluded;
2. Had clinically significant bradycardia with a resting heart rate under 50 beats per minute, sinus node dysfunction, or heart block;
3. Had prolonged QTcF (QTcF >450 msec for males or QTcF >470 msec for females) based on the average of triplicate ECGs;
4. Had a personal or family history of long QT syndrome, Torsades de pointes, or other complex ventricular arrhythmias or family history of sudden death;
5. Had evidence (based on screening or baseline assessments) or history of clinically significant immunologic, hematologic, renal, endocrine, pulmonary, gastrointestinal, cardiovascular, hepatic, psychiatric, neurologic, or allergic disease (including drug allergies); surgical conditions; cancer (with the exception of basal or squamous cell carcinoma of the skin and cancer that resolved or has been in remission for >5 years prior to the Screening Visit); or any condition that might have significantly interfered with the absorption, distribution, metabolism, or excretion of the study drug; Cholecystectomy and appendectomy were allowed.
6. Had a history of prolactin-releasing pituitary tumor (i.e., prolactinoma);
7. Had elevated serum prolactin—Patients who were on medications that are known to increase prolactin (e.g., domperidone, metoclopramide, phenothiazines, selective serotonin reuptake inhibitors, verapamil, etc.) with an elevated prolactin at the time of screening may have been considered for this study.
8. Had known or suspected hypogonadism, current clinically significant menstrual abnormalities (e.g., oligomenorrhea or amenorrhea), gynecomastia, galactorrhea, or other clinical features that may have been consistent with hyperprolactinemia;
9. Had pyloric injection of botulinum toxin within 6 months of screening and/or during the course of the study;
10. Had bilirubin, alkaline phosphatase, AST, or ALT levels greater than 2×ULN or had a Child-Pugh classification grade of B or C;
11. Had a serum creatinine level greater than 1.5×ULN or had an eGFR <30 mL/min/1.73 m² using the CKD-EPI equation at screening;
12. Had a hemoglobin level <10 g/dL at screening;
13. Had thyroid-stimulating hormone levels that were abnormal at screening;
14. Had an inability to perform a breath test;
15. Had a known allergy to eggs or spirulina;
16. Used investigational, prescription, or over-the-counter medications as follows:
   Active participation in an experimental therapy study; received experimental therapy with a small molecule within 30 days of randomization or 5 half-lives (whichever was longer); or received experimental therapy with a large molecule within 90 days of randomization or 5 half-lives, (whichever was longer);
   Use of a concomitant CYP3A4 inhibitor (whether a medication, herbal supplement, dietary supplement, or nutraceutical) within 14 days or 5 half-lives (whichever was longer) of the first dose of study drug until the end of the study;
   Use of a concomitant CYP3A4 inducer (whether a medication, herbal supplement, dietary supplement, or nutraceutical) within 28 days or 5 half-lives (whichever was longer) of the first dose of study drug or inducer until the end of the study;
   Use of any medication, herbal supplements, dietary supplements, or nutraceuticals with potential to prolong QT within 28 days prior to the first dose of study drug until the end of the study; or
   Use of motility agents (including, but not limited to, metoclopramide, domperidone, erythromycin, etc.), other medications that may have affected gastric emptying (e.g., opiates, marijuana, and anticholinergics), and antiemetics (except for protocol-specified rescue antiemetic medication) within 14 days prior to baseline GEBT until the end of the study. Gastric pacemakers should have been turned off within 14 days prior to baseline GEBT and kept off until end of study.
17. History of alcoholism or drug abuse within 2 years prior to dosing as defined by the Diagnostic and Statistical Manual of Mental Disorders, 4^{th} Edition;
18. Typical consumption of ≥14 alcoholic drinks weekly;
19. Evidence of ongoing illicit drug use;
20. Positive for HIV antibody, HCV by RNA, or HbsAg at the Screening Visit;
21. Donated blood or blood products within 30 days prior to dosing;
22. Was currently undergoing treatment with weight loss medication or prior weight loss surgery (e.g., gastric bypass surgery);
23. Was pregnant, breastfeeding, or planning to become pregnant during the course of study;
24. Had an inability to swallow medication;
25. Was currently receiving parenteral feeding or presence of a nasogastric or other enteral tube (e.g., PEG tube) for feeding or decompression;
26. Known or suspected gastric outlet obstruction (e.g., peptic stricture) or other gastrointestinal mechanical obstruction as documented by upper gastrointestinal tract endoscopy or upper gastrointestinal radiographic series in the past 3 years;
27. Known history or current diagnosis of intestinal malabsorption or pancreatic exocrine disease;
28. History of gastric surgery such as fundoplication, gastrectomy, vagotomy, pyloroplasty, or bariatric procedure; Patients who had a gastric pacemaker in place may have been considered for the study if the pacemaker could be turned off for at least 14 days prior to baseline GEBT and throughout the remainder of the study. A history of diagnostic endoscopy was not exclusionary. Cholecystectomy and appendectomy were allowed.
29. History or presence of any medical condition or psychiatric disease, which could have interfered with the conduct of the study or would have put the patient at unacceptable risk;
30. Prior lack of response to domperidone or known hypersensitivity or intolerance to domperidone or any of the excipients in the deudomperidone formulation; or
31. Unsuitable for any other reason that may have either placed the patient at increased risk during participation or interfered with the interpretation of the study outcomes.

### C. Randomization Criteria

In addition to the general criteria, in order to be randomized into the study, subjects must have also met the following criteria at the time of randomization:
1. Continued to satisfy all inclusion/exclusion criteria;
2. Had been off all motility agents and antiemetics for at least 14 days and was willing to remain off such medications (except for protocol-specified antiemetic rescue medication) during the course of the study;
3. Demonstrated a confirmed diagnosis of idiopathic or diabetic gastroparesis as defined by the following:
Current upper gastrointestinal symptoms (e.g., nausea/vomiting, postprandial fullness, early satiety, bloating, and/or epigastric/abdominal pain); or
Documented DGE within the 10 days prior to first dose of study drug based on GEBT using ¹³C-Spirulina platensis demonstrating T½>80 minutes. Subjects with a T½>200 minutes may have been considered for the study with Sponsor approval.
4. Had an ANMS GCSI-DD score that satisfied the following:
An average composite score of ≥2 (utilizing a Likert scale from 0 [none] to 4 [very severe]) for the last 10 days prior to randomization or approval by the Sponsor. The predominant symptom experienced should not have been abdominal pain; and
A nausea subscale score (comprising nausea, retching, and vomiting) ≥2 at least 4 of 7 days; OR at least 1 episode of vomiting during the portion of the Screening Period during which the subject had not been taking motility agents or antiemetics (with the exception of protocol-specified rescue medication).
5. Adherence with the ANMS GCSI-DD during the Screening Period, defined as adherence ≥75% or approval by the Sponsor; and
6. Had continued required results of the initial safety laboratory test performed at screening.

### Removal of Subjects From Therapy or Assessment

Participation of a subject in this clinical study may have been discontinued for any of the following reasons:
Subject withdrew consent or requested discontinuation from the study for any reason;
Occurrence of any medical condition or circumstance that exposed the subject to substantial risk and/or did not allow the subject to adhere to the requirements of the study protocol;
Any SAE, clinically significant AE, severe laboratory abnormality, clinically significant change in ECG from baseline (e.g., QTcF >500 msec, change in QTcF >60 msec from baseline [confirmed by repeat ECG with central interpretation], etc.), new ECG abnormality deemed clinically significant, intercurrent illness, or other medical condition that indicated that continued participation was not in the best interest of the subject;
Pregnancy;
Requirement of prohibited concomitant medication;
Subject failure to comply with study protocol requirements or study-related procedures; or
Termination of the study by the Sponsor or the regulatory authority.

If a subject withdrew prematurely from the study due to the above criteria or any other reason, study staff should have made every effort to complete the full panel of assessments scheduled for the Early Termination Visit. The reason for subject withdrawal must have been documented.

Study enrollment was to continue until 60 subjects (15 subjects per treatment group: deudomperidone 10 mg BID, deudomperidone 20 mg QD, deudomperidone 5 mg BID, and Placebo) had completed the treatment period. The DRC could have expanded study enrollment, but no more than 40 additional subjects were to be added without a protocol amendment. Withdrawn subjects were not replaced.

### D. Treatments

### (i) Treatments Administered

Subjects were dosed with either deudomperidone or placebo capsules orally. Subjects were randomized to either deudomperidone or placebo in a 3:1 ratio.

The doses of deudomperidone administered to Cohorts 1 through 3 were 10 mg BID, 20 mg QD, and 5 mg BID. A Cohort 2 expansion at a dose level of 15 mg BID was approved by the DRC, but no subjects were enrolled. All subjects took a dose of blinded study drug (deudomperidone or placebo capsules) orally at 8:00 AM (±2 hours) and 8:00 PM (±2 hours) with approximately 240 mL of water. A 12-hour interval was maintained between each dose for a given individual. All subjects should have made an effort to fast for a minimum of 8 hours before the morning dose on Day 1.

The duration of the double-blind treatment period was 14 (±2) days of study drug administration.

### (ii) Identity of Products

Deudomperidone capsules consisted of deudomperidone drug product in a size 2C, oval-shaped, blue, softgel capsule. Each deudomperidone capsule contained either 5, 10, or 20 mg of active deudomperidone drug substance (deuterated domperidone). The deudomperidone capsules contained the ingredients presented in Table 1.

TABLE 2
Composition of Deudomperidone Capsules
Component

Deudomperidone (5, 10, or 20 mg)
Medium chain triglycerides
Glyceryl distearate
Butylated hydroxyanisole
Butylated hydroxytoluene
Softgel capsule ¹
¹ The softgel capsule contained the following ingredients: gelatin 150 LB, NF; glycerin, USP; gelatin hydrolysate, NF; purified water, USP; titanium dioxide, USP; and FD&C blue #1.

A matching placebo softgel capsule containing all the components in Table 1, with the exception of deudomperidone drug substance, was also provided. The softgel capsules (deudomperidone and placebo) were packaged in high-density polyethylene bottles with child-resistant screw cap closures or blister packs. Lot numbers for the study drugs used in this study are listed in Table 3.

TABLE 3
Study Drugs

| Study Drugs | Strength and Mode of Administration |
|---|---|
| Deudomperidone | 5 mg; orally |
| | 10 mg; orally |
| | 20 mg; orally |
| Placebo | 0 mg matching capsules; orally |

Study drug (deudomperidone and placebo) was stored at ambient room temperature (15 to 25° C. [59 to 77° F.]), away from moisture and light.

(iii) Method of Assigning Subjects to Treatment Groups

This study planned to include 3 treatment groups with 20 subjects each. The study began by randomizing subjects in Cohort 1 to either deudomperidone 10 mg BID or placebo BID in a ratio such that 15 subjects received deudomperidone and 5 subjects received placebo. An independent DRC reviewed emerging safety, PK, and/or efficacy data throughout the study to determine if it was safe and appropriate to continue the study with the planned dose levels for Cohorts 2 and 3 or if further refinement of the proposed dose levels was warranted. Planned dose levels for Cohorts 2 and 3 were initially 30 mg BID and 60 mg BID, respectively, with 15 subjects receiving study drug and 5 subjects receiving placebo in each cohort. Based on emerging information from the current study as well as other ongoing studies, the planned dose levels and cohort sizes for the study were modified as follows:
Cohort 1 was expanded to a total of 41 randomized subjects, and a 5 mg BID cohort (Cohort 3) was planned;
The dose level of Cohort 2 was changed to 20 mg QD, administered as a morning dose of a deudomperidone 20 mg capsule and an evening dose of a matching placebo capsule in order to maintain the study blind such that each dose may have involved active study drug or placebo; and
Cohort 2 expansion to 18 subjects was planned at a dose level of 15 mg BID consisting of a 10 mg and a 5 mg capsule taken as a single dose. Due to changes in manufacturing methods of the 10 mg capsules, the Sponsor opted not to move forward with the 15 mg BID dose level to avoid introducing potential variability between lots and thus confounding data analysis. The 5 mg BID (Cohort 3) was expanded to a total of 18 subjects, but only 12 subjects were randomized.

Subjects who completed the Screening Visit and met all of the inclusion criteria, none of the exclusion criteria, and all of the additional criteria based on randomization procedures, including safety laboratory tests, were randomized into the study. Subjects were randomized to either deudomperidone BID or placebo BID in a 3:1 ratio. Randomization occurred on Day 1.

### (iv) Selection and Timing of Dose for Each Subject

All subjects took a dose of blinded study drug (deudomperidone or placebo capsules) orally at 8:00 AM (±2 hours) and 8:00 PM (±2 hours) with approximately 240 mL of water. A 12-hour interval was maintained between each dose for a given individual. All subjects should have made an effort to fast for a minimum of 8 hours before the morning dose on Day 1. However, alight meal may have been consumed >2 hours prior to the morning dose of study drug if determined that food should be consumed by the subject (i.e., due to low blood glucose levels or symptoms of hypoglycemia). Subjects providing sparse PK samples should have continued to fast for a minimum of 2 hours and subjects providing more intensive PK samples should have continued to fast for a minimum of 4 hours after the morning dose on Day 1. However, if determined that food should be consumed, it was permissible to break the specified fast to ensure subject safety. On Day 14 (±2 days), all subjects fasted for a minimum of 8 hours before the morning dose of study drug and 4 hours following the completion of the GEBT meal. Subjects were required to fast 2 hours before and 1 hour after all other doses. Subjects were provided study drug for self-administration for doses not administered at the site.

In the event that a subject missed their scheduled dose and became aware of the missed dose within 2 hours of their scheduled dosing time, the subject was instructed to take their assigned dose of study drug at the time they realized that the dose was missed. If the subject became aware of the missed dose more than 2 hours after their scheduled dosing time, the subject was instructed to skip this dose of study drug and resume the assigned dose of study drug at the next scheduled time.

In the event that the subject vomited after taking their scheduled dose of study drug, subjects were instructed to note the time of study drug administration and time of vomiting and to resume their assigned dose of study drug at the next scheduled time. During the treatment period, subjects should have attempted to note the time of vomiting in relation to time of study drug administration and report this to site staff during scheduled clinic visits.

### (v) Blinding

Following randomization, study drug was dispensed in a double-blind manner on Day 1. The Sponsor and all clinical personnel were blinded to the treatment group for each subject. Subjects were also blinded to the treatment they received.

Subjects were stratified at randomization based on their etiology for gastroparesis (i.e., diabetic gastroparesis versus idiopathic gastroparesis) or, beginning in Cohort 2, by severity of gastroparesis using GEBT T½ (i.e., more severe gastroparesis [a baseline GEBT T½>110 minutes] versus less severe gastroparesis [a baseline GEBT T½<110 minutes]).

Bioanalytical staff involved in analysis of PK samples were unblinded to treatment by the nature of the results of sample analysis. The PK data were de-identified before being provided to any other individuals, including those involved in calculating PK parameters and associated descriptive statistics, performing any modelling or simulations, and/or plotting PK data, in order to maintain blinding.

### (vi) Prior and Concomitant Therapy

Excluded Medications, Foods, and/or Procedures

Use of the following investigational, prescription, or over-the-counter medications was not permitted during the study:
Pramlintide;
SGLT-2 inhibitors or GLP-1 agonists with long half-lives (e.g., Bydureon [exenatide extended release], Tanzeum [albiglutide], or Trulicity [dulaglutide]);
Experimental therapy with a small molecule within 30 days prior to randomization or 5 half-lives (whichever was longer); or experimental therapy with a large molecule within 90 days prior to randomization or 5 half-lives (whichever was longer);
CYP3A4 inhibitors (whether a medication, herbal supplement, dietary supplement, nutraceutical, or food) within 14 days or 5 half-lives (whichever was longer) of the first dose of study drug or inducer until the end of the study;
CYP3A4 inducers (whether a medication, herbal supplement, dietary supplement, nutraceutical, or food) within 28 days or 5 half-lives (whichever was longer) of the first dose of study drug until the end of the study;
Medication, herbal supplements, dietary supplements, or nutraceuticals with potential to prolong QT within 28 days prior to the first dose of study drug until the end of the study;
Motility agents (including, but not limited to, metoclopramide, domperidone, erythromycin, etc.), other medications that may have affected gastric emptying (e.g., opiates, marijuana, and anticholinergics), and antiemetics (except for protocol-specified rescue antiemetic medication) within 14 days prior to baseline GEBT until the end of the study; Gastric pacemakers should have been turned off within 14 days prior to baseline GEBT and kept off until end of study.
Pyloric injection of botulinum toxin within 6 months of screening and during the course of the study;
Weight loss medication or prior weight loss surgery (e.g., gastric bypass surgery); and
Parenteral feeding or presence of a nasogastric or other enteral tube (e.g., PEG tube) for feeding or decompression.

Rescue medications: Subjects who experienced severe symptoms of gastroparesis may have received a single dose of promethazine hydrochloride (Phenergan®) per day but should have avoided such treatment, if possible, on the days of GEBT. Subjects who required further treatment with prohibited medications may have been discontinued from study treatment and undergone follow-up study procedures.

Allowed medications and/or procedures: Other medications that were not explicitly and previously excluded were permitted (e.g., rescue medications).

Documentation of prior and concomitant medication use: Any medications administered 28 days prior to the first dose of study drug and/or during the study period must have been recorded.

### E. Efficacy and Safety Variables

### (i) Efficacy and Safety Measurements Assessed and Flow Chart

Tables 4A and 4B provide the Schedule of Procedures for the study.

TABLE 4A
Schedule of Procedures

| | | Baseline | Pre- |
|---|---|---|---|
| | Screening | GEBT ¹⁶ | Randomization |
| Study Day | −28 to −11 | −10 to −3 | −3 to −1 |
| Visit Window (Days) | | | |
| Clinic Visit Day | X | X | X |

Procedure

| Informed consent ³ | X | | |
|---|---|---|---|
| I/E criteria ⁴ | X | | X |
| Randomization criteria | | | |
| Demographics | X | | |
| Medical/surgical history | X | | X |
| Prior and concomitant | X | | |
| Medications ⁵ | | | |
| Adverse events ⁶ | | | |
| Complete physical | X | | |
| Examination ⁷ | | | |
| Weight and height ⁸ | X | | |
| Vital signs ⁹ | X | X | X |
| Breath alcohol test | X | | |
| Urine drug screen | X | | X |
| Urinalysis | X | | X |
| HIV/HbsAg/HCV | X | | |
| Clinic Visit Day | X | X | X |
| FSH ¹⁰ | X | | |
| TSH | X | | |
| Pregnancy test ¹² | X | | X ¹² |
| 12-lead ECG ¹³ | X | | |
| GEBT ¹⁵ | | X | |
| ANMS GCSI-DD ¹⁶ | X | X | X |
| PAGI-SYM ¹⁶ | | X | |
| Chemistry, hematology, and | X | | X |
| coagulation | | | |
| Glycosylated hemoglobin | X | | |
| Prolactin ¹⁷ | X | | X |
| Clinic Visit Day | X | X | X |

| | |
|---|---|
| PGx sample (optional) ²¹ | X |

All days were reported as the next nominal day in sequential order (i.e., if the outpatient visit Day 3 [±1 day] took place on the sequential Day 4, it was recorded as Day 4. All following days were recorded likewise).
¹ On Day 1, all subjects were confined to the clinic for a minimum of approximately 2 hours after the morning dose of study drug. Blood samples for PK analysis were collected from all subjects within 60 minutes prior to study drug administration and at approximately 1 and 2 hours post-dose on Day 1. Subjects in the intensive PK subgroup remained confined to the clinic and PK samples were also collected at 3, 4, 6, 8, and 12 hours after the morning dose on Day 1 unless 1 or more of these time points was waived in writing by the Sponsor ahead of time. On Day 14 (±2 days), subjects were confined in the clinic for a minimum of approximately 4 hours after the morning dose of study drug. Blood samples for PK analysis were collected from all subjects within approximately 5 minutes prior to study drug administration and at 1, 2, 3, and 4 hours post-dose on Day 14 (±2 days). Subjects in the intensive PK subgroup remained confined to the clinic and PK samples were also collected at 6, 8, and 12 hours after the morning dose on Day 14 (±2 days) unless 1 or more of these time points was waived in writing by the Sponsor ahead of time. The following windows were permitted for the collection of PK samples: ±2 minutes for samples collected ≤2 hours post-dose and ±5 minutes for samples collected >2 hours post-dose.
² On Days 3 (±1 day), 7 (±1 day), and 10 (±1 day), all subjects reported to the clinic for an outpatient visit. Blood samples for PK analysis were taken prior to the morning dose of study drug (within 5 minutes). Subjects in the intensive PK subgroup reported to the clinic on the day following their last dose of study drug (Day 15) and on Day 16 (±1 day) and a PK sample was collected at each visit. Subjects in the sparse PK subgroup may have reported to the clinic on either Day 15 or Day 16 (±1 day) and a PK sample was collected at this visit.
³ Written informed consent for the study was obtained from all subjects before any study procedures were performed.
⁴ Screening procedures, including vital sign assessments, may have been repeated no more than 2 times for eligibility purposes.
⁵ Any medications administered 28 days prior to the first dose of study drug and during the study period must have been recorded. Starting on Day 1, concomitant medications were recorded by the subject prior to each dose and recorded at each outpatient visit prior to each dose.
⁶ Aes were monitored and documented at each outpatient visit from the time first dose of study drug until study participation was complete.
⁷ A complete physical examination consisted of general appearance, skin, head, eyes, ears, mouth, oropharynx, neck, heart, lungs, abdomen, extremities, and neuromuscular system. A limited physical examination consisted of a minimum of general appearance, skin, heart, lungs, and abdomen.
⁸ Height was measured at the Screening Visit only. BMI was calculated at the Screening Visit using height and weight collected at the Screening Visit. At the time of randomization (Day 1), BMI was calculated using weight from the time of randomization (Day 1) and height from the Screening Visit.
⁹ Vital signs included heart rate, blood pressure, respiration rate, and temperature and were collected while the subject was sitting after a minimum 5-minute rest.
¹⁰ FSH was only measured for females who had been postmenopausal for at least 1 year and were not surgically sterile.
¹¹ At the time of randomization (Day 1), a POC drug test was performed for use in assessing eligibility. A urine sample was also sent to the central lab for confirmatory testing.
¹² A pregnancy test was only performed in females who were not surgically sterile (or postmenopausal for at least 1 year [with confirmed FSH in postmenopausal range at the Screening Visit]). A serum pregnancy test was performed by the central laboratory on Days −28 to −11 and Day 14 (±2 days). A POC pregnancy test was initially performed on the Day −3 to −1 visit for use in assessing randomization eligibility.
¹³ All 12-lead ECGs were performed in triplicate approximately 1 minute apart after the subject had been resting in the supine position for at least 10 minutes. Twelve-lead ECGs were printed and interpreted as soon as possible. A digital recording of all ECGs from randomized subjects was submitted to a central reviewer. ECGs should have been completed ±15 minutes from all specified time points.
14. On Days 1 and 14 (±2 days), ECGs were performed prior to the morning dose and at 2, 4, 6, and 12 hours (as applicable) after the morning dose and should have been completed +15 minutes from the specified time points. For subjects participating in the intensive PK, additional ECGs were performed at 4, 6, and 12 hours (if applicable) after the morning dose). If collection of specific individual PK samples (including, but not limited to, the samples at 8 and/or 12 hours post-dose for the intense PK subgroup) were not required based on written Sponsor approval, the final ECGs for these visits must have been collected when the final PK sample was obtained.
¹⁵ Subjects had baseline GEBT performed between Days −10 and −3 after a minimum 14-day washout from motility agents and on Day 14 (±2 days) 30 minutes after study drug administration. Subjects fasted for a minimum of 8 hours before the breath test. Breath samples were obtained twice prior to consumption of a standardized, ¹³C-enriched meal and then on Days −10 to −3 at 45, 90, 120, 150, 180, and 240 minutes post meal after an 8-hour fast and on Day 14 (±2 days) at 45, 90, 120, 150, 180, and 240 minutes starting 30 minutes after study drug administration. Fasting glucose was assessed before GEBT to ensure a blood glucose level of ≤275 mg/dL.
¹⁶ Subjects were instructed to complete the ANMS GCSI-DD questionnaire daily at least 14 days prior to randomization (Day 1). Materials were dispensed at the Screening Visit. Adherence to ANMS GCSI-DD was assessed at the time of randomization (Day 1) and must have been ≥75% to be eligible for the study. Questionnaires should have been completed in the evening at approximately the same time each day. The PAGI-SYM questionnaire was completed at the study site on Days −10 to −3 prior to the start of the GEBT and on Day 14 (±2 days) prior to the final dose of study drug. The Clinical Grading Assessment Scales were completed on Day 14 (±2 days) prior to the start of the final dose of study drug and start of the GEBT.
¹⁷ Sample collected pre dose. The Day 1 prolactin sample was not required to be repeated if a prolactin sample was obtained for the Pre-Randomization Visit in the last 72 hours.
18. Study drug was dispensed with treatment assigned on Day 1.
19. All subjects took a dose of blinded study drug (deudomperidone or placebo capsules) orally at 8:00 AM (±2 hours) and 8:00 PM (±2 hours) with approximately 240 mL of water. A 12-hour interval was maintained between each dose for a given individual. Fasting requirement for Day 1: Subjects participating in the intensive PK subgroup should have made an effort to fast for a minimum of 8 hours before the morning dose of study drug. However, a light meal may have been consumed >2 hours prior to the morning dose of study drug if determined that food should be consumed (i.e., due to low blood glucose levels or symptoms of hypoglycemia). Subjects should have made an effort to fast for 4 hours after the morning dose of study drug; however, if determined that food should be consumed, it was permissible to break the specified fast to ensure subject safety. For the evening dose, subjects fasted for >2 hours before and 1 hour after study drug. The evening dose was self-administered by the subject. Subjects participating in the sparse PK subgroup should have made an effort to fast for a minimum of 8 hours before the morning dose of study drug. However, a light meal may have been consumed >2 hours prior to the morning dose of study drug if determined that food should be consumed by the subject (i.e., due to low blood glucose levels or symptoms of hypoglycemia). Subjects should have fasted for >2 hours after the morning dose of study drug; however, if determined that food should be consumed, it was permissible to break the specified fast to ensure subject safety. For the evening dose, subjects fasted for a minimum of 2 hours before and 1 hour after the dose of study drug. The evening dose was self-administered by the subject. If a subject reported vomiting during the treatment period, subjects should have attempted to note the time of vomiting in relation to time of study drug administration and report this to site staff during scheduled clinic visits.
20. Study drug accountability occurred on Days 3, 7, 10, and 14 or on Early Termination if applicable. Unused study drug was only collected on Day 14 (±2 days) or on Early Termination if applicable.
²¹ For subjects who provided written informed consent to participate in the optional PGx assessment, a blood sample was collected at any time during the subject's participation in the study.
22. Subjects participating in the intensive PK subgroup reported to the clinic on Day 15 and on Day 16 (±1 day) where a PK sample was collected at each respective visit. Subjects in the sparse PK subgroup may have reported to the clinic on either Day 15 or Day 16 (±1 day) and a PK sample was collected at the selected visit.
23. For subjects participating in the sparse PK subgroup and who were only required to complete either Day 15 or Day 16 (±1 day), these procedures were performed on Day 16 (±1 day) if Day 15 was not performed.

TABLE 4B
Schedule of Procedures

| | | | | | | | | | | | | | | | Follow- |
|---|---|---|---|---|---|---|---|---|---|---|---|---|---|---|---|
| Treatment Period | | | | | | | | | | | | | | | Up |
| Study Day | | | | | | | | | | | | | | | |
| 1 | 2 | 3 | 4 | 5 | 6 | 7 | 8 | 9 | 10 | 11 | 12 | 13 | 14 | 15²² | 16²² |

| | Visit Window (Days) | | | | | | |
|---|---|---|---|---|---|---|---|
| | | ±1 | ±1 | ±1 | ±2 | | ±1 |
| Clinic Visit | X | X | X | X | X | X | X |
| Day | | | | | | | |

PK Sampling Details

| | | | | | | |
|---|---|---|---|---|---|---|
| Multiple PK | X | | | | X | |
| sample ¹ | | | | | | |
| Single PK | | X | X | X | | X ^{2, 23} |
| sample ² | | | | | | |

Procedure

| Informed | | | | | | | | | | | | | | | | |
|---|---|---|---|---|---|---|---|---|---|---|---|---|---|---|---|---|
| consent ³ | | | | | | | | | | | | | | | | |
| I/E criteria ⁴ | X | | | | | | | | | | | | | | | |
| Randomization | X | | | | | | | | | | | | | | | |
| criteria | | | | | | | | | | | | | | | | |
| Medical/ | X | | | | | | | | | | | | | | | |
| surgical history | | | | | | | | | | | | | | | | |
| Prior and | X | | | | | | | | | | | | | | | |
| concomitant | | | | | | | | | | | | | | | | |
| Medications ⁵ | | | | | | | | | | | | | | | | |
| Adverse events ⁶ | X | | | | | | | | | | | | | | | |
| Complete | | | | | | | | | | | | | | X | | |
| physical | | | | | | | | | | | | | | | | |
| examination ⁷ | | | | | | | | | | | | | | | | |
| Limited | X | | | | | | | | | | | | | | | |
| physical | | | | | | | | | | | | | | | | |
| examination ⁷ | | | | | | | | | | | | | | | | |
| Weight and | X ⁸ | | | | | | | | | | | | | | | |
| height ⁸ | | | | | | | | | | | | | | | | |
| Vital signs ⁹ | X | | X | | | | X | | | X | | | | X | X²³ | X |
| Breath alcohol | X | | | | | | | | | | | | | | | |
| test | | | | | | | | | | | | | | | | |
| Urine drug | X¹¹ | | | | | | | | | | | | | X | | |
| screen | | | | | | | | | | | | | | | | |
| Urinalysis | | | X | | | | X | | | X | | | | X | | |
| Clinic Visit | X | | X | | | | X | | | X | | | | X | X | X |
| Day | | | | | | | | | | | | | | | | |
| Pregnancy test | | | | | | | | | | | | | | X | | |
| ¹² | | | | | | | | | | | | | | | | |
| 12-lead ECG ¹³ | X¹⁴ | | X | | | | X | | | X | | | | X¹⁴ | X²³ | |
| GEBT ¹⁵ | | | | | | | | | | | | | | X | | |
| ANMS GCSI- | X | X | X | X | X | X | X | X | X | X | X | X | X | X | | |
| DD ¹⁶ | | | | | | | | | | | | | | | | |
| PAGI-SYM ¹⁶ | | | | | | | | | | | | | | X | | |
| Clinical | | | | | | | | | | | | | | X | | |
| Grading ¹⁶ | | | | | | | | | | | | | | | | |
| Chemistry, | | | X | | | | X | | | X | | | | X | | |
| hematology, | | | | | | | | | | | | | | | | |
| and | | | | | | | | | | | | | | | | |
| coagulation | | | | | | | | | | | | | | | | |
| Prolactin ¹⁷ | X¹⁷ | | X | | | | X | | | X | | | | X | | |
| Randomize | X | | | | | | | | | | | | | | | |
| subject | | | | | | | | | | | | | | | | |
| Dispense study | X | | | | | | | | | | | | | | | |
| drug ¹⁸ | | | | | | | | | | | | | | | | |
| Study drug | X | X | X | X | X | X | X | X | X | X | X | X | X | X | | |
| administration | | | | | | | | | | | | | | | | |
| ¹⁹ | | | | | | | | | | | | | | | | |
| Study drug | | | X | | | | X | | | X | | | | X | | |
| accountability | | | | | | | | | | | | | | | | |
| and collection | | | | | | | | | | | | | | | | |
| of unused | | | | | | | | | | | | | | | | |
| study drug ²⁰ | | | | | | | | | | | | | | | | |
| Clinic Visit | X | | X | | | | X | | | X | | | | X | X | X |
| Day | | | | | | | | | | | | | | | | |
| Collection of | | | | | | | | | | | | | | | X²³ | |
| electronic diary | | | | | | | | | | | | | | | | |
| materials | | | | | | | | | | | | | | | | |
| PGx sample | X | | | | | | | | | | | | | | | |
| (optional) ²¹ | | | | | | | | | | | | | | | | |

All days were reported as the next nominal day in sequential order (i.e., if the outpatient visit Day 3 [±1 day] took place on the sequential Day 4, it was recorded as Day 4. All following days were recorded likewise).
¹ On Day 1, all subjects were confined to the clinic for a minimum of approximately 2 hours after the morning dose of study drug. Blood samples for PK analysis were collected from all subjects within 60 minutes prior to study drug administration and at approximately 1 and 2 hours post-dose on Day 1. Subjects in the intensive PK subgroup remained confined to the clinic and PK samples were also collected at 3, 4, 6, 8, and 12 hours after the morning dose on Day 1 unless 1 or more of these time points was waived in writing by the Sponsor ahead of time. On Day 14 (±2 days), subjects were confined in the clinic for a minimum of approximately 4 hours after the morning dose of study drug. Blood samples for PK analysis were collected from all subjects within approximately 5 minutes prior to study drug administration and at 1, 2, 3, and 4 hours post-dose on Day 14 (±2 days). Subjects in the intensive PK subgroup remained confined to the clinic and PK samples were also collected at 6, 8, and 12 hours after the morning dose on Day 14 (±2 days) unless 1 or more of these time points was waived in writing by the Sponsor ahead of time. The following windows were permitted for the collection of PK samples: ±2 minutes for samples collected ≤2 hours post-dose and ±5 minutes for samples collected >2 hours post-dose.
² On Days 3 (±1 day), 7 (±1 day), and 10 (±1 day), all subjects reported to the clinic for an outpatient visit. Blood samples for PK analysis were taken prior to the morning dose of study drug (within 5 minutes). Subjects in the intensive PK subgroup reported to the clinic on the day following their last dose of study drug (Day 15) and on Day 16 (+1 day) and a PK sample was collected at each visit. Subjects in the sparse PK subgroup may have reported to the clinic on either Day 15 or Day 16 (+1 day) and a PK sample was collected at this visit.
³ Written informed consent for the study was obtained from all subjects before any study procedures were performed.
⁴ Screening procedures, including vital sign assessments, may have been repeated no more than 2 times for eligibility purposes.
⁵ Any medications administered 28 days prior to the first dose of study drug and during the study period must have been recorded. Starting on Day 1, concomitant medications were recorded by the subject prior to each dose and recorded at each outpatient visit prior to each dose.
⁶ Aes were monitored and documented at each outpatient visit from the time first dose of study drug until study participation was complete.
⁷ A complete physical examination consisted of general appearance, skin, head, eyes, ears, mouth, oropharynx, neck, heart, lungs, abdomen, extremities, and neuromuscular system. A limited physical examination consisted of a minimum of general appearance, skin, heart, lungs, and abdomen.
⁸ Height was measured at the Screening Visit only. BMI was calculated at the Screening Visit using height and weight collected at the Screening Visit. At the time of randomization (Day 1), BMI was calculated using weight from the time of randomization (Day 1) and height from the Screening Visit.
⁹ Vital signs included heart rate, blood pressure, respiration rate, and temperature and were collected while the subject was sitting after a minimum 5-minute rest.
¹⁰ FSH was only measured for females who had been postmenopausal for at least 1 year and were not surgically sterile.
¹¹At the time of randomization (Day 1), a POC drug test was performed for use in assessing eligibility. A urine sample was also sent to the central lab for confirmatory testing.
¹² A pregnancy test was only performed in females who were not surgically sterile (or postmenopausal for at least 1 year [with confirmed FSH in postmenopausal range at the Screening Visit]). A serum pregnancy test was performed by the central laboratory on Days −28 to −11 and Day 14 (±2 days). A POC pregnancy test was initially performed on the Day −3 to −1 visit for use in assessing randomization eligibility.
¹³ All 12-lead ECGs were performed in triplicate approximately 1 minute apart after the subject had been resting in the supine position for at least 10 minutes. Twelve-lead ECGs were printed and interpreted as soon as possible. A digital recording of all ECGs from randomized subjects was submitted to a central reviewer. ECGs should have been completed ±15 minutes from all specified time points.
¹⁴On Days 1 and 14 (±2 days), ECGs were performed prior to the morning dose and at 2, 4, 6, and 12 hours (as applicable) after the morning dose and should have been completed ±15 minutes from the specified time points. For subjects participating in the intensive PK, additional ECGs were performed at 4, 6, and 12 hours (if applicable) after the morning dose). If collection of specific individual PK samples (including, but not limited to, the samples at 8 and/or 12 hours post-dose for the intense PK subgroup) were not required based on written Sponsor approval, the final ECGs for these visits must have been collected when the final PK sample was obtained.
¹⁵ Subjects had baseline GEBT performed between Days −10 and −3 after a minimum 14-day washout from motility agents and on Day 14 (±2 days) 30 minutes after study drug administration. Subjects fasted for a minimum of 8 hours before the breath test. Breath samples were obtained twice prior to consumption of a standardized, ¹³C-enriched meal and then on Days −10 to −3 at 45, 90, 120, 150, 180, and 240 minutes post meal after an 8-hour fast and on Day 14 (±2 days) at 45, 90, 120, 150, 180, and 240 minutes starting 30 minutes after study drug administration. Fasting glucose was assessed before GEBT to ensure a blood glucose level of ≤275 mg/dL.
¹⁶ Subjects were instructed to complete the ANMS GCSI-DD questionnaire daily at least 14 days prior to randomization (Day 1). Materials were dispensed at the Screening Visit. Adherence to ANMS GCSI-DD was assessed at the time of randomization (Day 1) and must have been ≥75% to be eligible for the study. Questionnaires should have been completed in the evening at approximately the same time each day. The PAGI-SYM questionnaire was completed at the study site on Days −10 to −3 prior to the start of the GEBT and on Day 14 (±2 days) prior to the final dose of study drug. The Clinical Grading Assessment Scales were completed on Day 14 (±2 days) prior to the start of the final dose of study drug and start of the GEBT.
¹⁷Sample collected pre dose. The Day 1 prolactin sample was not required to be repeated if a prolactin sample was obtained for the Pre-Randomization Visit in the last 72 hours.
¹⁸ Study drug was dispensed with treatment assigned on Day 1.
¹⁹ All subjects took a dose of blinded study drug (deudomperidone or placebo capsules) orally at 8:00 AM (±2 hours) and 8:00 PM (±2 hours) with approximately 240 mL of water. A 12-hour interval was maintained between each dose for a given individual. Fasting requirement for Day 1: Subjects participating in the intensive PK subgroup should have made an effort to fast for a minimum of 8 hours before the morning dose of study drug. However, a light meal may have been consumed >2 hours prior to the morning dose of study drug if determined that food should be consumed (i.e., due to low blood glucose levels or symptoms of hypoglycemia). Subjects should have made an effort to fast for 4 hours after the morning dose of study drug; however, if determined that food should be consumed, it was permissible to break the specified fast to ensure subject safety. For the evening dose, subjects fasted for >2 hours before and 1 hour after study drug. The evening dose was self-administered by the subject. Subjects participating in the sparse PK subgroup should have made an effort to fast for a minimum of 8 hours before the morning dose of study drug. However, a light meal may have been consumed >2 hours prior to the morning dose of study drug if determined that food should be consumed by the subject (i.e., due to low blood glucose levels or symptoms of hypoglycemia). Subjects should have fasted for >2 hours after the morning dose of study drug; however, if determined that food should be consumed, it was permissible to break the specified fast to ensure subject safety. For the evening dose, subjects fasted for a minimum of 2 hours before and 1 hour after the dose of study drug. The evening dose was self-administered by the subject. If a subject reported vomiting during the treatment period, subjects should have attempted to note the time of vomiting in relation to time of study drug administration and report this to site staff during scheduled clinic visits.
²⁰ Study drug accountability occurred on Days 3, 7, 10, and 14 or on Early Termination if applicable. Unused study drug was only collected on Day 14 (±2 days) or on Early Termination if applicable.
²¹ For subjects who provided written informed consent to participate in the optional PGx assessment, a blood sample was collected at any time during the subject's participation in the study.
²² Subjects participating in the intensive PK subgroup reported to the clinic on Day 15 and on Day 16 (+1 day) where a PK sample was collected at each respective visit. Subjects in the sparse PK subgroup may have reported to the clinic on either Day 15 or Day 16 (+1 day) and a PK sample was collected at the selected visit.
²³For subjects participating in the sparse PK subgroup and who were only required to complete either Day 15 or Day 16 (+1 day), these procedures were performed on Day 16 (+1 day) if Day 15 was not performed.

### (ii) Appropriateness of Measurements

The GEBT and ANMS GCSI-DD were used as efficacy measurements. The GEBT is an FDA-approved measurement for gastric emptying and is an appropriate endpoint for the 2-week treatment period used in this study. Use of the ANMS GCSI-DD is standard for this type of study and assesses the core signs and symptoms as endpoints as suggested in the FDA guidance. The PK and safety measurements in this study are widely used and recognized as reliable, accurate, and relevant.

(iii) Efficacy Variables

### Primary Efficacy Variable

The primary efficacy variable was the change from baseline in gastric emptying as measured by GEBT at Day 14.

### Secondary Efficacy Variables

The secondary efficacy variables were the following:
The change from baseline in gastric emptying as measured by GEBT T½ at Day 14;
The change from baseline in ANMS GCSI-DD total scores; and
The change from baseline in ANMS GCSI-DD subscale scores.

### (iv) American Neurogastroenterology and Motility Society Gastroparesis Cardinal Symptom Index Daily Diary:

### Protocol-Specified Scoring Method

The ANMS GCSI-DD covers 5 core relevant symptoms of gastroparesis: nausea, early satiety, postprandial fullness, upper abdominal pain, and vomiting. Bloating is included as an exploratory symptom. Symptoms are rated on a severity numeric response scale from 0 (none) to 4 (very severe). Vomiting is captured on a frequency response scale and scored as follows: 0 for no episodes, 1 for 1 episode, 2 for 2 episodes, 3 for 3 episodes, and 4 for 4 or more episodes. Subjects were asked to recall symptoms over the previous 24 hours and complete the ANMS GCSI-DD patient-reported symptom questionnaire for at least 14 days prior to randomization, on the day of randomization, and daily from Days 1 to 14 (±2 days).

### American Neurogastroenterology and Motility Society Manual Scoring Method

The severity scores of 4 gastroparesis-related symptoms (nausea, early satiety, postprandial fullness, upper abdominal pain) range from 0-none to 4-very severe. The vomiting score assesses the number of vomiting episodes during the day, capped at a maximum of 4; thus, the scores for vomiting range from 0 (no episodes of vomiting) to 4 (4 or more episodes of vomiting). Vomiting frequency is scored as 0 episodes, 1 episode, 2 episodes, 3 episodes, or 4 or more episodes (capped as 4).

### (vi) Exploratory Efficacy Variables

The exploratory efficacy variables were the following:
The change from baseline in gastroparesis symptoms as measured by PAGI-SYM questionnaire scores; and
The Clinical Grading Assessment Scale at Day 14.

### Patient Assessment of Gastrointestinal Disorders Symptom Severity Index:

The PAGI-SYM includes 6 subscales: heartburn/regurgitation, fullness/early satiety, nausea/vomiting, bloating, upper abdominal pain, and lower abdominal pain.

### Clinical Grading Assessment Scale:

For assessment of global clinical response, the following question was utilized at Day 14: “In thinking about the last 2 weeks, how would you say your stomach/gastroparesis-related problems/symptoms have been compared to the period before you started treatment on this study?” Responses were the following: improved, no change, or worsened. Subjects were asked to quantify their therapeutic response using the Clinical Patient Grading Assessment Scale. Subjects picked a number over a range (+7=completely better; 0=no change; −7=very much worse) that best answered the question.

(vii) Drug Concentrations and Other Pharmacokinetic Variables

The PK variables were concentrations and derived PK parameters of deuterated domperidone following multiple doses of deudomperidone. The following PK parameters were calculated for deuterated domperidone from plasma concentrations (Day 1) using a noncompartmental method, if the data permitted:
Cₘₐₓ determined directly from the concentration-time profile. If the maximum value occurred at more than 1 time point, Cₘₐₓ was defined as the first maximum value;
Tₘₐₓ; if the maximum value occurred at more than 1 time point, Tₘₐₓ was defined as the first time point with this value; and
AUC₀₋₁₂.

The following PK parameters were calculated for deuterated domperidone using measured plasma concentrations following the final dose of deudomperidone (AM of Day 14), if the data permitted:
Cₘₐₓ determined directly from the concentration-time profile. If the maximum value occurred at more than 1 time point, Cₘₐₓ was defined as the first maximum value;
Tₘₐₓ; if the maximum value occurred at more than 1 time point, Tₘₐₓ was defined as the first time point with this value;
AUCₜₐᵤ calculated using the trapezoidal method;
AUC₀₋ₜ;

$${AUC}_{0 - ∞} ; calculated as ( {AUC}_{0 - t} + {C}_{last} / λ z ) ;$$

$${AUC}_{\% extrap} ; represented as ( 1 - {AUC}_{0 - t} / {AUC}_{0 - ∞} ) \times 100 ;$$

λz calculated from a semi-logarithmic plot of the plasma concentration versus time curve. The parameter was calculated by linear least-squares regression analysis using points in the terminal logarithmic-linear phase;
T½ calculated as natural logarithm (2)/λz;
CL/F calculated as Dose/AUC_{0-∞};
Vz/F calculated as Dose/(λz×AUC_{0-∞});
R_{cmax} calculated as Cₘₐₓ on Day 14/Cₘₐₓ on Day 1; and
R_{AUC} calculated as AUCₜₐᵤ on Day 14/AUCₜₐᵤ on Day 1.
   (viii) Safety Variables

Safety variables included Aes, clinical laboratory results (including prolactin), vital signs, ECGs, and physical examination findings.

### F. Study Subjects

### (i) Disposition of Subjects

Based on emerging information from the current study as well as other ongoing studies, the planned dose levels and cohort sizes for the study were modified as follows:
Cohort 1 was expanded to a total of 41 randomized subjects, and a 5 mg BID cohort (Cohort 3) was planned;
The dose level of Cohort 2 was changed to 20 mg QD, administered as a morning dose of a deudomperidone 20 mg capsule and an evening dose of a matching placebo capsule in order to maintain the study blind such that each dose may have involved active study drug or placebo; and
Cohort 2 expansion to 18 subjects was planned at a dose level of 15 mg BID consisting of a 10 mg and a 5 mg capsule taken as a single dose. Due to changes in manufacturing methods of the 10 mg capsules, the Sponsor opted not to move forward with the 15 mg BID dose level to avoid introducing potential variability between lots and thus confounding data analysis. The 5 mg BID (Cohort 3) was expanded to a total of 18 subjects, but only 12 subjects were randomized.

Table 5 summarizes subject disposition for the ITT Population.

A total of 72 subjects were enrolled in 1 of 3 cohorts. Fifty-four subjects were randomized into 1 of the following deudomperidone treatment groups:
10 mg BID deudomperidone (30 subjects randomized);
20 mg QD deudomperidone (15 subjects randomized); or
5 mg BID deudomperidone (9 subjects randomized).

Eighteen subjects were randomized to receive placebo.

Of the randomized subjects, 29 (96.7) subjects in the 10 mg BID deudomperidone treatment group, 15 (100.0%) subjects in the 20 mg QD deudomperidone treatment group, 9 (100.0%) subjects in the 5 mg BID deudomperidone treatment group, and 18 (100.0%) subjects in the pooled placebo treatment group completed the study. There was 1 (1.4%) subject who withdrew early from the study. This subject (Subject 113-010) received 10 mg BID deudomperidone and was withdrawn.

TABLE 5
Subject Disposition - Intent-to-Treat Population

| | Deudomperidone | | | | | | Pooled | | | |
|---|---|---|---|---|---|---|---|---|---|---|
| | 10 mg BID | | 20 mg QD | | 5 mg BID | | Placebo | | Total | |
| | (N = 30) | | (N = 15) | | (N = 9) | | (N = 18) | | (N = 72) | |
| | n (%) | | n (%) | | n (%) | | n (%) | | n (%) | |
| Subjects who were | 30 | (100.0) | 15 | (100.0) | 9 | (100.0) | 18 | (100.0) | 72 | (100.0) |
| randomized | | | | | | | | | | |
| Intensive PK schedule | 23 | (76.7) | 12 | (80.0) | 7 | (77.8) | 14 | (77.8) | 56 | (77.8) |
| Sparse PK schedule | 7 | (23.3) | 3 | (20.0) | 2 | (22.2) | 4 | (22.2) | 16 | (22.2) |
| Subjects who were | 30 | (100.0) | 15 | (100.0) | 9 | (100.0) | 18 | (100.0) | 72 | (100.0) |
| randomized and received | | | | | | | | | | |
| any amount of study | | | | | | | | | | |
| drug | | | | | | | | | | |
| Subjects who completed | 29 | (96.7) | 15 | (100.0) | 9 | (100.0) | 18 | (100.0) | 71 | (98.6) |
| the study | | | | | | | | | | |
| Subjects who withdrew | 1 | (3.3) | 0 | (0.0) | 0 | (0.0) | 0 | (0.0) | 1 | (1.4) |
| early from the study | | | | | | | | | | |

Primary reason for discontinuation

| | | | | | | | | | | |
|---|---|---|---|---|---|---|---|---|---|---|
| Other | 1 | (3.3) | 0 | (0.0) | 0 | (0.0) | 0 | (0.0) | 1 | (1.4) |
| Subjects who | 0 | (0.0) | 0 | (0.0) | 0 | (0.0) | 0 | (0.0) | 0 | (0.0) |
| prematurely withdrew | | | | | | | | | | |
| from the study due to the | | | | | | | | | | |
| COVID-19 pandemic | | | | | | | | | | |

% = 100 × n/N.
n = number of subjects meeting specified criteria;
N = number of subjects in the treatment group

### (ii) Data Sets Analyzed

Table 6 describes the analysis populations for the study.
The ITT Population included 72 subjects (30 subjects in the 10 mg BID deudomperidone treatment group, 15 subjects in the 20 mg QD deudomperidone treatment group, 9 subjects in the 5 mg BID deudomperidone treatment group, and 18 subjects in the pooled placebo treatment group), and was identical to the MITT Population, the PD Evaluable Population, and the Safety Population.
The PP Population included 69 subjects (29 subjects in the 10 mg BID deudomperidone treatment group, 14 subjects in the 20 mg QD deudomperidone treatment group, 8 subjects in the 5 mg BID deudomperidone treatment group, and 18 subjects in the pooled placebo treatment group).

The PK Population included 54 subjects (30 subjects in the 10 mg BID deudomperidone treatment group, 15 subjects in the 20 mg QD deudomperidone treatment group, and 9 subjects in the 5 mg BID deudomperidone treatment group) and was identical to the PK Evaluable Population.

TABLE 6
Analysis Populations - Intent-to-Treat Population

| | Deudomperidone | | | | | |
|---|---|---|---|---|---|---|
| | 10 mg | 20 mg | 5 mg | Pooled | | |
| | BID | QD | BID | Placebo | Total | |
| | (N = 30) | (N = 15) | (N = 9) | (N = 18) | (N = 72) | |
| | n (%) | n (%) | n (%) | n (%) | n (%) | |
| ITT Population ¹ | 30 (100.0) | 15 (100.0) | 9 (100.0) | 18 (100.0) | 72 | (100.0) |
| MITT Population ² | 30 (100.0) | 15 (100.0) | 9 (100.0) | 18 (100.0) | 72 | (100.0) |
| PP Population ³ | 29 (96.7) | 14 (93.3) | 8 (88.9) | 18 (100.0) | 69 | (95.8) |
| PK Population ⁴ | 30 (100.0) | 15 (100.0) | 9 (100.0) | — | 54 | (75.0) |
| PK Evaluable Population ⁵ | 30 (100.0) | 15 (100.0) | 9 (100.0) | — | 54 | (75.0) |
| PD Evaluable Population ⁶ | 30 (100.0) | 15 (100.0) | 9 (100.0) | 18 (100.0) | 72 | (100.0) |
| Safety Population ⁷ | 30 (100.0) | 15 (100.0) | 9 (100.0) | 18 (100.0) | 72 | (100.0) |

% = 100 × n/N.
¹ The ITT Population included all randomized subjects.
² The MITT Population included all randomized subjects who received at least 1 dose of study drug.
³ The PP Population included all subjects in the MITT Population without major protocol violations and who did not meet any of the criteria as specified in the Statistical Analysis Plan.
⁴ The PK Population included all subjects who received at least 1 dose of deudomperidone and had at least 1 quantifiable post-dose plasma deudomperidone concentration.
⁵ The PK Evaluable Population included subjects who received deudomperidone and had sufficient plasma concentration data to characterize at least 1 PK parameter.
⁶ The PD Evaluable Population included all subjects who received study drug and had at least 1 prolactin value following the start of study drug administration.
⁷ The Safety Population consisted of all randomized subjects who received any study drug. The data from subjects who received placebo in all cohorts were pooled for safety analyses.

(iii) Demographic and Other Baseline Characteristics

Table 7 summarizes demographic and baseline characteristics for the ITT Population. The mean age of subjects ranged from approximately 50.7 to 58.9 years, and mean BMI ranged from approximately 28.89 to 32.00 kg/in². The majority of subjects were female. In total, 83.3% of subjects were White, and 15.3% of subjects were Black or African American. The majority of subjects were Hispanic or Latino. For all treatment groups, the majority of subjects (51 [70.8%] subjects) had diabetic gastroparesis etiology. Among subjects with diabetic gastroparesis, the numbers of subjects with and without neuropathy were similar except for Cohort 2, where the large majority of subjects had neuropathy. More subjects had a baseline GEBT ≥110 kPCD than <100 kPCD. These observed differences in demographic and baseline characteristics are considered unlikely to have influenced the data interpretation and conclusions.

TABLE 7
Demographic and Baseline Characteristics - Intent-to-Treat Population

| | Deudomperidone | | | Pooled | |
|---|---|---|---|---|---|
| | 10 mg BID | 20 mg QD | 5 mg BID | Placebo | Total |
| Characteristic | (N = 30) | (N = 15) | (N = 9) | (N = 18) | (N = 72) |
| Statistic/Category | n (%) | n (%) | n (%) | n (%) | n (%) |

Age at screening (years)

| n | 30 | | 15 | | 9 | | 18 | | 72 | |
|---|---|---|---|---|---|---|---|---|---|---|
| Mean (SD) | 58.9 | (7.44) | 58.1 | (7.00) | 50.7 | (11.91) | 56.1 | (11.23) | 57.0 | (9.25) |

Sex, n (%)

| | | | | | | | | | | |
|---|---|---|---|---|---|---|---|---|---|---|
| Male | 4 | (13.3) | 4 | (26.7) | 1 | (11.1) | 5 | (27.8) | 14 | (19.4) |
| Female | 26 | (86.7) | 11 | (73.3) | 8 | (88.9) | 13 | (72.2) | 58 | (80.6) |

Ethnicity, n (%)

| | | | | | | | | | | |
|---|---|---|---|---|---|---|---|---|---|---|
| Hispanic or Latino | 19 | (63.3) | 9 | (60.0) | 3 | (33.3) | 14 | (77.8) | 45 | (62.5) |
| Not Hispanic or Latino | 11 | (36.7) | 5 | (33.3) | 6 | (66.7) | 4 | (22.2) | 26 | (36.1) |
| Not reported | 0 | (0.0) | 1 | (6.7) | 0 | (0.0) | 0 | (0.0) | 1 | (1.4) |

Race, n (%)

| | | | | | | | | | | |
|---|---|---|---|---|---|---|---|---|---|---|
| White | 24 | (80.0) | 12 | (80.0) | 7 | (77.8) | 17 | (94.4) | 60 | (83.3) |
| Black or African | 6 | (20.0) | 3 | (20.0) | 2 | (22.2) | 0 | (0.0) | 11 | (15.3) |
| American | | | | | | | | | | |
| Other | 0 | (0.0) | 0 | (0.0) | 0 | (0.0) | 1 | (5.6) | 1 | (1.4) |

Gastroparesis type, n (%)

| | | | | | | | | | | |
|---|---|---|---|---|---|---|---|---|---|---|
| Idiopathic | 7 | (23.3) | 5 | (33.3) | 2 | (22.2) | 7 | (38.9) | 21 | (29.2) |
| Diabetic | 23 | (76.7) | 10 | (66.7) | 7 | (77.8) | 11 | (61.1) | 51 | (70.8) |
| With neuropathy ¹ | 13 | (56.5) | 8 | (80.0) | 3 | (42.9) | 6 | (54.5) | 30 | (58.8) |
| Without neuropathy ¹ | 10 | (43.5) | 2 | (20.0) | 4 | (57.1) | 5 | (45.5) | 21 | (41.2) |

Baseline GEBT T½, n (%)

| | | | | | | | | | | |
|---|---|---|---|---|---|---|---|---|---|---|
| <110 kPCD | 11 | (36.7) | 5 | (33.3) | 0 | (0.0) | 7 | (38.9) | 23 | (31.9) |
| ≥110 kPCD | 19 | (63.3) | 6 | (40.0) | 2 | (22.2) | 7 | (38.9) | 34 | (47.2) |

Height at screening (cm)

| n | 30 | | 15 | | 9 | | 18 | | 72 | |
|---|---|---|---|---|---|---|---|---|---|---|
| Mean (SD) | 165.53 | (9.966) | 163.85 | (8.210) | 163.40 | (9.191) | 163.84 | (8.614) | 164.49 | (9.052) |

Weight at screening (kg)

| n | 30 | | 15 | | 9 | | 18 | | 72 | |
|---|---|---|---|---|---|---|---|---|---|---|
| Mean (SD) | 83.23 | (18.268) | 79.03 | (17.455) | 77.26 | (16.876) | 86.21 | (17.749) | 82.35 | (17.710) |

BMI at screening (kg/m²)

| n | 30 | | 15 | | 9 | | 18 | | 72 | |
|---|---|---|---|---|---|---|---|---|---|---|
| Mean (SD) | 30.16 | (4.704) | 29.43 | (6.189) | 28.89 | (5.859) | 32.00 | (5.631) | 30.31 | (5.404) |

% = 100 × n/N.
Baseline was defined as the last measurement prior to the first dose of study drug.
¹ Percentage was calculated based off diabetic subjects.

### (vi) Prior and Concomitant Medications

Overall, 70 (97.2%) subjects took a protocol-permitted concomitant medication and 70 (97.2%) subjects took any prior medication.

In this study, 20 (27.8) subjects overall took a protocol-permitted rescue medication as reported by subjects in their subject diary.

No medication use had the potential to confound results.

### G. Efficacy, Pharmacodynamic, and Pharmacokinetic Results

### (i) Efficacy Results and Tabulations of Individual Subject Data

### Gastric Emptying Time

Table 8 presents gastric emptying time at baseline and Day 14 and the change and percent change in gastric emptying time from baseline to Day 14 as measured by GEBT T½ for the ITT Population. For the 10 mg BID deudomperidone group, gastric emptying time was reduced from baseline for deudomperidone and placebo treatment groups, and the change and percent change from baseline (gathered on Days −10 to −3) in gastric emptying as measured by GEBT T_{1/2} to Day 14 (±2 days) was greater compared to placebo, suggesting faster gastric emptying after deudomperidone administration.

TABLE 8
Gastric Emptying Time - Intent-to-Treat Population

| GEBT Parameter | Cohort 1 | | |
|---|---|---|---|
| Visit | | Deudomperidone | |
| Statistic | | 10 mg BID (N = 30) | Placebo (N = 11) |

T½ (kPCD)
Baseline

| | | |
|---|---|---|
| n | 30 | 11 |

| | | | | |
|---|---|---|---|---|
| Mean (SD) | 132.97 | (36.228) | 119.50 | (34.654) |

Day 14

| | | |
|---|---|---|
| n | 29 | 11 |

| | | | | |
|---|---|---|---|---|
| Mean (SD) | 123.16 | (30.881) | 111.45 | (20.923) |

Change from baseline to Day 14

| | | |
|---|---|---|
| n | 29 | 11 |

| | | | | |
|---|---|---|---|---|
| Mean (SD) | −10.60 | (37.240) | −8.05 | (35.640) |

Percent change from baseline to Day 14

| | | |
|---|---|---|
| n | 29 | 11 |

| | | | | |
|---|---|---|---|---|
| Mean (SD) | −4.23 | (24.278) | −1.35 | (26.533) |

Baseline was defined as the last GEBT begun prior to the first dose of study drug.
GEBT results were reported using kPCD, a mathematical expression of a test subject's ¹³CO₂ excretion rate per minute at any measurement time t relative to the dose of ¹³C contained in the test meal.
kPCD(t) = 1000 × [percent ¹³C dose excreted as ¹³CO₂ per minute (min⁻¹) at time t].

Table 9 presents gastric emptying time at baseline and Day 14 and the change in gastric emptying time from baseline to Day 14 as measured by GEBT by time point for the ITT Population. All pre-meal consumption results were 0 and were therefore excluded from the analysis.

TABLE 9
Gastric Emptying Time by Time Point - Intent-to-Treat Population

| GEBT Parameter | Cohort 1 | | |
|---|---|---|---|
| Visit | | Deudomperidone | |
| Statistic | | 10 mg BID (N = 30) | Placebo (N = 11) |

GEBT 45 minutes post meal consumption
Baseline

| | | |
|---|---|---|
| n | 30 | 11 |

| Mean (SD) | 8.88 | (4.113) | 9.56 | (6.040) |
|---|---|---|---|---|
| Day 14 | | | | |
| n | 29 | | 11 | |
| Mean (SD) | 10.74 | (3.038) | 10.70 | (3.266) |

Change from baseline to Day 14

| n | 29 | | 11 | |
|---|---|---|---|---|
| Mean (SD) | 1.96 | (4.091) | 1.14 | (6.210) |

Percent change from baseline to Day 14

| n | 29 | | 11 | |
|---|---|---|---|---|
| Mean (SD) | 89.31 | (266.803) | −679.95 | (2500.333) |

GEBT 90 minutes post meal consumption
Baseline

| n | 30 | | 11 | |
|---|---|---|---|---|
| Mean (SD) | 20.53 | (9.016) | 23.55 | (10.522) |

Day 14

| n | 29 | | 11 | |
|---|---|---|---|---|
| Mean (SD) | 23.60 | (6.128) | 25.10 | (6.551) |

Change from baseline to Day 14

| n | 29 | | 11 | |
|---|---|---|---|---|
| Mean (SD) | 3.37 | (9.287) | 1.55 | (10.056) |

Percent change from baseline to Day 14

| n | 29 | | 11 | |
|---|---|---|---|---|
| Mean (SD) | 56.73 | (125.218) | 43.43 | (117.796) |

GEBT 120 minutes post meal consumption
Baseline

| n | 30 | | 11 | |
|---|---|---|---|---|
| Mean (SD) | 29.02 | (11.549) | 31.45 | (12.536) |

Day 14

| n | 29 | | 11 | |
|---|---|---|---|---|
| Mean (SD) | 32.68 | (8.901) | 33.61 | (9.146) |

Change from baseline to Day 14

| n | 29 | | 11 | |
|---|---|---|---|---|
| Mean (SD) | 3.86 | (11.008) | 2.16 | (11.547) |

Percent change from baseline to Day 14

| n | 29 | | 11 | |
|---|---|---|---|---|
| Mean (SD) | 40.17 | (102.994) | 29.78 | (83.476) |

GEBT 150 minutes post meal consumption
Baseline

| n | 30 | | 11 | |
|---|---|---|---|---|
| Mean (SD) | 35.97 | (12.946) | 36.89 | (11.820) |

Day 14

| n | 29 | | 11 | |
|---|---|---|---|---|
| Mean (SD) | 37.13 | (10.002) | 39.23 | (8.021) |

Change from baseline to Day 14

| n | 29 | | 11 | |
|---|---|---|---|---|
| Mean (SD) | 1.25 | (12.261) | 2.34 | (10.324) |

Percent change from baseline to Day 14

| n | 29 | | 11 | |
|---|---|---|---|---|
| Mean (SD) | 23.46 | (82.609) | 21.57 | (64.614) |

GEBT 180 minutes post meal consumption
Baseline

| n | 30 | | 11 | |
|---|---|---|---|---|
| Mean (SD) | 39.02 | (13.258) | 40.94 | (12.526) |
| Day 14 | | | | |
| n | 29 | | 11 | |
| Mean (SD) | 40.96 | (11.080) | 43.67 | (8.078) |

Change from baseline to Day 14

| n | 29 | | 11 | |
|---|---|---|---|---|
| Mean (SD) | 1.85 | (12.445) | 2.74 | (10.959) |

Percent change from baseline to Day 14

| n | 29 | | 11 | |
|---|---|---|---|---|
| Mean (SD) | 20.31 | (65.491) | 17.80 | (46.488) |

GEBT 240 minutes post meal consumption

| Baseline | | | | |
|---|---|---|---|---|
| n | 30 | | 11 | |
| Mean (SD) | 41.89 | (12.750) | 41.85 | (8.953) |
| Day 14 | | | | |
| n | 29 | | 11 | |
| Mean (SD) | 41.41 | (10.422) | 44.57 | (7.928) |

Change from baseline to Day 14

| n | 29 | | 11 | |
|---|---|---|---|---|
| Mean (SD) | −0.79 | (11.821) | 2.73 | (8.144) |

Percent change from baseline to Day 14

| n | 29 | | 11 | |
|---|---|---|---|---|
| Mean (SD) | 9.46 | (58.567) | 9.87 | (25.864) |

All pre-consumption results were 0 and were therefore excluded from the analysis.
Baseline was defined as the last GEBT begun prior to the first dose of study drug.
GEBT results were reported using kPCD, a mathematical expression of a test subject's ¹³CO₂ excretion rate per minute at any measurement time t relative to the dose of ¹³C contained in the test meal.
kPCD(t) = 1000 × [percent ¹³C dose excreted as ¹³CO₂ per minute (min⁻¹) at time t].

### American Neurogastroenterology and Motility Society Gastroparesis Cardinal Symptom Index Daily Diary

Table 10 presents the ANMS GCS-DD total and subscale scores and change and percent change from baseline for the ITT Population using the protocol-specified scoring method. For all deudomperidone treatment groups, ANMS GCSI-DD total score and all subscale scores decreased from baseline at Day 14/End of Treatment. Subjects receiving placebo also experienced decreases in ANMS GCSI-DD total and subscale scores.

TABLE 10
ANMS GCSI-DD Total and Subscale Scores (Protocol-Specified
Scoring Method) - Intent-to-Treat Population

| | Cohort 1 | | Cohort 2 | | Cohort 3 | |
|---|---|---|---|---|---|---|
| | Deudomperi- | | Deudomperi- | | Deudomperi- | |
| Parameter | done | | done | | done | |
| Visit | 10 mg BID | Placebo | 20 mg QD | Placebo | 5 mg BID | Placebo |
| Statistic | (N = 30) | (N = 11) | (N = 15) | (N = 4) | (N = 9) | (N = 3) |

ANMS GCSI-DD total score
Baseline

| | | | | | | |
|---|---|---|---|---|---|---|
| n | 30 | 11 | 15 | 4 | 9 | 3 |
| Mean (SD) | 2.42 | 2.40 | 2.40 | 2.33 | 2.33 | 2.81 |
| | (0.666) | (0.464) | (0.530) | (0.276) | (0.583) | (0.594) |

Day 14/EOT

| | | | | | | |
|---|---|---|---|---|---|---|
| n | 29 | 11 | 15 | 4 | 9 | 3 |
| Mean (SD) | 1.40 | 1.23 | 1.08 | 0.83 | 1.78 | 1.33 |
| | (0.731) | (0.870) | (0.866) | (0.694) | (1.051) | (0.882) |

Change from baseline to Day 14/EOT

| | | | | | | |
|---|---|---|---|---|---|---|
| n | 29 | 11 | 15 | 4 | 9 | 3 |
| Mean (SD) | −1.04 | −1.18 | −1.33 | −1.50 | −0.56 | −1.48 |
| | (0.612) | (0.754) | (0.864) | (0.803) | (0.665) | (1.233) |

Percent change from baseline to Day 14/EOT

| | | | | | | |
|---|---|---|---|---|---|---|
| n | 29 | 11 | 15 | 4 | 9 | 3 |
| Mean (SD) | −43.64 | −49.45 | −55.24 | −63.25 | −26.84 | −48.79 |
| | (24.813) | (33.736) | (35.011) | (34.016) | (31.291) | (40.507) |

ANMS GCSI-DD nausea/vomiting subscale score
Baseline

| | | | | | | |
|---|---|---|---|---|---|---|
| n | 30 | 11 | 15 | 4 | 9 | 3 |
| Mean (SD) | 1.62 | 1.55 | 1.60 | 1.79 | 1.52 | 2.00 |
| | (0.752) | (0.757) | (0.623) | (0.798) | (0.556) | (0.500) |

Day 14/EOT

| | | | | | | |
|---|---|---|---|---|---|---|
| n | 29 | 11 | 15 | 4 | 9 | 3 |
| Mean (SD) | 0.74 | 0.59 | 0.40 | 0.38 | 1.00 | 0.50 |
| | (0.561) | (1.020) | (0.431) | (0.479) | (1.061) | (0.500) |

Change from baseline to Day 14/EOT

| | | | | | | |
|---|---|---|---|---|---|---|
| n | 29 | 11 | 15 | 4 | 9 | 3 |
| Mean (SD) | −0.89 | −0.95 | −1.20 | −1.42 | −0.52 | −1.50 |
| | (0.820) | (0.583) | (0.812) | (0.776) | (0.719) | (0.866) |

Percent change from baseline to Day 14/EOT

| | | | | | | |
|---|---|---|---|---|---|---|
| n | 29 | 11 | 15 | 4 | 9 | 3 |
| Mean (SD) | −52.41 | −69.49 | −67.94 | −81.36 | −41.08 | −72.22 |
| | (37.374) | (38.394) | (34.799) | (25.729) | (47.221) | (25.459) |

ANMS GCSI-DD postprandial fullness/early satiety subscale score
Baseline

| | | | | | | |
|---|---|---|---|---|---|---|
| n | 30 | 11 | 15 | 4 | 9 | 3 |
| Mean (SD) | 2.84 | 2.85 | 2.74 | 2.63 | 2.78 | 3.33 |
| | (0.674) | (0.575) | (0.630) | (0.250) | (0.791) | (0.577) |

Day 14/EOT

| | | | | | | |
|---|---|---|---|---|---|---|
| n | 29 | 11 | 15 | 4 | 9 | 3 |
| Mean (SD) | 1.71 | 1.64 | 1.37 | 1.13 | 2.00 | 1.83 |
| | (0.978) | (1.027) | (1.008) | (0.854) | (1.146) | (0.764) |

Change from baseline to Day 14/EOT

| | | | | | | |
|---|---|---|---|---|---|---|
| n | 29 | 11 | 15 | 4 | 9 | 3 |
| Mean (SD) | −1.16 | −1.21 | −1.38 | −1.50 | −0.78 | −1.50 |
| | (0.771) | (0.961) | (1.011) | (1.080) | (0.816) | (0.866) |

Percent change from baseline to Day 14/EOT

| | | | | | | |
|---|---|---|---|---|---|---|
| n | 29 | 11 | 15 | 4 | 9 | 3 |
| Mean (SD) | −41.38 | −42.32 | −50.14 | −54.75 | −29.47 | −44.44 |
| | (29.096) | (36.042) | (36.594) | (36.522) | (32.944) | (25.459) |

ANMS GCSI-DD bloating subscale score
Baseline

| | | | | | | |
|---|---|---|---|---|---|---|
| n | 30 | 11 | 15 | 4 | 9 | 3 |
| Mean (SD) | 2.80 | 2.82 | 2.87 | 2.58 | 2.70 | 3.11 |
| | (0.824) | (0.480) | (0.710) | (0.500) | (0.696) | (1.018) |

Day 14/EOT

| | | | | | | |
|---|---|---|---|---|---|---|
| n | 29 | 11 | 15 | 4 | 9 | 3 |
| Mean (SD) | 1.76 | 1.45 | 1.47 | 1.00 | 2.33 | 1.67 |
| | (1.123) | (0.934) | (1.356) | (0.816) | (1.118) | (1.528) |

Change from baseline to Day 14/EOT

| | | | | | | |
|---|---|---|---|---|---|---|
| n | 29 | 11 | 15 | 4 | 9 | 3 |
| Mean (SD) | −1.08 | −1.36 | −1.40 | −1.58 | −0.37 | −1.44 |
| | (0.785) | (0.994) | (1.121) | (1.258) | (0.655) | (2.219) |

Percent change from baseline to Day 14/EOT

| | | | | | | |
|---|---|---|---|---|---|---|
| n | 29 | 11 | 15 | 4 | 9 | 3 |
| Mean (SD) | −41.78 | −46.73 | −51.82 | −55.95 | −16.16 | −33.33 |
| | (32.337) | (35.270) | (41.422) | (41.582) | (26.339) | (76.376) |

Baseline was defined as the mean score for the 3 days preceding randomization.
Day 14/EOT was defined as the last available score on or before the last date of treatment.
The ANMS GCSI-DD total score was the mean of the 3 subscale scores for severity of nausea/vomiting, postprandial fullness/early satiety, and bloating.

Table 11 presents the ANMS GCSI-DD total and subscale scores and change and percent change from baseline for the ITT Population using the ANMS GC SI-DD Manual scoring method. For all deudomperidone treatment groups, ANMS GCSI-DD total score and all subscale scores decreased from baseline at Day 14/EOT. Subjects receiving placebo also experienced decreases in ANMS GCSI-DD total and subscale scores.

TABLE 11
ANMS GCSI-DD Total and Subscale Scores (ANMS GCSI-DD
Manual Scoring Method) - Intent-to-Treat Population

| | | | Cohort 2 | | | |
|---|---|---|---|---|---|---|
| | Cohort 1 | | Deudomperi- | | Cohort 3 | |
| Parameter | Deudomperi- | | done | | Deudomperi- | |
| Visit | done 10 mg | Placebo | 20 mg QD | Placebo | done 5 mg | Placebo |
| Statistic | BID (N = 30) | (N = 11) | (N = 15) | (N = 4) | BID (N = 9) | (N = 3) |

ANMS GCSI-DD total score
Baseline

| | | | | | | |
|---|---|---|---|---|---|---|
| n | 30 | 11 | 15 | 4 | 9 | 3 |
| Mean | 2.14 | 2.15 | 2.13 | 2.18 | 2.16 | 2.56 |
| (SD) | (0.654) | (0.414) | (0.495) | (0.450) | (0.567) | (0.454) |

Day 14/EOT

| | | | | | | |
|---|---|---|---|---|---|---|
| n | 29 | 11 | 15 | 4 | 9 | 3 |
| Mean | 1.17 | 1.04 | 0.88 | 0.80 | 1.47 | 1.20 |
| (SD) | (0.582) | (0.833) | (0.671) | (0.730) | (1.082) | (0.693) |

Change from baseline to Day 14/EOT

| | | | | | | |
|---|---|---|---|---|---|---|
| n | 29 | 11 | 15 | 4 | 9 | 3 |
| Mean | −0.98 | −1.11 | −1.25 | −1.38 | −0.69 | −1.36 |
| (SD) | (0.684) | (0.699) | (0.831) | (0.672) | (0.736) | (0.707) |

Percent change from baseline to Day 14/EOT

| | | | | | | |
|---|---|---|---|---|---|---|
| n | 29 | 11 | 15 | 4 | 9 | 3 |
| Mean | −44.07 | −52.61 | −55.99 | −65.05 | −35.01 | −52.81 |
| (SD) | (26.803) | (33.887) | (35.840) | (34.164) | (36.277) | (28.361) |

ANMS GCSI-DD nausea subscale score
Baseline

| | | | | | | |
|---|---|---|---|---|---|---|
| n | 30 | 11 | 15 | 4 | 9 | 3 |
| Mean | 2.52 | 2.45 | 2.67 | 2.33 | 2.59 | 3.00 |
| (SD) | (0.786) | (0.563) | (0.854) | (0.903) | (0.778) | (0.000) |

Day 14/EOT

| | | | | | | |
|---|---|---|---|---|---|---|
| n | 29 | 11 | 15 | 4 | 9 | 3 |
| Mean | 1.34 | 0.82 | 0.73 | 0.75 | 1.67 | 1.00 |
| (SD) | (1.010) | (0.982) | (0.799) | (0.957) | (1.323) | (1.000) |

Change from baseline to Day 14/EOT

| | | | | | | |
|---|---|---|---|---|---|---|
| n | 29 | 11 | 15 | 4 | 9 | 3 |
| Mean | −1.20 | −1.64 | −1.93 | −1.58 | −0.93 | −2.00 |
| (SD) | (0.994) | (0.912) | (1.476) | (0.788) | (0.997) | (1.000) |

Percent change from baseline to Day 14/EOT

| | | | | | | |
|---|---|---|---|---|---|---|
| n | 29 | 11 | 15 | 4 | 9 | 3 |
| Mean | −48.88 | −68.35 | −62.56 | −73.96 | −41.32 | −66.67 |
| (SD) | (37.271) | (38.399) | (42.230) | (32.342) | (43.189) | (33.333) |

ANMS GCSI-DD early satiety subscale score
Baseline

| | | | | | | |
|---|---|---|---|---|---|---|
| n | 30 | 11 | 15 | 4 | 9 | 3 |
| Mean | 2.67 | 2.70 | 2.64 | 2.50 | 2.63 | 3.22 |
| (SD) | (0.783) | (0.722) | (0.556) | (0.430) | (0.873) | (0.694) |

Day 14/EOT

| | | | | | | |
|---|---|---|---|---|---|---|
| n | 29 | 11 | 15 | 4 | 9 | 3 |
| Mean | 1.62 | 1.55 | 1.20 | 1.00 | 1.78 | 1.67 |
| (SD) | (1.015) | (0.934) | (0.941) | (0.816) | (1.093) | (0.577) |

Change from baseline to Day 14/EOT

| | | | | | | |
|---|---|---|---|---|---|---|
| n | 29 | 11 | 15 | 4 | 9 | 3 |
| Mean | −1.08 | −1.15 | −1.44 | −1.50 | −0.85 | −1.56 |
| (SD) | (0.834) | (0.911) | (0.948) | (1.139) | (0.852) | (0.509) |

Percent change from baseline to Day 14/EOT

| | | | | | | |
|---|---|---|---|---|---|---|
| n | 29 | 11 | 15 | 4 | 9 | 3 |
| Mean | −40.56 | −40.56 | −54.88 | −55.95 | −30.65 | −48.61 |
| (SD) | (31.248) | (41.704) | (35.651) | (41.582) | (35.876) | (14.633) |

ANMS GCSI-DD postprandial fullness subscale score
Baseline

| | | | | | | |
|---|---|---|---|---|---|---|
| n | 30 | 11 | 15 | 4 | 9 | 3 |
| Mean | 3.01 | 3.00 | 2.84 | 2.75 | 2.93 | 3.44 |
| (SD) | (0.634) | (0.471) | (0.795) | (0.167) | (0.760) | (0.509) |

Day 14/EOT

| | | | | | | |
|---|---|---|---|---|---|---|
| n | 29 | 11 | 15 | 4 | 9 | 3 |
| Mean | 1.79 | 1.73 | 1.53 | 1.25 | 2.22 | 2.00 |
| (SD) | (1.082) | (1.191) | (1.125) | (0.957) | (1.302) | (1.000) |

Change from baseline to Day 14/EOT

| | | | | | | |
|---|---|---|---|---|---|---|
| n | 29 | 11 | 15 | 4 | 9 | 3 |
| Mean | −1.24 | −1.27 | −1.31 | −1.50 | −0.70 | −1.44 |
| (SD) | (0.917) | (1.114) | (1.116) | (1.106) | (1.033) | (1.262) |

Percent change from baseline to Day 14/EOT

| | | | | | | |
|---|---|---|---|---|---|---|
| n | 29 | 11 | 15 | 4 | 9 | 3 |
| Mean | −41.52 | −42.90 | −45.50 | −53.13 | −26.11 | −40.00 |
| (SD) | (33.646) | (37.929) | (38.815) | (35.904) | (38.424) | (36.056) |

ANMS GCSI-DD upper abdominal pain subscale score
Baseline

| | | | | | | |
|---|---|---|---|---|---|---|
| n | 30 | 11 | 15 | 4 | 9 | 3 |
| Mean | 1.79 | 1.94 | 1.96 | 2.08 | 2.19 | 2.11 |
| (SD) | (1.160) | (0.841) | (0.872) | (0.833) | (1.042) | (1.171) |

Day 14/EOT

| | | | | | | |
|---|---|---|---|---|---|---|
| n | 29 | 11 | 15 | 4 | 9 | 3 |
| Mean | 0.97 | 0.73 | 0.87 | 1.00 | 1.33 | 1.33 |
| (SD) | (0.906) | (0.786) | (0.743) | (1.155) | (1.414) | (1.155) |

Change from baseline to Day 14/EOT

| | | | | | | |
|---|---|---|---|---|---|---|
| n | 29 | 11 | 15 | 4 | 9 | 3 |
| Mean | −0.80 | −1.21 | −1.09 | −1.08 | −0.85 | −0.78 |
| (SD) | (1.093) | (0.981) | (1.065) | (0.957) | (0.988) | (0.694) |

Percent change from baseline to Day 14/EOT

| | | | | | | |
|---|---|---|---|---|---|---|
| n | 26 | 11 | 15 | 4 | 8 | 3 |
| Mean | −50.43 | −59.74 | −41.03 | −58.33 | −43.15 | −46.67 |
| (SD) | (47.000) | (44.230) | (79.068) | (50.000) | (45.854) | (50.332) |

ANMS GCSI-DD vomiting subscale score
Baseline

| | | | | | | |
|---|---|---|---|---|---|---|
| n | 30 | 11 | 15 | 4 | 9 | 3 |
| Mean | 0.71 | 0.64 | 0.53 | 1.25 | 0.44 | 1.00 |
| (SD) | (1.020) | (1.206) | (0.764) | (0.918) | (0.577) | (1.000) |

Day 14/EOT

| | | | | | | |
|---|---|---|---|---|---|---|
| n | 29 | 11 | 15 | 4 | 9 | 3 |
| Mean | 0.14 | 0.36 | 0.07 | 0.00 | 0.33 | 0.00 |
| (SD) | (0.351) | (1.206) | (0.258) | (0.000) | (1.000) | (0.000) |

Change from baseline to Day 14/EOT

| | | | | | | |
|---|---|---|---|---|---|---|
| n | 29 | 11 | 15 | 4 | 9 | 3 |
| Mean | −0.59 | −0.27 | −0.47 | −1.25 | −0.11 | −1.00 |
| (SD) | (0.937) | (0.467) | (0.627) | (0.918) | (0.645) | (1.000) |

Percent change from baseline to Day 14/EOT

| | | | | | | |
|---|---|---|---|---|---|---|
| n | 16 | 5 | 8 | 4 | 5 | 2 |
| Mean | −71.73 | −80.00 | −94.64 | −100.00 | −64.00 | −100.00 |
| (SD) | (75.756) | (44.721) | (15.152) | (0.000) | (80.498) | (0.000) |

Baseline was defined as the mean score for the 3 days preceding randomization.
Day 14/EOT was defined as the last available score on or before the last date of treatment.
The ANMS GCSI-DD total score was the mean of the mean of the 5 subscale scores for severity of nausea, early satiety, postprandial fullness, upper abdominal pain, and vomiting.

### Patient Assessment of Gastrointestinal Disorders Symptom Severity Index

Table 12 presents the PAGI-SYM total and subscale scores and change and percent change from baseline to Day 14 for the Safety Population. For all deudomperidone treatment groups, PAGI-SYM total and subscale scores decreased from baseline at Day 14. Subjects receiving placebo also experienced decreases in PAGI-SYM total and subscale scores.

TABLE 12
Patient Assessment of Gastrointestinal Disorders Symptom Severity
Index Questionnaire Total and Subscale Scores - Safety Population

| | Cohort 1 | | Cohort 2 | | Cohort 3 | |
|---|---|---|---|---|---|---|
| | Deudomperi- | | Deudomperi- | | Deudomperi- | |
| Parameter | done | | done | | done | |
| Visit | 10 mg BID | Placebo | 20 mg QD | Placebo | 5 mg BID | Placebo |
| Statistic | (N = 30) | (N = 11) | (N = 15) | (N = 4) | (N = 9) | (N = 3) |

PAGI-SYM total score
Baseline

| | | | | | | |
|---|---|---|---|---|---|---|
| n | 29 | 11 | 15 | 4 | 9 | 3 |
| Mean (SD) | 2.94 | 3.25 | 2.72 | 3.50 | 3.37 | 2.86 |
| | (0.623) | (0.495) | (0.838) | (0.241) | (0.658) | (0.198) |

Day 14

| | | | | | | |
|---|---|---|---|---|---|---|
| n | 30 | 11 | 15 | 4 | 9 | 3 |
| Mean (SD) | 1.97 | 1.75 | 1.80 | 1.60 | 2.02 | 1.59 |
| | (0.855) | (0.949) | (1.251) | (1.329) | (1.300) | (0.638) |

Change from baseline to Day 14

| | | | | | | |
|---|---|---|---|---|---|---|
| n | 29 | 11 | 15 | 4 | 9 | 3 |
| Mean (SD) | −0.97 | −1.50 | −0.93 | −1.90 | −1.35 | −1.27 |
| | (0.990) | (1.312) | (0.806) | (1.286) | (1.179) | (0.660) |

Percent change from baseline to Day 14

| | | | | | | |
|---|---|---|---|---|---|---|
| n | 29 | 11 | 15 | 4 | 9 | 3 |
| Mean (SD) | −31.18 | −42.32 | −39.03 | −54.64 | −40.13 | −44.16 |
| | (33.026) | (35.140) | (33.180) | (37.449) | (32.850) | (21.761) |

PAGI-SYM heartburn/regurgitation subscale score
Baseline

| | | | | | | |
|---|---|---|---|---|---|---|
| n | 29 | 11 | 15 | 4 | 9 | 3 |
| Mean (SD) | 2.67 | 3.09 | 2.68 | 4.04 | 3.63 | 2.90 |
| | (1.126) | (0.867) | (1.179) | (0.410) | (0.783) | (1.110) |

Day 14

| | | | | | | |
|---|---|---|---|---|---|---|
| n | 30 | 11 | 15 | 4 | 9 | 3 |
| Mean (SD) | 1.62 | 1.70 | 1.62 | 1.79 | 1.84 | 1.10 |
| | (1.234) | (1.045) | (1.400) | (0.979) | (1.302) | (0.436) |

Change from baseline to Day 14

| | | | | | | |
|---|---|---|---|---|---|---|
| n | 29 | 11 | 15 | 4 | 9 | 3 |
| Mean (SD) | −1.07 | −1.39 | −1.06 | −2.25 | −1.79 | −1.81 |
| | (1.485) | (1.659) | (1.357) | (1.247) | (1.183) | (1.164) |

Percent change from baseline to Day 14

| | | | | | | |
|---|---|---|---|---|---|---|
| n | 29 | 11 | 15 | 4 | 9 | 3 |
| Mean (SD) | −25.45 | −16.23 | −34.56 | −54.50 | −50.23 | −59.68 |
| | (69.985) | (121.734) | (78.627) | (27.174) | (29.032) | (18.912) |

PAGI-SYM fullness/early satiety subscale score
Baseline

| | | | | | | |
|---|---|---|---|---|---|---|
| n | 29 | 11 | 15 | 4 | 9 | 3 |
| Mean (SD) | 3.60 | 3.70 | 3.42 | 3.63 | 3.89 | 3.50 |
| | (0.792) | (0.714) | (0.859) | (0.777) | (0.782) | (0.250) |

Day 14

| | | | | | | |
|---|---|---|---|---|---|---|
| n | 30 | 11 | 15 | 4 | 9 | 3 |
| Mean (SD) | 2.71 | 2.27 | 2.32 | 1.75 | 2.36 | 2.25 |
| | (1.109) | (1.222) | (1.321) | (1.173) | (1.426) | (1.090) |

Change from baseline to Day 14

| | | | | | | |
|---|---|---|---|---|---|---|
| n | 29 | 11 | 15 | 4 | 9 | 3 |
| Mean (SD) | −0.88 | −1.43 | −1.10 | −1.88 | −1.53 | −1.25 |
| | (0.915) | (1.347) | (1.224) | (1.665) | (1.422) | (1.090) |

Percent change from baseline to Day 14

| | | | | | | |
|---|---|---|---|---|---|---|
| n | 29 | 11 | 15 | 4 | 9 | 3 |
| Mean (SD) | −24.08 | −37.09 | −31.45 | −47.16 | −38.04 | −35.73 |
| | (27.288) | (34.721) | (36.563) | (45.857) | (37.136) | (30.941) |

PAGI-SYM nausea/vomiting subscale score
Baseline

| | | | | | | |
|---|---|---|---|---|---|---|
| n | 29 | 11 | 15 | 4 | 9 | 3 |
| Mean (SD) | 2.75 | 2.97 | 2.49 | 3.08 | 2.74 | 2.11 |
| | (1.022) | (0.640) | (0.765) | (0.957) | (0.997) | (1.171) |

Day 14

| | | | | | | |
|---|---|---|---|---|---|---|
| n | 30 | 11 | 15 | 4 | 9 | 3 |
| Mean (SD) | 1.78 | 1.09 | 1.29 | 1.08 | 1.74 | 0.89 |
| | (1.105) | (0.967) | (1.167) | (1.424) | (1.498) | (0.509) |

Change from baseline to Day 14

| | | | | | | |
|---|---|---|---|---|---|---|
| n | 29 | 11 | 15 | 4 | 9 | 3 |
| Mean (SD) | −0.93 | −1.88 | −1.20 | −2.00 | −1.00 | −1.22 |
| | (1.096) | (1.195) | (1.030) | (1.247) | (1.155) | (1.678) |

Percent change from baseline to Day 14

| | | | | | | |
|---|---|---|---|---|---|---|
| n | 29 | 11 | 15 | 4 | 9 | 3 |
| Mean (SD) | −30.39 | −60.70 | −50.18 | −69.55 | −39.58 | −35.56 |
| | (46.871) | (37.490) | (45.677) | (39.091) | (33.284) | (62.923) |

PAGI-SYM bloating subscale score
Baseline

| | | | | | | |
|---|---|---|---|---|---|---|
| n | 29 | 11 | 15 | 4 | 9 | 3 |
| Mean (SD) | 3.84 | 4.09 | 3.57 | 4.00 | 3.67 | 3.83 |
| | (1.001) | (0.491) | (1.387) | (0.000) | (0.612) | (0.289) |

Day 14

| | | | | | | |
|---|---|---|---|---|---|---|
| n | 30 | 11 | 15 | 4 | 9 | 3 |
| Mean (SD) | 2.65 | 2.45 | 2.47 | 2.25 | 2.67 | 2.67 |
| | (1.314) | (1.604) | (1.620) | (1.500) | (1.658) | (1.258) |

Change from baseline to Day 14

| | | | | | | |
|---|---|---|---|---|---|---|
| n | 29 | 11 | 15 | 4 | 9 | 3 |
| Mean (SD) | −1.17 | −1.64 | −1.10 | −1.75 | −1.00 | −1.17 |
| | (1.227) | (1.629) | (1.213) | (1.500) | (1.275) | (1.258) |

Percent change from baseline to Day 14

| | | | | | | |
|---|---|---|---|---|---|---|
| n | 29 | 11 | 14 | 4 | 9 | 3 |
| Mean (SD) | −26.78 | −39.77 | −36.38 | −43.75 | −30.16 | −30.36 |
| | (39.490) | (36.291) | (37.429) | (37.500) | (39.260) | (31.288) |

PAGI-SYM upper abdominal pain subscale score
Baseline

| | | | | | | |
|---|---|---|---|---|---|---|
| n | 29 | 11 | 15 | 4 | 9 | 3 |
| Mean (SD) | 2.74 | 3.00 | 2.27 | 3.38 | 3.17 | 2.67 |
| | (1.286) | (1.245) | (1.413) | (0.250) | (1.479) | (0.577) |

Day 14

| | | | | | | |
|---|---|---|---|---|---|---|
| n | 30 | 11 | 15 | 4 | 9 | 3 |
| Mean (SD) | 1.82 | 1.64 | 1.57 | 1.25 | 2.06 | 1.33 |
| | (1.323) | (1.120) | (1.450) | (1.500) | (1.530) | (1.155) |

Change from baseline to Day 14

| | | | | | | |
|---|---|---|---|---|---|---|
| n | 29 | 11 | 15 | 4 | 9 | 3 |
| Mean (SD) | −0.97 | −1.36 | −0.70 | −2.13 | −1.11 | −1.33 |
| | (1.395) | (1.614) | (0.978) | (1.377) | (1.635) | (0.577) |

Percent change from baseline to Day 14

| | | | | | | |
|---|---|---|---|---|---|---|
| n | 27 | 10 | 13 | 4 | 8 | 3 |
| Mean (SD) | −24.47 | −50.24 | −32.81 | −64.29 | −37.28 | −55.56 |
| | (80.505) | (34.116) | (41.891) | (42.857) | (43.374) | (38.490) |

PAGI-SYM lower abdominal pain subscale score
Baseline

| | | | | | | |
|---|---|---|---|---|---|---|
| n | 29 | 11 | 15 | 4 | 9 | 3 |
| Mean (SD) | 2.02 | 2.64 | 1.93 | 2.88 | 3.11 | 2.17 |
| | (1.306) | (1.164) | (1.624) | (0.250) | (1.537) | (1.258) |

Day 14

| | | | | | | |
|---|---|---|---|---|---|---|
| n | 30 | 11 | 15 | 4 | 9 | 3 |
| Mean (SD) | 1.25 | 1.36 | 1.53 | 1.50 | 1.44 | 1.33 |
| | (1.271) | (1.164) | (1.356) | (1.732) | (1.424) | (1.155) |

Change from baseline to Day 14

| | | | | | | |
|---|---|---|---|---|---|---|
| n | 29 | 11 | 15 | 4 | 9 | 3 |
| Mean (SD) | −0.79 | −1.27 | −0.40 | −1.38 | −1.67 | −0.83 |
| | (1.888) | (1.808) | (1.021) | (1.601) | (1.581) | (0.764) |

Percent change from baseline to Day 14

| | | | | | | |
|---|---|---|---|---|---|---|
| n | 24 | 11 | 11 | 4 | 8 | 3 |
| Mean (SD) | −38.38 | −25.49 | −10.91 | −50.00 | −52.08 | −47.62 |
| | (71.677) | (82.970) | (56.137) | (57.735) | (37.456) | (50.170) |

Baseline was defined as the last available assessment prior to the first dose of study drug.

### Clinical Grading Assessment Scale

Table 13 presents the responses for the Clinical Grading Assessment Scale for the ITT Population. Improvement was noted for the majority of subjects across all treatment groups.

TABLE 13
Clinical Grading Assessment Scale, Day 14 - Intent-to-Treat Population

| | Cohort 1 | | Cohort 2 | | Cohort 3 | |
|---|---|---|---|---|---|---|
| | Deudomperi- | | Deudomperi- | | Deudomperi- | |
| Respondent | done | | done | | done | |
| Parameter | 10 mg | | 20 mg | | 5 mg | |
| Category/ | BID | Placebo | QD | Placebo | BID | Placebo |
| Statistic | (N = 30) | (N = 11) | (N = 15) | (N = 4) | (N = 9) | (N = 3) |
| Subject | N′ = 23 | N′ = 9 | N′ = 14 | N′ = 4 | N′ = 9 | N′ = 3 |

Response category, n (%)

| | | | | | | |
|---|---|---|---|---|---|---|
| Improved | 17 (73.9) | 6 (66.7) | 9 (64.3) | 3 (75.0) | 5 (55.6) | 2 (66.7) |
| No change | 5 (21.7) | 2 (22.2) | 5 (35.7) | 1 (25.0) | 4 (44.4) | 1 (33.3) |
| Worsened | 1 (4.3) | 1 (11.1) | 0 (0.0) | 0 (0.0) | 0 (0.0) | 0 (0.0) |

CPGAS score

| | | | | | | |
|---|---|---|---|---|---|---|
| n | 23 | 9 | 14 | 4 | 9 | 3 |
| Mean (SD) | 3.0 (2.70) | 2.0 (3.43) | 2.9 (2.62) | 3.8 (2.63) | 2.1 (2.26) | 3.3 (3.06) |
| Investigator | N′ = 30 | N′ = 11 | N′ = 15 | N′ = 4 | N′ = 9 | N′ = 3 |

Response category, n (%)

| | | | | | | |
|---|---|---|---|---|---|---|
| Improved | 22 (73.3) | 8 (72.7) | 11 (73.3) | 3 (75.0) | 6 (66.7) | 2 (66.7) |
| No change | 8 (26.7) | 3 (27.3) | 4 (26.7) | 1 (25.0) | 3 (33.3) | 1 (33.3) |
| Worsened | 0 (0.0) | 0 (0.0) | 0 (0.0) | 0 (0.0) | 0 (0.0) | 0 (0.0) |

CPGAS score

| | | | | | | |
|---|---|---|---|---|---|---|
| n | 30 | 11 | 15 | 4 | 9 | 3 |
| Mean (SD) | 2.5 (1.98) | 2.6 (2.25) | 2.9 (2.26) | 3.3 (2.22) | 2.2 (2.05) | 3.3 (3.06) |

N′ = number of subjects with responses available at Day 14.
% = 100 × n/N′.
For assessment of global clinical response, the following question was utilized at Day 14: “In thinking about the last 2 weeks, how would you say your stomach/gastroparesis-related problems/symptoms have been compared to the period before you started treatment on this study?” Responses were the following: improved, no change, or worsened.
Subjects were asked to quantify their therapeutic response using the CPGAS.
Subjects picked a number over a range (+7 = completely better; 0 = no change; −7 = very much worse) that best answered the question.
The Investigator completed the same assessment based on their judgment of the subject's response to treatment.

### (ii) Pharmacodynamic Evaluation

Table 14 summarizes prolactin concentration and change from baseline by scheduled time point and treatment for the Safety Population. Prolactin concentration generally increased with dose and increased over the treatment period, confirming target engagement.

TABLE 14
Summary of Prolactin Concentration - Safety Population

| Parameter | Deudomperidone | | | |
|---|---|---|---|---|
| Visit | 10 mg BID | 20 mg QD | 5 mg BID | Pooled Placebo |
| Statistic | (N = 30) | (N = 15) | (N = 9) | (N = 18) |

Prolactin (mIU/L)
Baseline

| n | 30 | | 15 | | 9 | 18 | |
|---|---|---|---|---|---|---|---|
| Mean (SD) | 219.9 | (100.33) | 308.3 | (145.36) | 253.2 (123.43) | 244.7 | (91.91) |

Day 3

| n | 29 | | 15 | | 9 | 17 | |
|---|---|---|---|---|---|---|---|
| Mean (SD) | 570.2 | (461.62) | 830.5 | (432.63) | 396.7 (185.83) | 239.5 | (132.04) |

Change from baseline to Day 3

| n | 29 | | 15 | | 9 | 17 | |
|---|---|---|---|---|---|---|---|
| Mean (SD) | 351.5 | (427.84) | 522.1 | (380.00) | 143.4 (124.58) | −11.5 | (82.23) |

Prolactin (mIU/L)
Day 7

| n | 29 | | 15 | | 9 | 18 | |
|---|---|---|---|---|---|---|---|
| Mean (SD) | 640.0 | (548.03) | 1219.5 | (918.78) | 622.9 (317.61) | 244.6 | (103.57) |

Change from baseline to Day 7

| n | 29 | | 15 | | 9 | 18 | |
|---|---|---|---|---|---|---|---|
| Mean (SD) | 421.2 | (542.84) | 911.1 | (887.15) | 369.7 (263.19) | −0.2 | (60.33) |

Day 10

| n | 28 | | 15 | | 9 | 18 | |
|---|---|---|---|---|---|---|---|
| Mean (SD) | 734.9 | (603.97) | 1264.1 | (945.29) | 449.7 (147.64) | 270.2 | (126.10) |

Change from baseline to Day 10

| n | 28 | | 15 | | 9 | 18 | |
|---|---|---|---|---|---|---|---|
| Mean (SD) | 512.1 | (597.48) | 955.8 | (897.15) | 196.4 (99.96) | 25.5 | (103.20) |

Day 14

| n | 29 | | 15 | | 9 | 18 | |
|---|---|---|---|---|---|---|---|
| Mean (SD) | 774.2 | (547.70) | 1251.9 | (1155.35) | 578.0 (205.82) | 252.5 | (113.04) |

Change from baseline to Day 14

| n | 29 | | 15 | | 9 | 18 | |
|---|---|---|---|---|---|---|---|
| Mean (SD) | 555.5 | (545.40) | 943.6 | (1100.69) | 324.8 (187.12) | 7.8 | (65.82) |

Follow-up/early termination

| n | 2 | | — | — | — |
|---|---|---|---|---|---|
| Mean (SD) | 195.0 | (83.44) | — | — | — |

Change from baseline to follow-up/early termination

| n | 2 | | — | — | — |
|---|---|---|---|---|---|
| Mean (SD) | −32.5 | (47.38) | — | — | — |

Baseline was defined as the last measurement prior to the first dose of study drug.

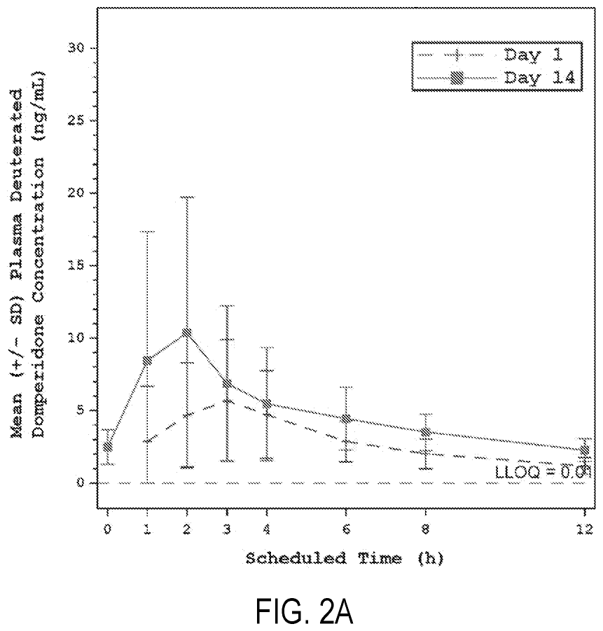

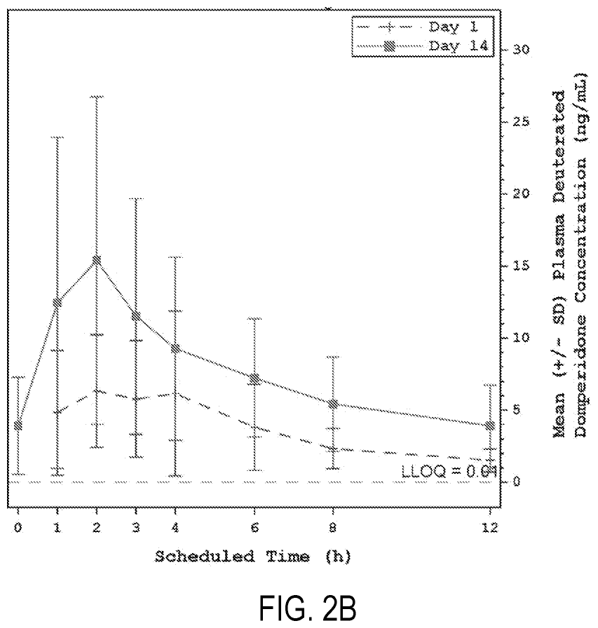

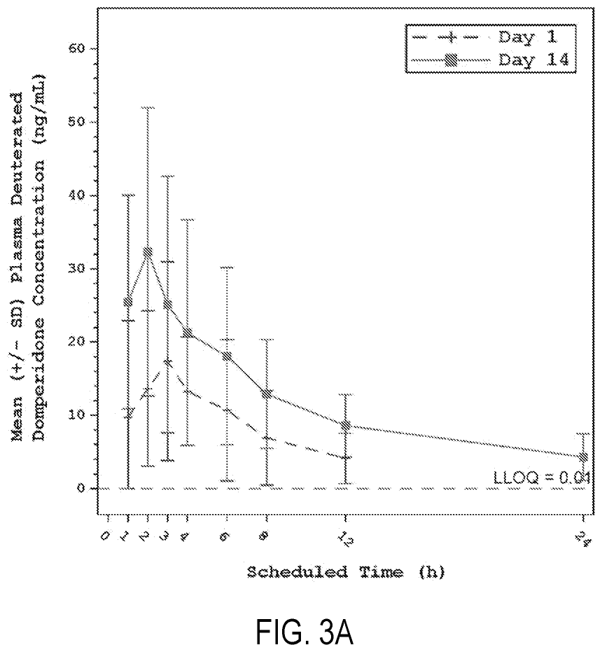

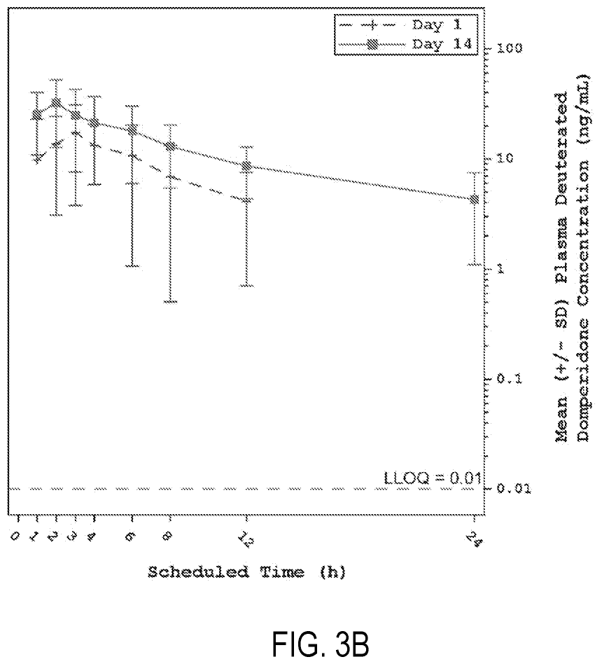

FIGS. 2A and 2B display the plot of mean (±SD) plasma deuterated domperidone concentrations versus time for the BID deudomperidone treatment groups over a single dosing interval following administration of deudomperidone on the morning of Day 1 and the morning of Day 14 on a linear scale for the P Population.

As expected, exposures to deuterated domperidone generally increased with increasing dose. Plasma concentrations of deuterated domperidone at or near peak levels were generally attained rapidly following administration of deudomperidone, with median Tₘₐₓ values ranging from 2.0 to 2.5 hours across dose groups. Plasma concentrations of deuterated domperidone generally declined from peak slowly. Table 15 summarizes the single dose (Day 1) and steady state (Day 14) plasma PR parameters of deuterated domperidone by treatment for the PR Evaluable Population.

TABLE 15
Summary of Single Dose (Day 1) and Steady State (Day 14) Pharmacokinetic Parameters
of Deuterated Domperidone - Pharmacokinetic Evaluable Population

| Day | | Deudomperidone | | |
|---|---|---|---|---|
| PK Parameter | Statistic | 10 mg BID | 20 mg QD | 5 mg BID |

Day 1

| Cₘₐₓ (ng/mL) | n | 23 | | 12 | | 6 | |
|---|---|---|---|---|---|---|---|
| | Mean (SD) | 10.066 | (5.6474) | 25.460 | (16.8212) | 6.730 | (4.4942) |
| | Geometric CV % | 60.3 | | 91.2 | | 99.3 | |
| Tₘₐₓ (h) | n | 23 | | 12 | | 6 | |
| | Median | 2.000 | (0.98, 6.00) | 2.500 | (1.00, 6.00) | 2.000 | (0.98, 4.05) |
| | (min, max) | | | | | | |
| AUC₀₋₁₂ | n | 16 | | 10 | | 3 | |
| (h*ng/mL) | Mean (SD) | 46.98 | (22.861) | 104.33 | (52.267) | 34.73 | (20.907) |
| | Geometric CV % | 52.2 | | 66.7 | | 62.7 | |
| AUC₀₋₂₄ | n | — | | 7 | | — | |
| (h*ng/mL) | Mean (SD) | — | | 148.39 | (46.885) | — | |
| | Geometric CV % | — | | 36.0 | | — | |
| | Day 14 | | | | | | |
| Cₘₐₓ (ng/mL) | n | 27 | | 15 | | 9 | |
| | Mean (SD) | 18.176 | (12.0178) | 36.407 | (18.9431) | 12.276 | (10.9355) |
| | Geometric CV % | 99.9 | | 57.3 | | 136.5 | |
| Tₘₐₓ (h) | n | 27 | | 15 | | 9 | |
| | Median | 2.000 | (0.97, 4.52) | 2.000 | (1.00, 6.00) | 2.000 | (1.00, 8.00) |
| | (min, max) | | | | | | |
| AUCₜₐᵤ | n | 21 | | 12 | | 7 | |
| (h*ng/mL) | Mean (SD) | 100.18 | (59.796) | 301.94 | (149.499) | 65.73 | (34.822) |
| | Geometric CV % | 60.0 | | 45.1 | | 66.8 | |
| AUC₀₋ₜ | n | 21 | | 12 | | 7 | |
| (h*ng/mL) | Mean (SD) | 180.63 | (134.906) | 378.55 | (179.858) | 113.24 | (49.668) |
| | Geometric CV % | 63.3 | | 45.4 | | 47.9 | |
| AUC_{0-∞} | n | 18 | | 11 | | 5 | |
| (h*ng/mL) | Mean (SD) | 179.31 | (95.721) | 430.68 | (202.717) | 118.71 | (47.618) |
| | Geometric CV % | 52.0 | | 46.3 | | 43.9 | |
| AUC_{% extrap} (%) | n | 18 | | 11 | | 5 | |
| | Mean (SD) | 9.31 | (4.521) | 8.48 | (4.413) | 11.72 | (4.472) |
| T½ (h) | n | 19 | | 11 | | 5 | |
| | Mean (SD) | 16.836 | (5.5237) | 15.855 | (3.2113) | 17.337 | (3.4695) |
| CL/F (L/h) | n | 18 | | 11 | | 5 | |
| | Mean (SD) | 69.640 | (30.1701) | 55.317 | (23.0233) | 48.394 | (20.4925) |
| Vz/F (L) | n | 18 | | 11 | | 5 | |
| | Mean (SD) | 1659.907 | (867.0031) | 1249.562 | (501.1720) | 1269.786 | (703.4723) |
| λz (1/h) | n | 19 | | 11 | | 5 | |
| | Mean (SD) | 0.0454 | (0.01460) | 0.0454 | (0.00941) | 0.0413 | (0.00851) |
| R_{Cmax} | n | 23 | | 12 | | 6 | |
| | Mean (SD) | 2.179 | (1.1530) | 2.620 | (2.5801) | 1.838 | (0.4992) |
| R_{AUC} | n | 16 | | 7 | | 3 | |
| | Mean (SD) | 2.430 | (1.1554) | 2.011 | (0.9469) | 1.653 | (0.1622) |

For sparse sampling profiles, only Cₘₐₓ, Tₘₐₓ, and R_{Cmax} were calculated. For profiles where no valid pre-dose sample was collected (missing or collected after dosing), only Cₘₐₓ, Tₘₐₓ, λ_{z}, T_{1/2}, and R_{Cmax} were calculated. Geometric CV % = 100 × (exp(SD²) − 1)^{0.5}, where SD is the standard deviation of the logarithm-transformed data. Tau = 12 hours for BID dosing and 24 hours for QD dosing.

At steady state, exposure (as assessed based on Cₘₐₓ and AUC) was approximately 2.0- to 2.5-fold higher than after both a single dose following BID dosing and a single dose following QD dosing. Variability at steady state was moderate to high, with geometric CV % ranging from approximately 43.9% to 136.5% for Cₘₐₓ and AUC (AUCₜₐᵤ, AUC₀₋ₜ, and AUC_{0-∞}) values. This may be, in part, due to the more truncated and/or variable sampling schedule in the current study.

Of note, there were 2 subjects who had plasma concentration values on Day 14 that were not consistent with what would be expected based on their assigned dosing regimen, suggesting potential lack of adherence. PK parameters are similar with and without these subjects, indicating that their presence in the dataset had no noteworthy impact on overall conclusions.

(iii) Efficacy, Pharmacodynamic, and Pharmacokinetic Conclusions

Gastric emptying time was reduced from baseline for subjects with idiopathic or diabetic gastroparesis treated with deudomperidone over a range of doses and for subjects treated with placebo.

Improvement in symptoms of gastroparesis was demonstrated for subjects receiving deudomperidone as well as placebo, as indicated by lower scores on the ANMS GCSI-DD (protocol-specified scoring method and manual scoring method), lower scores on the PAGI-SYM, and improvement noted for the majority of subjects on the Clinical Grading Assessment Scale.

Prolactin concentration generally increased with dose and increased over the treatment period, confirming target engagement.

The PK profile of deuterated domperidone following multiple doses of deudomperidone QD or BID to patients with gastroparesis is characterized by a relatively rapid Cₘₐₓ and long T %2. Steady-state accumulation ranged from approximately 2.0- to 2.5-fold higher than after a single dose following BID dosing and approximately 1.5- to 2.0-fold higher than after a single dose following QD dosing.

### H. Safety Results

(iii) Extent of Exposure

A total of 72 subjects were randomized to 1 of 3 cohorts. Fifty-four subjects were randomized to receive multiple doses of deudomperidone at 1 of the following dose levels: 10 mg BID, 20 mg QD, and 5 mg BID. The pooled placebo treatment group included 18 subjects. Overall, mean exposure to study drug was 14 days.

### (ii) Adverse Events

### Brief Summary of Adverse Events

Table 16 provides an overview of Aes by treatment at onset for the Safety Population.

There were no deaths, serious TEAEs, or discontinuations due to a TEAE. In total, 15 (20.8%) subjects experienced 23 TEAEs: 6 (20.1) subjects in the 10 mg BID deudomperidone treatment group (11 TEAEs reported), 6 (40.0%) subjects inthe 20 mg QD deudomperidone treatment group (8 TEAEs reported), 1 (11.1%) subject in the 5 mg BID deudomperidone treatment group (1 TEA reported), and 2 (11.1) subjects in the pooled placebo treatment group (3 TEAEs reported). The majority of TEAEs were mild in seventy.

Overall, 9 (12.5) subjects experienced a TEA that was considered related to study drug by the Investigator: 4 (13.3) subjects in the 10 mg BID deudomperidone treatment group, 4 (26.7) subjects inthe 20 mg QD deudomperidone treatment group, 1 (11.1) subject in the 5 mg BID deudomperidone treatment group, and 0 (0.0%) subjects in the pooled placebo treatment group.

TABLE 16
Overview of Adverse Events - Safety Population

| | Deudomperidone | | | | | | Pooled | | | |
|---|---|---|---|---|---|---|---|---|---|---|
| | 10 mg BID | | 20 mg QD | | 5 mg BID | | Placebo | | Total | |
| | (N = 30) | | (N = 15) | | (N = 9) | | (N = 18) | | (N = 72) | |
| Category | n (%) e | | n (%) e | | n (%) e | | n (%) e | | n (%) e | |
| Any Aes | 7 | (23.3) | 6 | (40.0) | 1 | (11.1) | 2 | (11.1) | 16 | (22.2) |
| Any TEAEs | 6 | (20.0) 11 | 6 | (40.0) 8 | 1 | (11.1) 1 | 2 | (11.1) 3 | 15 | (20.8) 23 |

Any TEAEs by maximum severity

| | | | | | | | | | | |
|---|---|---|---|---|---|---|---|---|---|---|
| Mild | 3 | (10.0) | 6 | (40.0) | 1 | (11.1) | 1 | (5.6) | 11 | (15.3) |
| Moderate | 2 | (6.7) | 0 | (0.0) | 0 | (0.0) | 1 | (5.6) | 3 | (4.2) |
| Severe | 1 | (3.3) | 0 | (0.0) | 0 | (0.0) | 0 | (0.0) | 1 | (1.4) |
| Any study drug-related | 4 | (13.3) | 4 | (26.7) | 1 | (11.1) | 0 | (0.0) | 9 | (12.5) |
| TEAEs | | | | | | | | | | |

Any study drug-related TEAEs by maximum severity

| | | | | | | | | | | |
|---|---|---|---|---|---|---|---|---|---|---|
| Mild | 4 | (13.3) | 4 | (26.7) | 1 | (11.1) | 0 | (0.0) | 9 | (12.5) |
| Moderate | 0 | (0.0) | 0 | (0.0) | 0 | (0.0) | 0 | (0.0) | 0 | (0.0) |
| Severe | 0 | (0.0) | 0 | (0.0) | 0 | (0.0) | 0 | (0.0) | 0 | (0.0) |
| Any serious TEAEs | 0 | (0.0) | 0 | (0.0) | 0 | (0.0) | 0 | (0.0) | 0 | (0.0) |
| Any study drug-related | 0 | (0.0) | 0 | (0.0) | 0 | (0.0) | 0 | (0.0) | 0 | (0.0) |
| serious TEAEs | | | | | | | | | | |
| Any TEAEs leading to | 0 | (0.0) | 0 | (0.0) | 0 | (0.0) | 0 | (0.0) | 0 | (0.0) |
| death | | | | | | | | | | |
| Any TEAEs leading to | 0 | (0.0) | 0 | (0.0) | 0 | (0.0) | 0 | (0.0) | 0 | (0.0) |
| discontinuation | | | | | | | | | | |
| Any study drug-related | 0 | (0.0) | 0 | (0.0) | 0 | (0.0) | 0 | (0.0) | 0 | (0.0) |

TEAEs leading to
discontinuation

% = 100 × n/N.
A TEAE was defined as an AE that started after the first dose of study drug.
Subjects reporting more than 1 AE were counted only once using the most severe incident.
Aes were coded using the Medical Dictionary for Regulatory Activities Version 22.0.

### Analysis of Adverse Events

Table 17 summarizes TEAEs by treatment at onset and by SOC and PT for the Safety Population. Overall, 15 (20.8%) subjects experienced a total of 23 TEAEs: 6 (20.0%) subjects who received 10 mg BID deudomperidone (11 TEAEs reported), 6 (40.0%) subjects who received 20 mg QD deudomperidone (8 TEAEs reported), and 1 (11.1%) subject who received 5 mg BID deudomperidone (1 TEAE reported). There were 2 (11.1%) subjects in the pooled placebo treatment group who experienced a total of 3 TEAEs.

The most common SOC of TEAEs was gastrointestinal disorders. In the deudomperidone treatment groups, 3 (10.0%) subjects who received 10 mg BID deudomperidone and 1 (6.7%) subject who received 20 mg QD deudomperidone experienced diarrhoea. The second most common SOCs of TEAEs were investigations and metabolism and nutrition disorders. In the deudomperidone treatment groups, 1 (3.3%) subject who received 10 mg BID deudomperidone and 2 (13.3%) subjects who received 20 mg QD deudomperidone experienced electrocardiogram QT prolonged, and 1 (3.3%) subject who received 10 mg BID deudomperidone and 1 (6.7%) subject who received 20 mg QD deudomperidone experienced hypoglycaemia. All other TEAEs in the deudomperidone treatment groups were experienced by 1 subject each: nausea, vomiting, blood creatine phosphokinase increased, decreased appetite, palpitations, and tinnitus (10 mg BID deudomperidone treatment group); constipation, dyspepsia, sinusitis, and headache (20 mg QD deudomperidone treatment group); and abdominal pain lower (5 mg BID deudomperidone treatment group).

In the pooled placebo treatment group, 1 (5.6%) subject each experienced nausea, arthralgia, and back pain.

TABLE 17
Summary of Treatment-Emergent Adverse Events by System
Organ Class and Preferred Term - Safety Population

| | Deudomperidone | | | | | | | | | |
|---|---|---|---|---|---|---|---|---|---|---|
| | 10 mg | | 20 mg | | 5 mg | | Pooled | | | |
| | BID | | QD | | BID | | Placebo | | Total | |
| System Organ Class | (N = 30) | | (N = 15) | | (N = 9) | | (N = 18) | | (N = 72) | |
| Preferred Term | n (%) e | | n (%) e | | n (%) e | | n (%) e | | n (%) e | |
| Subjects with any TEAEs | 6 | (20.0) 11 | 6 | (40.0) 8 | 1 | (11.1) 1 | 2 | (11.1) 3 | 15 | (20.8) 23 |
| Gastrointestinal disorders | 4 | (13.3) | 2 | (13.3) | 1 | (11.1) | 1 | (5.6) | 8 | (11.1) |
| Diarrhoea | 3 | (10.0) | 1 | (6.7) | 0 | (0.0) | 0 | (0.0) | 4 | (5.6) |
| Nausea | 1 | (3.3) | 0 | (0.0) | 0 | (0.0) | 1 | (5.6) | 2 | (2.8) |
| Abdominal pain lower | 0 | (0.0) | 0 | (0.0) | 1 | (11.1) | 0 | (0.0) | 1 | (1.4) |
| Constipation | 0 | (0.0) | 1 | (6.7) | 0 | (0.0) | 0 | (0.0) | 1 | (1.4) |
| Dyspepsia | 0 | (0.0) | 1 | (6.7) | 0 | (0.0) | 0 | (0.0) | 1 | (1.4) |
| Vomiting | 1 | (3.3) | 0 | (0.0) | 0 | (0.0) | 0 | (0.0) | 1 | (1.4) |
| Investigations | 2 | (6.7) | 2 | (13.3) | 0 | (0.0) | 0 | (0.0) | 4 | (5.6) |
| Electrocardiogram QT | 1 | (3.3) | 2 | (13.3) | 0 | (0.0) | 0 | (0.0) | 3 | (4.2) |
| prolonged | | | | | | | | | | |
| Blood creatine phosphokinase | 1 | (3.3) | 0 | (0.0) | 0 | (0.0) | 0 | (0.0) | 1 | (1.4) |
| increased | | | | | | | | | | |
| Metabolism and nutrition | 2 | (6.7) | 1 | (6.7) | 0 | (0.0) | 0 | (0.0) | 3 | (4.2) |
| disorders | | | | | | | | | | |
| Hypoglycaemia | 1 | (3.3) | 1 | (6.7) | 0 | (0.0) | 0 | (0.0) | 2 | (2.8) |
| Decreased appetite | 1 | (3.3) | 0 | (0.0) | 0 | (0.0) | 0 | (0.0) | 1 | (1.4) |
| Musculoskeletal and | 0 | (0.0) | 0 | (0.0) | 0 | (0.0) | 2 | (11.1) | 2 | (2.8) |
| connective tissue disorders | | | | | | | | | | |
| Arthralgia | 0 | (0.0) | 0 | (0.0) | 0 | (0.0) | 1 | (5.6) | 1 | (1.4) |
| Back pain | 0 | (0.0) | 0 | (0.0) | 0 | (0.0) | 1 | (5.6) | 1 | (1.4) |
| Cardiac disorders | 1 | (3.3) | 0 | (0.0) | 0 | (0.0) | 0 | (0.0) | 1 | (1.4) |
| Palpitations | 1 | (3.3) | 0 | (0.0) | 0 | (0.0) | 0 | (0.0) | 1 | (1.4) |
| Ear and labyrinth disorders | 1 | (3.3) | 0 | (0.0) | 0 | (0.0) | 0 | (0.0) | 1 | (1.4) |
| Tinnitus | 1 | (3.3) | 0 | (0.0) | 0 | (0.0) | 0 | (0.0) | 1 | (1.4) |
| Infections and infestations | 0 | (0.0) | 1 | (6.7) | 0 | (0.0) | 0 | (0.0) | 1 | (1.4) |
| Sinusitis | 0 | (0.0) | 1 | (6.7) | 0 | (0.0) | 0 | (0.0) | 1 | (1.4) |
| Nervous system disorders | 0 | (0.0) | 1 | (6.7) | 0 | (0.0) | 0 | (0.0) | 1 | (1.4) |
| Headache | 0 | (0.0) | 1 | (6.7) | 0 | (0.0) | 0 | (0.0) | 1 | (1.4) |

% = 100 × n/N.
A TEAE was defined as an AE that started after the first dose of study drug.
Subjects reporting more than 1 AE for a given MedDRA preferred term were counted only once for that term.
Subjects reporting more than 1 type of event within an SOC were counted only once for that SOC.
Aes were coded using the MedDRA Version 22.0.

### Drug-Related Adverse Events

Table 18 summarizes study drug-related TEAEs by treatment at onset and by SOC and PT for the Safety Population. Overall, 9 (12.5%) subjects experienced a total of 11 study drug-related TEAEs: 4 (13.3%) subjects who received 10 mg BID deudomperidone (6 TEAEs reported), 4 (26.7) subjects who received 20 mg QD deudomperidone (4 TEAEs reported), and 1 (11.1%) subject who received 5 mg BID deudomperidone (1 TEAE reported). No subjects in the pooled placebo treatment group experienced a study drug-related TEAE.

The most common SOC of study drug-related TEAEs was gastrointestinal disorders. In the deudomperidone treatment groups, 2 (6.70) subjects who received 10 mg BID deudomperidone and 1 (6.70) subject who received 20 mg QD deudomperidone experienced diarrhoea. The second most common SOC of study drug-related TEAEs was investigations. In the deudomperidone treatment groups, 2 (13.30) subjects who received 20 mg QD deudomperidone experienced electrocardiogram Q prolonged. All other study drug-related TEAEs in the deudomperidone treatment groups were experienced by 1 subject each: nausea, blood creatine phosphokinase increased, palpitations, and tinnitus (10 mg BID deudomperidone treatment group); headache (20 mg QD deudomperidone treatment group); and abdominal pain lower (5 mg BID deudomperidone treatment group).

TABLE 18
Summary of Study Drug-Related Treatment-Emergent Adverse Events
by System Organ Class and Preferred Term - Safety Population

| | Deudomperidone | | | | | | Pooled | | | |
|---|---|---|---|---|---|---|---|---|---|---|
| | 10 mg BID | | 20 mg QD | | 5 mg BID | | Placebo | | Total | |
| System Organ Class | (N = 30) | | (N = 15) | | (N = 9) | | (N = 18) | | (N = 72) | |
| Preferred Term | n (%) e | | n (%) e | | n (%) e | | n (%) e | | n (%) e | |
| Subjects with any | 4 | (13.3) 6 | 4 | (26.7) 4 | 1 | (11.1) 1 | 0 | (0.0) 0 | 9 | (12.5) 11 |
| study drug-related | | | | | | | | | | |
| TEAE | | | | | | | | | | |
| Gastrointestinal disorders | 3 | (10.0) | 1 | (6.7) | 1 | (11.1) | 0 | (0.0) | 5 | (6.9) |
| Diarrhoea | 2 | (6.7) | 1 | (6.7) | 0 | (0.0) | 0 | (0.0) | 3 | (4.2) |
| Abdominal pain lower | 0 | (0.0) | 0 | (0.0) | 1 | (11.1) | 0 | (0.0) | 1 | (1.4) |
| Nausea | 1 | (3.3) | 0 | (0.0) | 0 | (0.0) | 0 | (0.0) | 1 | (1.4) |
| Investigations | 1 | (3.3) | 2 | (13.3) | 0 | (0.0) | 0 | (0.0) | 3 | (4.2) |
| Electrocardiogram | 0 | (0.0) | 2 | (13.3) | 0 | (0.0) | 0 | (0.0) | 2 | (2.8) |
| QT prolonged | | | | | | | | | | |
| Blood creatine | 1 | (3.3) | 0 | (0.0) | 0 | (0.0) | 0 | (0.0) | 1 | (1.4) |
| phosphokinase increased | | | | | | | | | | |
| Cardiac disorders | 1 | (3.3) | 0 | (0.0) | 0 | (0.0) | 0 | (0.0) | 1 | (1.4) |
| Palpitations | 1 | (3.3) | 0 | (0.0) | 0 | (0.0) | 0 | (0.0) | 1 | (1.4) |
| Ear and labyrinth disorders | 1 | (3.3) | 0 | (0.0) | 0 | (0.0) | 0 | (0.0) | 1 | (1.4) |
| Tinnitus | 1 | (3.3) | 0 | (0.0) | 0 | (0.0) | 0 | (0.0) | 1 | (1.4) |
| Nervous system disorders | 0 | (0.0) | 1 | (6.7) | 0 | (0.0) | 0 | (0.0) | 1 | (1.4) |
| Headache | 0 | (0.0) | 1 | (6.7) | 0 | (0.0) | 0 | (0.0) | 1 | (1.4) |

% = 100 × n/N.
A TEAE was defined as an AE that started after the first dose of study drug.
Subjects reporting more than 1 AE for a given MedDRA preferred term were counted only once for that term.
Subjects reporting more than 1 type of event within an SOC were counted only once for that SOC.
Aes were coded using the MedDRA Version 22.0.

### Adverse Events by Severity

The majority of TEAEs were mild in severity. In total, 11 (15.3%) subjects reported a mild TEAE, 3 (4.2%) subjects reported a moderate TEAE, and 1 (1.4%) subject reported a severe TEAE. All study drug-related TEAEs were mild in severity.

There were no deaths, SAEs, or other significant Aes during the study.

(iii) Clinical Laboratory Evaluation

### Evaluation of Laboratory Parameters

Laboratory values over time: There were no meaningful changes from baseline in laboratory parameters. Prolactin concentration generally increased with dose and increased over the treatment period, confirming target engagement.

Individual subject changes: There were no clinically meaningful shifts in any laboratory parameters from baseline. There were no clinically meaningful shifts in prolactin concentration from baseline.

Individual clinically significant abnormalities: There were no laboratory abnormalities that were considered an SAE or resulted in discontinuation from the study or study drug.

### (iv) Vital Signs, Physical Examination Findings, and Other Observations Related to Safety

There were no clinically meaningful shifts in vital signs from baseline. There were no clinically meaningful shifts in physical examination results.

There were no clinically meaningful prolongations of the QTcF interval; however, some subjects met the protocol-specified alert criteria for QTcF. Two (13.3%) subjects in the 20 mg QD deudomperidone treatment group experienced a ≥6% change from baseline in QTcF on both Day 3 and Day 7. On Day 14 at 4 hours post-dose, 1 (3.6%) subject in the 10 mg BID deudomperidone treatment group, 3 (20.0%) subjects in the 20 mg QD deudomperidone treatment group, and 1 (6.3%) subject in the pooled placebo treatment group experienced a ≥6% change from baseline in QTcF. On Day 14 at 6 hours post-dose, 1 (4.3%) subject in the 10 mg BID deudomperidone treatment group, 2 (16.7%) subjects in the 20 mg QD deudomperidone treatment group, and 1 (7.1%) subject in the pooled placebo treatment group experienced a ≥6% change from baseline in QTcF. One (6.7%) female subject in the 20 mg QD deudomperidone treatment group experienced a QTcF ≤470 msec. Two subjects were assessed as having study drug-related TEAEs of electrocardiogram QT prolonged.

### Safety Conclusions

No subjects died during the study, experienced a serious TEAE, or discontinued from the study due to a TEAE. Overall, 15 (20.8%) subjects experienced a TEAE, and 9 (12.5%) subjects overall experienced a TEAE that was considered related to study drug by the Investigator; all of these subjects received deudomperidone. All study drug-related TEAEs were mild in severity. The most common SOC of study drug-related TEAEs was gastrointestinal disorders.

There were no clinically meaningful changes from baseline in laboratory parameters during the study, and there were no clinically meaningful changes in vital signs or physical examination results during the study. There were no clinically meaningful prolongations of the QTcF interval; however, 2 subjects met the protocol-specified alert criteria for QTcF. For 1 subject, the alert of ≥6% QTcF increase from baseline was at 4 and 6 hours post-dose on Day 14; notably, the QTcF never increased above 426 msec. For the second subject, the alert of ≥6% QTcF increase from baseline was on Day 7, with a value of 474 msec (baseline QTcF average was 430 msec); this subject continued on study drug and the QTcF remained at or below baseline for the remainder of the study.

### I. Discussion and Overall Conclusions

A total of 72 subjects were randomized to 1 of 3 cohorts: 54 subjects were randomized to receive multiple doses of deudomperidone at either 10 mg BID, 20 mg QD, or 5 mg BID, and the pooled placebo treatment group included 18 subjects. Overall, mean exposure to study drug was 14 days. For all treatment groups, the majority of subjects (51 [70.8%] subjects) had diabetic gastroparesis etiology, with similar numbers of diabetic subjects with and without neuropathy except for Cohort 2, where the large majority of subjects had neuropathy. No observed differences in demographic and baseline characteristics are considered likely to have influenced the data interpretation and conclusions. There were no issues with study drug administration, and there were no differences in mean percent adherence based on drug accountability between treatment groups.

Improvement in symptoms of gastroparesis was demonstrated for subjects receiving deudomperidone as well as placebo, as indicated by lower scores on the ANMS GCSI-DD (protocol-specified scoring method and manual scoring method), lower scores on the PAGI-SYM, and improvement noted for the majority of subjects on the Clinical Grading Assessment Scale. Separate meta-analyses have demonstrated positive associations between faster gastric emptying and improved upper gastrointestinal symptoms.

However, exposure to deuterated domperidone (as assessed based on Cₘₐₓ and AUC) in patients with gastroparesis is approximately 2-fold higher compared to that expected based on PK data in healthy subjects for a given dose level of deudomperidone, despite similar accumulation in both populations. Specifically, data in healthy subjects would indicate that the 10 mg deudomperidone BID dosing regimen would be expected to produce a mean Cₘₐₓ of approximately 7 to 11 ng/mL; however, Cₘₐₓ in patients with gastroparesis following administration of 10 mg deudomperidone BID is approximately 18 ng/mL. Similarly, an approximately 2-fold higher AUC was observed in patients with gastroparesis compared to what would be expected in healthy subjects. PK differences are most pronounced in patients with more severe gastroparesis.

Prolactin concentration generally increased with dose and increased over the treatment period, confirming target engagement.

Characterization of the safety of deudomperidone after multiple doses was also a study objective. Multiple doses of deudomperidone were generally well tolerated by the subjects with idiopathic or diabetic gastroparesis participating in this study. No subjects died during the study, experienced a serious TEAE, or discontinued from the study due to a TEAE. In total, 15 (20.8%) subjects experienced 23 TEAEs: 6 (20.0%) subjects in the 10 mg BID deudomperidone treatment group (11 TEAEs reported), 6 (40.0%) subjects in the 20 mg QD deudomperidone treatment group (8 TEAEs reported), (11.1%) subject in the 5 mg BID deudomperidone treatment group (1 TEAE reported), and (11.1%) subjects in the pooled placebo treatment group (3 TEAEs reported). The majority of TEAEs were mild in severity. Overall, 9 (12.5%) subjects experienced a TEAE that was considered related to study drug by the Investigator: 4 (13.3%) subjects in the 10 mg BID deudomperidone treatment group, 4 (26.7%) subjects in the 20 mg QD deudomperidone treatment group, 1 (11.1%) subject in the 5 mg BID deudomperidone treatment group, and 0 (0.0%) subjects in the pooled placebo treatment group. In the deudomperidone treatment groups, 2 (6.7%) subjects who received 10 mg BID deudomperidone and 1 (6.7%) subject who received 20 mg QD deudomperidone experienced diarrhoea, and 2 (13.3%) subjects who received 20 mg QD deudomperidone experienced electrocardiogram QT prolonged. All other study drug-related TEAEs in the deudomperidone treatment groups were experienced by 1 subject each: nausea, blood creatine phosphokinase increased, palpitations, and tinnitus (10 mg BID deudomperidone treatment group); headache (20 mg QD deudomperidone treatment group); and abdominal pain lower (5 mg BID deudomperidone treatment group). All study drug-related TEAEs were mild in severity. No subjects in the pooled placebo treatment group experienced a study drug-related TEAE.

There were no clinically meaningful changes from baseline in laboratory parameters during the study, and there were no clinically meaningful changes in vital signs or physical examination results during the study. There were no clinically meaningful prolongations of the QTcF interval; however, 2 subjects met the protocol-specified alert criteria for QTcF. These subjects experienced a TEAE of electrocardiogram QT prolonged that was considered related to study drug and that occurred well after Tₘₐₓ. For 1 subject, the alert of 6% QTcF increase from baseline was at 4 and 6 hours post-dose on Day 14; notably, the QTcF never increased above 426 msec. For the second subject, the alert of ≥6% QTcF increase from baseline was on Day 7, with a value of 474 msec (baseline QTcF average was 430 msec); this subject continued on study drug and the QTcF remained at or below baseline for the remainder of the study.

### Overall Conclusions

In this study, multiple, oral doses of deudomperidone of 10 mg BID, 20 mg BID, and 5 mg BID were generally well tolerated by adults with idiopathic or diabetic gastroparesis. There were no deaths, serious TEAEs, or discontinuations due to a TEAE. The majority of TEAEs were mild in severity.

Reduced gastric emptying time and improvement in symptoms of gastroparesis from baseline were observed for subjects with idiopathic or diabetic gastroparesis treated with deudomperidone over a range of doses and for subjects treated with placebo.

The PK profile of deuterated domperidone following multiple doses of deudomperidone QD or BID to patients with gastroparesis is characterized by a relatively rapid Cₘₐₓ and long T½. Steady-state accumulation ranged from approximately 2.0- to 2.5-fold higher than after a single dose following BID dosing and approximately 1.5- to 2.0-fold higher than after a single dose following QD dosing. Following administration of a given dose level of deudomperidone, exposure (as assessed based on Cₘₐₓ and AUC) in patients with gastroparesis is approximately 2-fold higher compared to that expected based on PK data in healthy subjects.

### Example 3: Components and Composition

Table 19 displays the composition of CIN-102 5 mg, 10 mg, and 20 mg soft-gel capsules. CIN-102 has been formulated with 5 mg, 10 mg, or 20 mg of CIN-102 drug substance (as a free base) and excipients intended to produce a lower Cₘₐₓ. Based on the lipophilic nature of the fill formulation, a 2A gelatin shell formula was used for encapsulation.

TABLE 19
Components of CIN-102 5 mg, 10 mg, and 20 mg Soft-Gel Capsules
Component

CIN-102 (5 mg, 10 mg, or 20 mg)
Medium chain triglycerides
Glyceryl distearate
Butylated hydroxyanisole
Butylated hydroxytoluene
Soft-gel capsule ¹
¹ The soft-gel capsule contains the following ingredients: gelatin 150 LB, NF; glycerin, USP; gelatin hydrolysate, NF; purified water, USP; titanium dioxide, USP; and FD&C blue #1.

Placebo soft-gel capsules contain all components listed in Table 19, with the exception of CIN-102.

### Example 4: Thorough QT Study

This example details the results of the cardiodynamic evaluation of CIN-102, in which a single dose of CIN-102 (producing therapeutic and supratherapeutic exposures), was administered to healthy adult subjects. The primary objective of this study was to assess the effect of CIN-102 on cardiac repolarization based on baseline and placebo-corrected change in corrected QT interval using Fridericia's formula (QTcF) in healthy subjects using a concentration-QTc analysis (primary analysis). Serial ECGs were extracted from continuous ECG recordings at prespecified time points starting at least one hour prior to dosing and ending approximately 48 hours post-dose in each period.

A 10 mg dose of deudomperidone drug product produces a Cₘₐₓ which is approximately 50% lower than the same oral dose domperidone in healthy subjects, Mean half-life values following a single dose of deudomperidone drug product range from approximately 18 to 29 hours across the dose range studied. In order to assess the effects of the deudomperidone drug product on QT interval a definitive TQT study was performed. Primary analysis was based on concentration-QTc modeling of the relationship between plasma drug concentrations and change-from-baseline QTcF (ΔQTcF) with the intent to exclude an effect of placebo-corrected ΔQTcF (ΔΔQTcF) >10 milliseconds (ms) at clinically relevant plasma levels.

The study was a randomized, partially double-blind, active- and placebo-controlled, single dose, four-period, crossover study in healthy subjects 18-55 years of age. During the study, each subject received a single dose of each of the following in a randomized fashion (1 treatment per study period) in separate periods (≥10 days between doses) as shown in Table 20:
30 mg deudomperidone formulation (representing approximate steady-state therapeutic exposures in patients):
100 mg deudomperidone formulation (representing potential steady-state supratherapeutic exposures in patients);
400 mg moxifloxacin; and
placebo.

TABLE 20
Treatment sequences to which subjects could be randomized

| Sequence | Period 1 | Period 2 | Period 3 | Period 4 |
|---|---|---|---|---|
| 1 | A | B | D | C |
| 2 | B | C | A | D |
| 3 | D | A | C | B |
| 4 | C | D | B | A |
| 5 | B | D | A | C |
| 6 | D | C | B | A |
| 7 | A | B | C | D |
| 8 | C | A | D | B |
| 9 | D | A | B | C |
| 10 | A | C | D | B |
| 11 | B | D | C | A |
| 12 | C | B | A | D |

For treatments A. B, and D, subjects ingested a total of 10 capsules of CIN-102 and/or matching placebo. For treatment C, subjects ingested a single 400 mg tablet of moxifloxacin.

Subjects fasted for a minimum of 8 hours before and 4 hours after administration of each treatment. There was a minimum 10-day washout between treatments.

### Study Objectives

The primary objective of this study was to assess the effect of CIN-102 on cardiac repolarization based on baseline and placebo-corrected change in corrected QT interval using Fridericia's formula (QTcF).

The secondary objectives of this study were:
To assess the effect of CIN-102 on other ECG parameters (heart rate [HR], PR and QRS intervals, and treatment-emergent changes of T-wave morphology)
To characterize the safety of single doses of CIN-102 when administered to healthy adult subjects
To characterize the pharmacokinetics (PK) of single doses of CIN-102 when administered to healthy adult subjects

### Primary Endpoint

The primary endpoint was placebo-corrected change-from-baseline QTcF (ΔΔQTcF).

The secondary endpoints were:
Change-from-baseline QTcF, HR, PR, QRS intervals (ΔQTcF, ΔHR, ΔPR, and ΔQRS)
Placebo-corrected change-from-baseline HR, PR, and QRS (ΔΔHR, ΔΔPR, ΔΔQRS)
Categorical outliers for QTcF, HR, PR, and QRS
Frequency of treatment-emergent changes of T-wave morphology and U-waves presence

### ECG and Pharmacokinetic Sample Collection

In each treatment period, continuous 12-lead ECGs were recorded starting at least one hour prior to dosing and ending approximately 48 hours post-dose. Twelve-lead ECGs were extracted in up to 10 replicates at the central ECG laboratory (ERT, Philadelphia, PA) at the following time points in each period: at 3 time points within one hour prior to dosing (−45, −30 and −15 minutes) and at 0.5, 1, 1.5, 2, 2.5, 3, 4, 5, 6, 8, 12, 24, 36, and 48 hours after each dose. Subjects were supinely resting for at least 10 minutes before and 5 minutes after each time point. When ECG extractions coincided with safety, ECGs, vital signs assessment, and blood draws, procedures were carried out in said order. No PK or other laboratory draws were performed and the subject was not approached until at least 5 minutes had elapsed after the 5-minute window for 12-lead ECG extraction for QT analysis.

Blood samples for PK determination were drawn at the same time points immediately after the ECG extractions.

ECG intervals were measured by the core laboratory in a blinded manner using the Early Precision QT technique (EPQT). The ECG database was locked before any statistical analysis was undertaken.

### TQT Plus ECG Extraction Technique

During protocol-specified ECG extraction windows, up to 10 non-overlapping 14-second digital 12-lead ECG tracings were extracted from continuous recordings. The TQT Plus method enabled the extraction of a high-quality data set by identifying periods of recordings with the lowest available HR variability and noise.

The ECGs were extracted according to the following principles:
The actual times of dosing, extraction windows, and PK sampling were communicated to the central ECG laboratory by the study center personnel.
The TQT Plus process identified periods of stable HR on the continuous 12-lead ECG tracing within the 5-minute extraction window. Stability was defined as variation in the HR and other ECG parameters from beat-to-beat lower than a predefined threshold. If the TQT Plus method resulted in a low number of consecutive, readable cardiac cycles in the 5-minute window, the time point was fully reviewed manually.
Replicate, non-overlapping ECGs were extracted in close succession within each extraction window.

### Early Precision QT Analysis

At each nominal time point specified in the protocol, up to 10 ECG replicates were extracted with TQT Plus methods. All readable cardiac cycles from these ECG replicates were assessed for multiple quality metrics, including beat stability, HR changes, noise, and other parameters, and were categorized into high and low confidence rank. All low confidence beats were fully reviewed and adjudicated manually by ECG technicians using pass-fail criteria. The beats found acceptable by manual review were included in the analysis.

The primary analysis lead was Lead II. If Lead II was not analyzable, then the primary lead of analysis was changed to V5 for the subject's entire data set. If Lead V5 was not analyzable, then Lead V2 was used for the subject's entire data set. If both alternate leads could not be used, the ERT cardiologist had the option to deem the data non-analyzable or could decide to use the most appropriate lead for the subject's entire data set. The lead used for analysis was listed in the data listings transferred from ERT.

Categorical T-wave morphology analysis and measurement of PR and QRS intervals were fully performed manually using a semi-automated process in 3 of the 10 ECG replicates at each time point. Final quality control and diagnostic interpretations were performed by the study cardiologist. When the results for each time point were compiled in the final raw data set, the average QTcF from the 3 manually reviewed ECGs was compared to the average QTcF from the TQT Plus ECG replicates (up to 10) for quality control purposes.

A total of 62 subjects were enrolled in the TQT study. At baseline, data were available from all 55 subjects in the placebo treatment period and then from 52 subjects in the deudomperidone formulation (30 mg) and supratherapeutic (100 mg) treatment periods and from 51 subjects in the moxifloxacin treatment period. The mean (SD) age of subjects was 38.5 (9.20) years, and the mean (SD) BMI of the subjects was 26.3 (2.48) kg/m². Sixty-one percent of the subjects were male and 39% were female. Baseline ECG parameters were within expectations for a healthy subject population.
(i) PK data from the study demonstrated that the highest mean plasma concentrations of deuterated domperidone were observed at 1.5 and 2 hours post-dose for the 30 and 100 mg treatment groups, respectively.

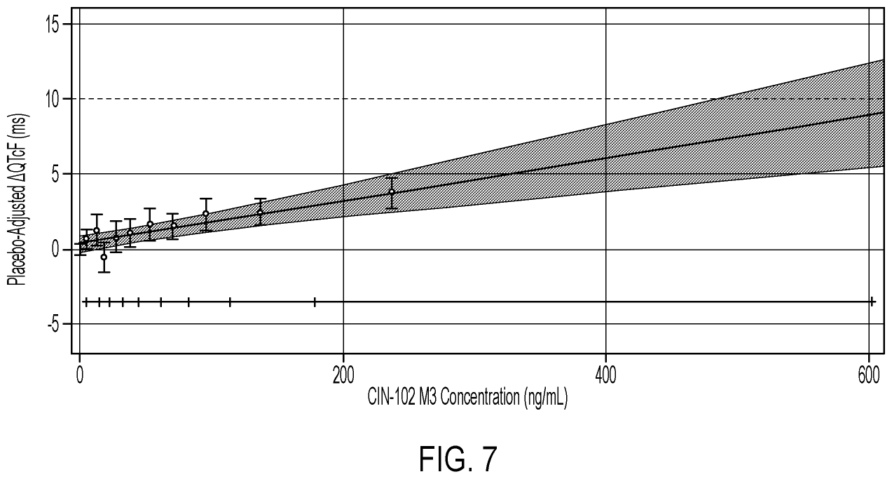

The highest mean plasma concentrations of M3 (the primary metabolite) were each observed at 2 hours post-dose for the 30 and 100 mg treatment groups.
LS mean placebo-corrected change from baseline in QTcF (ΔΔQTcF) within the first 4 hours on the deudomperidone formulation ranged from −0.3 to 1.8 ms at 0.5 and 2 hour(s) post-dose, respectively, in the 30 mg treatment group and from −0.1 to 3.7 ms at 0.5 and 3 hour(s) post-dose, respectively, in the 100 mg treatment group. See, FIG. 7. LS mean and 90% CI based on a linear mixed-effects model for active and placebo treatment groups:

$$Δ Q T c F = Time + Treatment + Time \times Treatment + Baseline Q T c F + Period + Sequence .$$

An unstructured covariance structure was used to specify the repeated measures at postdose time points for subject within treatment period. The model also included a subject-specific random effect.

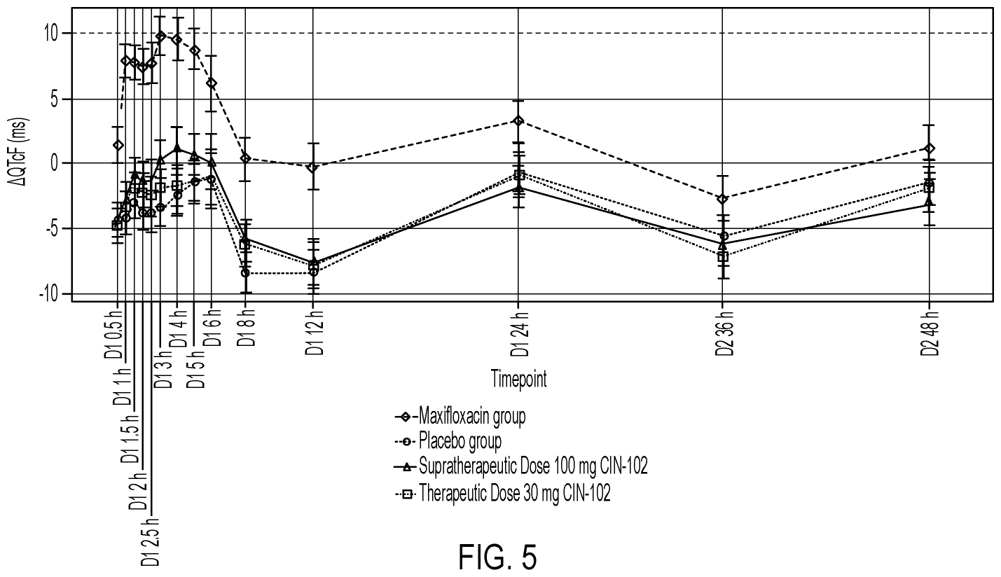

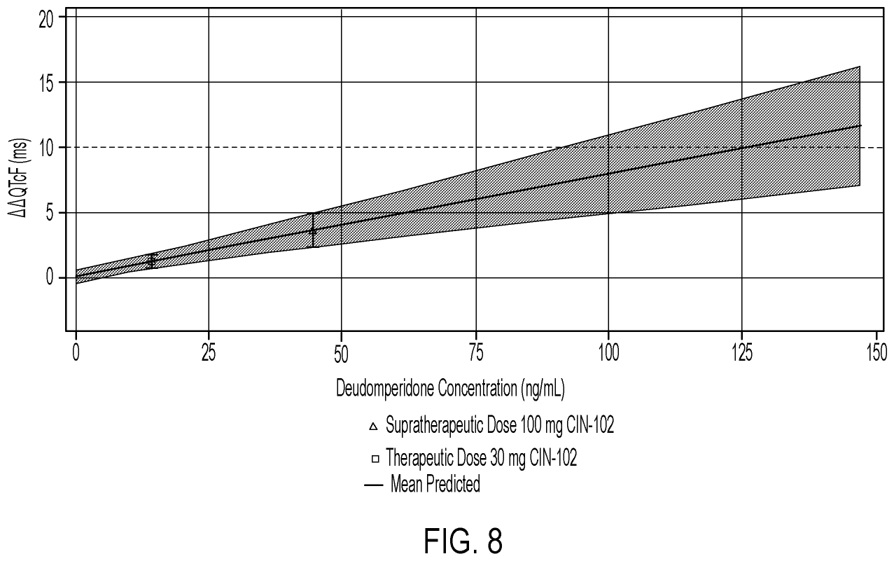

In FIG. 5, the upper bound of the 90% CI of ΔΔQTcF was <10 ms for both deudomperidone formulation doses at all time points, demonstrating that an effect on QTcF exceeding 10 ms could be excluded on both deudomperidone formulation doses. Moxifloxacin caused clear QTc prolongation.
(ii) Analyses were also undertaken to understand the relationship between plasma concentrations of deudomperidone and ΔΔQTcF, independent of time. Based on this concentration-QTc analysis, an effect on ΔΔQTcF exceeding 10 ms can be excluded up to deudomperidone plasma concentrations of ˜92 ng/mL which is approximately 5 times the therapeutic steady state concentration (FIG. 8). The effect on ΔΔQTcF can be predicted to 1.10 ms (90% CI: 0.58 to 1.61) and 3.50 ms (90% CI: 2.18 to 4.82) for the 30 and 100 mg treatment groups, respectively.

In FIG. 8, the solid black line with gray shaded area denotes the model-predicted mean (90% CI) ΔΔQTcF, which was calculated from the equation ΔΔQTcF=−0.02844 (ms)+0.7918 (ms per ng/mL)×deuterated domperidone concentration (ng/mL). The vertical blue (leftmost) and red (rightmost) lines denote the estimated mean (90% CI) ΔΔQTcF with plotted points at the geometric mean Cₘₐₓ of deuterated domperidone.

The same linear model was used for moxifloxacin. The slope of the relationship was positive and statistically significant. The lower bound of the 2-sided CI of the predicted QT effect at geometric mean peak moxifloxacin concentrations was >5 ms, demonstrating assay sensitivity.
(iii) A similar analysis to understand the relationship between plasma concentrations of the primary metabolite (M3) and ΔΔQTcF was also undertaken. The results of this analysis demonstrated that an effect on ΔΔQTcF exceeding 10 ms can be excluded up to M3 plasma concentrations of ˜480 ng/mL. The effect on ΔΔQTcF can be predicted to 1.43 ms (90% CI: 0.90 to 1.96) and 3.58 ms (90% CI: 2.34 to 4.83) for 30 and 100 mg treatment groups, respectively.

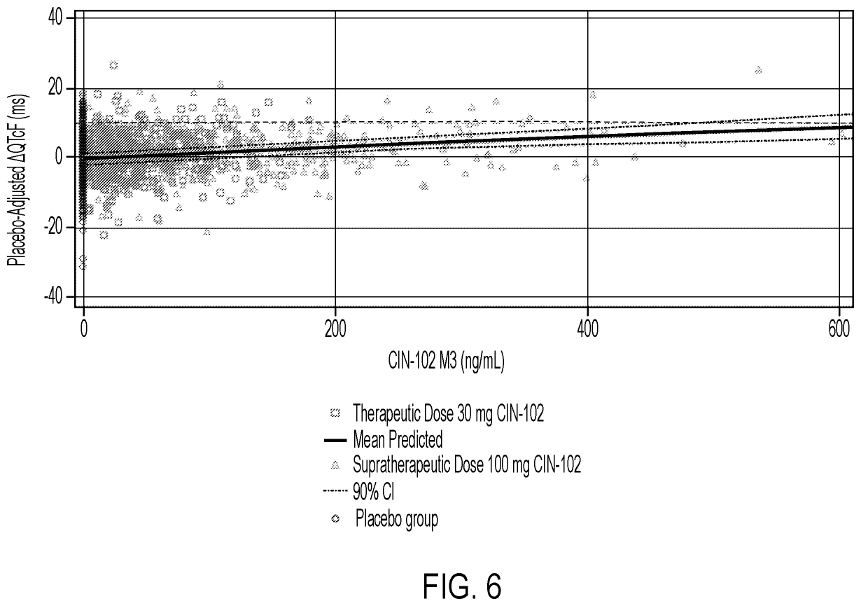

In FIG. 7, the solid black line with gray shaded area denotes the model-predicted mean (90% CI) ΔΔQTcF, which was calculated from the equation ΔΔQTcF=0.3678 (ms)+0.1439 (ms per ng/mL)×M3 concentration (ng/mL). Thus, the estimated population slope of the concentration-QTc relationship was very shallow: 0.14 ms per ng/mL (90% CI: 0.08 to 0.20) with a small treatment effect-specific intercept of 0.37 ms (90% CL: −0.16 to 0.89) (FIGS. 6 and 7).

The vertical blue (leftmost) and red (rightmost) lines denote the estimated mean (90% CI) ΔΔQTcF with plotted points at the geometric mean Cₘₐₓ of M3.
(iv) Deuterated domperidone at the studied doses did not have a clinically relevant effect on heart rate (FIG. 8) or cardiac conduction (i.e., the PR and QRS intervals).

Baseline ECG parameters were within expectations for a healthy population across treatment groups with mean HR between 59.9 and 61.0 bpm, mean QTcF between 404.0 and 406.1 ms, mean PR between 152.3 and 153.7 ms, and mean QRS between 103.0 and 103.7 ms.

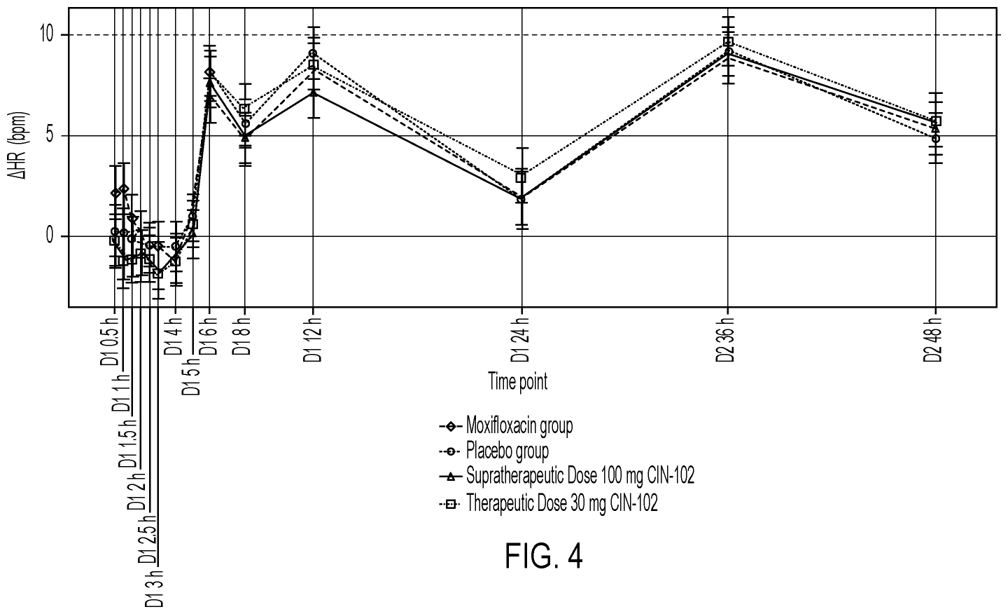

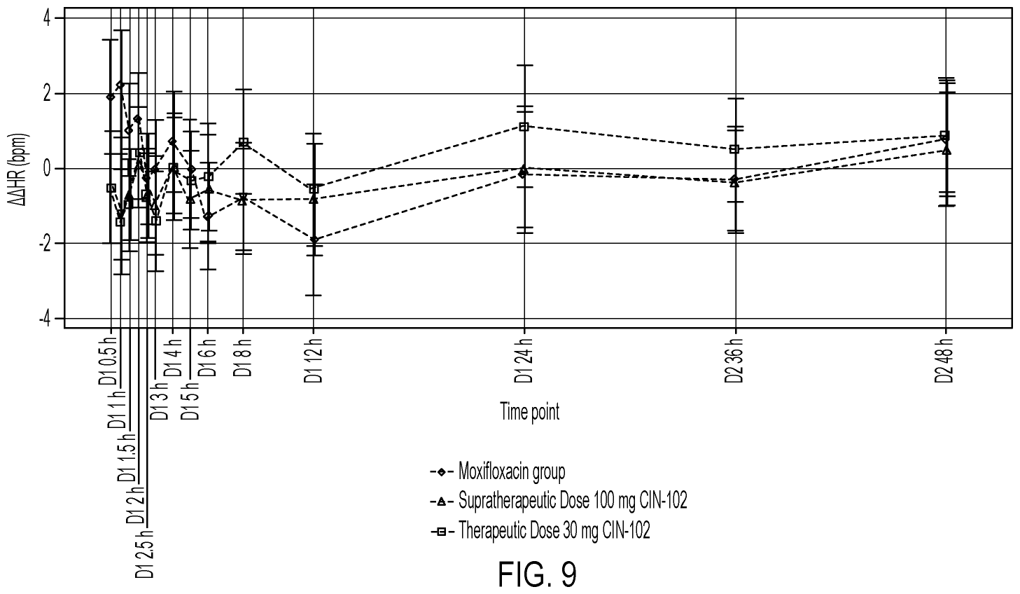

LS mean change-from-baseline HR (ΔHR) on CIN-102 closely followed the pattern observed on placebo (FIG. 4). LS mean placebo-corrected ΔHR (ΔΔHR) across post-dose time points during the CIN-102 treatment periods ranged within ±1.4 bpm, from −1.4 to 1.1 bpm (at 3 and 24 hours post-dose, respectively, both in the 30 mg treatment group) (FIG. 9 and Table 21). There was 1 bradycardic (at 5 time points in the 30 mg treatment group) and no tachycardic outliers across treatment periods.

TABLE 21
Placebo-corrected change-from-baseline HR (ΔΔHR)
at each time point (QT/QTc population)

| Parameter: ΔΔHR | | | | |
|---|---|---|---|---|
| Time point | Statistics | Treatment A | Treatment B | Treatment C |
| 0.5 H Post Dose | LS Mean | −0.5 | −0.5 | 1.9 |
| | SE | 0.91 | 0.91 | 0.91 |
| | 90% CI | (−2.00; 1.00) | (−2.03; 0.98) | (0.37; 3.40) |
| 1 H Post Dose | LS Mean | −1.4 | −1.1 | 2.2 |
| | SE | 0.85 | 0.86 | 0.86 |
| | 90% CI | (−2.84; −0.01) | (−2.47; 0.36) | (0.80; 3.65) |
| 1.5 H Post Dose | LS Mean | −1.0 | −0.7 | 1.0 |
| | SE | 0.74 | 0.75 | 0.75 |
| | 90% CI | (−2.21; 0.25) | (−1.94; 0.53) | (−0.22; 2.26) |
| 2 H Post Dose | LS Mean | 0.4 | 0.2 | 1.3 |
| | SE | 0.74 | 0.74 | 0.74 |
| | 90% CI | (−0.81; 1.63) | (−1.06; 1.39) | (0.08; 2.55) |
| 2.5 H Post Dose | LS Mean | −0.7 | −0.8 | −0.3 |
| | SE | 0.72 | 0.72 | 0.73 |
| | 90% CI | (−1.86; 0.53) | (−1.99; 0.41) | (−1.46; 0.95) |
| 3 H Post Dose | LS Mean | −1.4 | −1.0 | −0.0 |
| | SE | 0.80 | 0.80 | 0.80 |
| | 90% CI | (−2.74; −0.11) | (−2.30; 0.34) | (−1.36; 1.30) |
| 4 H Post Dose | LS Mean | 0.0 | 0.1 | 0.7 |
| | SE | 0.81 | 0.81 | 0.81 |
| | 90% CI | (−1.32; 1.35) | (−1.20; 1.48) | (−0.64; 2.05) |
| 5 H Post Dose | LS Mean | −0.3 | −0.8 | −0.0 |
| | SE | 0.78 | 0.78 | 0.79 |
| | 90% CI | (−1.62; 0.97) | (−2.12: 0.48) | (−1.34; 1.28) |
| 6 H Post Dose | LS Mean | −0.2 | −0.5 | −1.3 |
| | SE | 0.86 | 0.85 | 0.86 |
| | 90% CI | (−1.64; 1.19) | (−1.96; 0,87) | (−2.68; 0.16) |
| 8 H Post Dose | LS Mean | 0.7 | −0.9 | 0.7 |
| | SE | 0.85 | 0.86 | 0.86 |
| | 90% CI | (−0.69; 2.14) | (−2.30; 0.54) | (−2.17; 0.69) |
| 12 H Post Dose | LS Mean | −0.5 | −0.8 | −1.9 |
| | SE | 0.90 | 0.90 | 0.91 |
| | 90% CI | (−2.04; 0.95) | (−2.32; 0.67) | (−3.42; −0.41) |
| 24 H Post Dose | LS Mean | 1.1 | 0.1 | −0.1 |
| | SE | 0.98 | 0.98 | 0.98 |
| | 90% CI | (−0.52; 2.72) | (−1.56; 1.67) | (−1.74; 1.52) |
| 36 H Post Dose | LS Mean | 0.5 | −0.4 | −0.3 |
| | SE | 0.83 | 0.83 | 0.84 |
| | 90% CI | (−0.89; 1.86) | (−1.74; 1.02) | (−1.69; 1.08) |
| 48 H Post Dose | LS Mean | 0.9 | 0.5 | 0.8 |
| | SE | 0.91 | 0.91 | 0.91 |
| | 90% CI | (−0.62; 2.39) | (−1.01; 1.99) | (−0.72; 2.30) |

Based on a linear mixed-effects model for active and placebo treatment groups: ΔHR=Time +Treatment+TimexTreatment+Baseline HR+Period+Sequence. An unstructured covariance structure was used to specify the repeated measures at post-dose time points for subject within treatment period. The model also included a subject-specific random effect.

Treatment A: 30 mg CIN-102 [representing approximate steady-state therapeutic exposures in subject population] Treatment B: 100 mg CIN-102 [representing potential steady-state supratherapeutic exposures in subjects] Treatment C: 400 mg open-label moxifloxacin

The same linear model with a treatment effect-specific intercept as in the concentration-QTc analysis was used in the assay sensitivity analysis for moxifloxacin. The slope of the relationship was positive and statistically significant: 0.07 ms per ng/mL (90% CI: 0.06 to 0.09), and the lower bound of the 2-sided CI of the predicted QT effect (12.98 ms [90% CI: 11.43 to 14.52]) at the geometric mean peak moxifloxacin concentration (1552.6 ng/mL) was above 5 ms, thereby demonstrating assay sensitivity.

### Effect on Cardiac Repolarization: The QT Interval

### (a) By-Time Point (Central Tendency) and Categorical Analyses Results

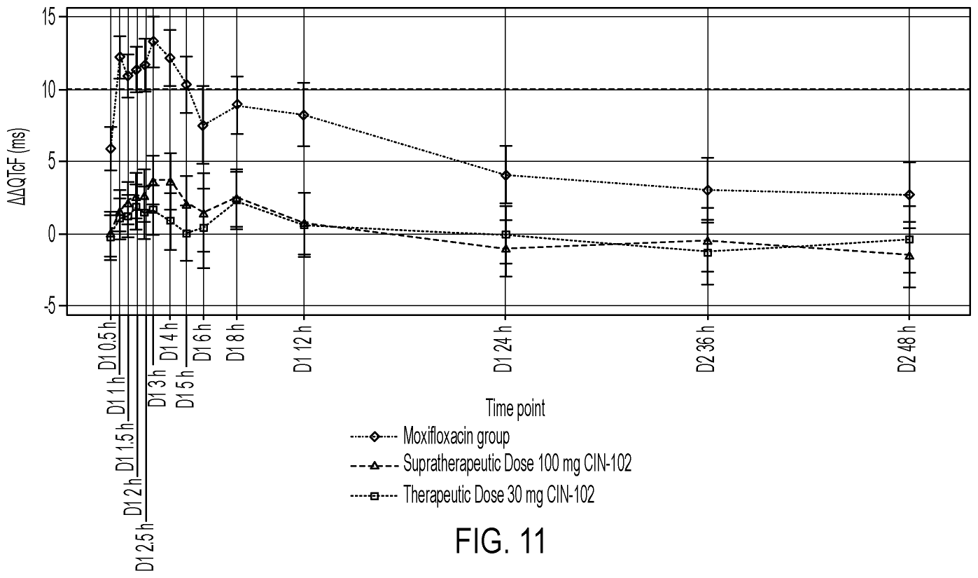

LS mean change-from-baseline QTcF (ΔQTcF) on CIN-102 followed the placebo pattern across post-dose time points with somewhat larger effect in the highest treatment group (100 mg CIN-102) between 3 and 6 hours post-dose (FIG. 5). LS mean placebo-corrected ΔQTcF (ΔΔQTcF) within the first 4 hours on CIN-102 ranged from −0.3 to 1.8 ms at 0.5 and 2 hour(s) post-dose, respectively, in the 30 mg treatment group and from −0.1 to 3.7 ms at 0.5 and 3 hour(s) post-dose, respectively, in the 100 mg treatment group (FIG. 11 and Table 22). The upper bound of the 90% CI of ΔΔQTcF was below 10 ms with both CIN-102 doses across all time points.

After dosing with 400 mg oral moxifloxacin, a clear increase of LS mean ΔΔQTcF was observed with a peak value of 13.3 ms (90% CI: 11.52 to 15.00) at 3 hours post-dose.

There were no subjects with QTcF >480 ms or ΔQTcF >30 ms (Tables 23 and 24). Across treatment groups, there were 6 and 1 subject(s) (at 19 and 1 time point[s]) with QTcF >450 and <480 ms during the moxifloxacin and placebo treatment periods, respectively. The following treatment-emergent T-wave morphology changes were observed: 2 subjects (at 2 time points) with flat T waves during the 100 mg treatment period; between 1 and 2 subjects (at between 1 and 7 time points) with biphasic T waves across all treatment periods; and, 1 subject each (at 10 and 1 time points) with inverted T waves during the 30 mg and moxifloxacin treatment periods, respectively). There were no treatment-emergent U waves.

TABLE 22
Placebo-corrected change-from-baseline QTcF (ΔΔQTcF)
at each time point QT/QTc population)

| Parameter: ΔΔQTcF | | | | |
|---|---|---|---|---|
| Time point | Statistics | Treatment A | Treatment B | Treatment C |
| 0.5 H Post Dose | LS Mean | −0.3 | −0.1 | 5.8 |
| | SE | 0.92 | 0.93 | 0.93 |
| | 90% CI | (−1.84; 1.22) | (−1.58; 1.48) | (4.31; 7.39) |
| 1 H Post Dose | LS Mean | 1.0 | 1.5 | 12.2 |
| | SE | 0.85 | 0.86 | 0.86 |
| | 90% CI | (−0.42; 2.41) | (0.12; 2.96) | (10.76; 13.62) |
| 1.5 H Post Dose | LS Mean | 1.2 | 2.1 | 10.9 |
| | SE | 0.88 | 0.89 | 0.89 |
| | 90% CI | (−0.28; 2.65) | (0.64; 3.58) | (9.41; 12.37) |
| 2 H Post Dose | LS Mean | 1.8 | 2.6 | 11.3 |
| | SE | 0.94 | 0.95 | 0.95 |
| | 90% CI | (0.23; 3.36) | (1.02; 4.16) | (9.76; 12.92) |
| 2.5 H Post Dose | LS Mean | 1.4 | 2.6 | 11.7 |
| | SE | 1.09 | 1.09 | 1.10 |
| | 90% CI | (−0.41; 3.20) | (0.80; 4.42) | (9.84; 13.48) |
| 3 H Post Dose | LS Mean | 1.6 | 3.7 | 13.3 |
| | SE | 1.04 | 1.05 | 1.05 |
| | 90% CI | (−0.12; 3.33) | (1.95; 5.41) | (11.52; 15.00) |
| 4 H Post Dose | LS Mean | 0.8 | 3.6 | 12.1 |
| | SE | 1.18 | 1.19 | 1.20 |
| | 90% CI | (−1.15; 2.77) | (1.63; 5.57) | (10.13; 14.09) |
| 5 H Post Dose | LS Mean | −0.0 | 2.0 | 10.3 |
| | SE | 1.16 | 1.16 | 1.17 |
| | 90% CI | (−1.94; 1.88) | (0.13; 3.96) | (8.35; 12.21) |
| 6 H Post Dose | LS Mean | 0.3 | 1.4 | 7.5 |
| | SE | 1.65 | 1.65 | 1.66 |
| | 90% CI | (−2.39; 3.08) | (−1.30; 4.16) | (4.73; 10.22) |
| 8 H Post Dose | LS Mean | 2.3 | 2.5 | 8.9 |
| | SE | 1.20 | 1.20 | 1.21 |
| | 90% CI | (0.29; 4.25) | (0.46; 4.45) | (6.86; 10.87) |
| 12 H Post Dose | LS Mean | 0.6 | 0.7 | 8.2 |
| | SE | 1.32 | 1.32 | 1.32 |
| | 90% CI | (−1.62; 2.74) | (−1.49; 2.87) | (5.99; 10.37) |
| 24 H Post Dose | LS Mean | −0.1 | −1.1 | 4.0 |
| | SE | 1.19 | 1.19 | 1.20 |
| | 90% CI | (−2.09; 1.85) | (−3.03; 0.91) | (2.02; 5.98) |
| 36 H Post Dose | LS Mean | −1.3 | −0.5 | 3.0 |
| | SE | 1.33 | 1.34 | 1.34 |
| | 90% CI | (−3.49; 0.93) | (−2.69; 1.74) | (0.79; 5.24) |
| 48 H Post Dose | LS Mean | −0.4 | −1.5 | 2.6 |
| | SE | 1.38 | 1.38 | 1.38 |
| | 90% CI | (−2.70; 1.86) | (−3.78; 0.77) | (0.35; 4.92) |

Based on a linear mixed-effects model for active and placebo treatment groups: ΔQTcF=Time+Treatment+Time×Treatment+Baseline QTcF+Period+Sequence. An unstructured covariance structure was used to specify the repeated measures at post-dose time points for subject within treatment period. The model also included a subject-specific random effect. Treatment A: 30 mg CIN-102 [representing approximate steady-state therapeutic exposures in subject population] Treatment B: 100 mg CIN-102 [representing potential steady-state supratherapeutic exposures in subjects] Treatment C: 400 mg open-label moxifloxacin.

TABLE 23
QTcF outliers per absolute category (QT/QTc population)

| | | Treat- | Treat- | Treat- | | Treat- |
|---|---|---|---|---|---|---|
| | Category | ment A | ment B | ment C | | ment D |
| Subject | Total | 52 | 52 | 51 | | 55 |
| | QTcF >450 | 0 | 0 | 6 | (11.8%) | 1 (1.8%) |
| | and <=480 ms | | | | | |
| | QTcF >480 | 0 | 0 | 0 | | 0 |
| | and <=500 ms | | | | | |
| | QTcF >500 ms | 0 | 0 | 0 | | 0 |
| Time | Total | 776 | 778 | 762 | | 825 |
| point | QTcF >450 | 0 | 0 | 19 | (2.5%) | 1 (0.1%) |

| | | | | |
|---|---|---|---|---|
| and <=480 ms | | | | |
| QTcF >480 | 0 | 0 | 0 | 0 |
| and <=500 ms | | | | |
| QTcF >500 ms | 0 | 0 | 0 | 0 |

Treatment A: 30 mg CIN-102 [representing approximate steady-state therapeutic exposures in subject population]; Treatment B: 100 mg CIN-102 [representing potential steady-state supratherapeutic exposures in subjects]; Treatment C: 400 mg open-label moxifloxacin; Treatment D: CIN-102 placebo. A subject can appear in more than 1 category. The denominator (Total) is defined as the number of subjects or time points in the QT/QTc population for each treatment.

TABLE 24
QTcF outliers per change-from-baseline
category (QT/QTc population)

| | | Treat- | Treat- | Treat- | Treat- |
|---|---|---|---|---|---|
| | Category | ment A | ment B | ment C | ment D |
| Subject | Total | 52 | 52 | 51 | 55 |
| | QTcF >30 and <=60 | 0 | 0 | 0 | 0 |
| | ms | | | | |
| | QTcF >60 ms | 0 | 0 | 0 | 0 |
| Time | Total | 776 | 778 | 762 | 825 |
| point | QTcF >30 and <=60 | 0 | 0 | 0 | 0 |
| | ms | | | | |
| | QTcF >60 ms | 0 | 0 | 0 | 0 |

Treatment A: 30 mg CIN-102 [representing approximate steady-state therapeutic exposures in subject population]; Treatment B: 100 mg CIN-102 [representing potential steady-state supratherapeutic exposures in subjects]; Treatment C: 400 mg open-label moxifloxacin; Treatment D: CIN-102 placebo. A subject can appear in more than 1 category. The denominator (Total) is defined as the number of subjects or time points in the QT/QTc population for each treatment.

### (b) Concentration-QTc Results

### Pharmacokinetic Profiles

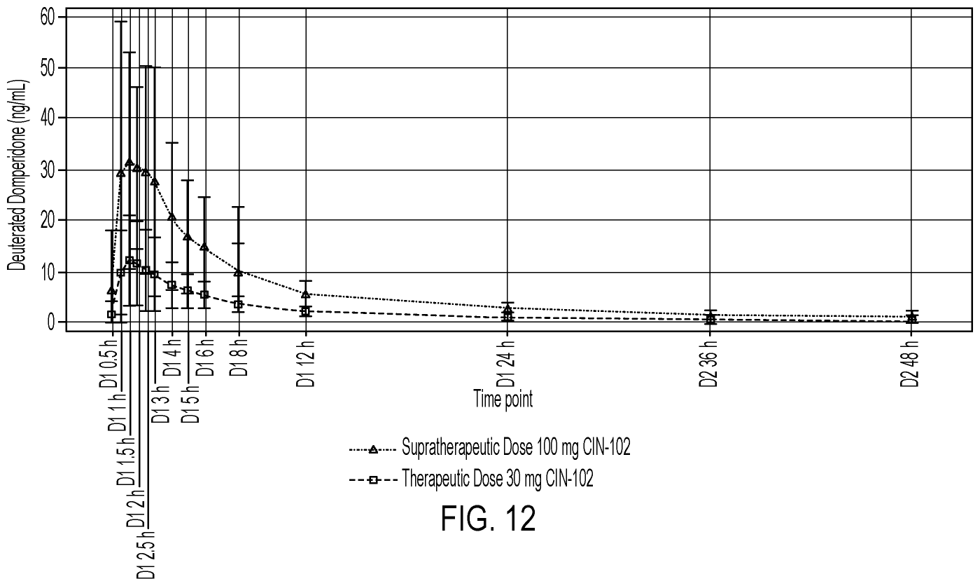

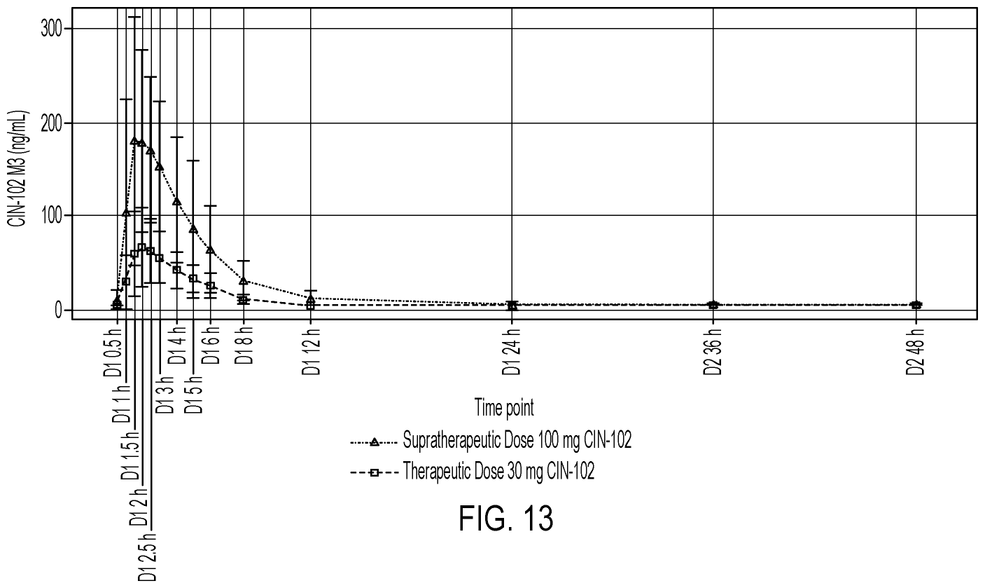

The time courses of deuterated domperidone plasma concentrations and its metabolite, M3, are shown in FIGS. 12 and 13. The highest mean plasma concentrations of deuterated domperidone were observed at 1.5 and 2 hours post-dose for the 30 and 100 mg treatment groups, respectively. The highest mean plasma concentrations of M3 were each observed at 2 hours post-dose for the 30 and 100 mg treatment groups.

### Assessment of Model-Independent Assumptions

The following model-independent checks of assumptions using exploratory plots were performed.

### Heart Rate Effect

In the by-time point analysis, mean ΔΔHR was below 10 bpm on both dose levels of CIN-102 across all post-dose time points (FIG. 9 and Table 21), thereby demonstrating that CIN-102 has no relevant effect on HR.

### Linear Concentration-QTc Relationship

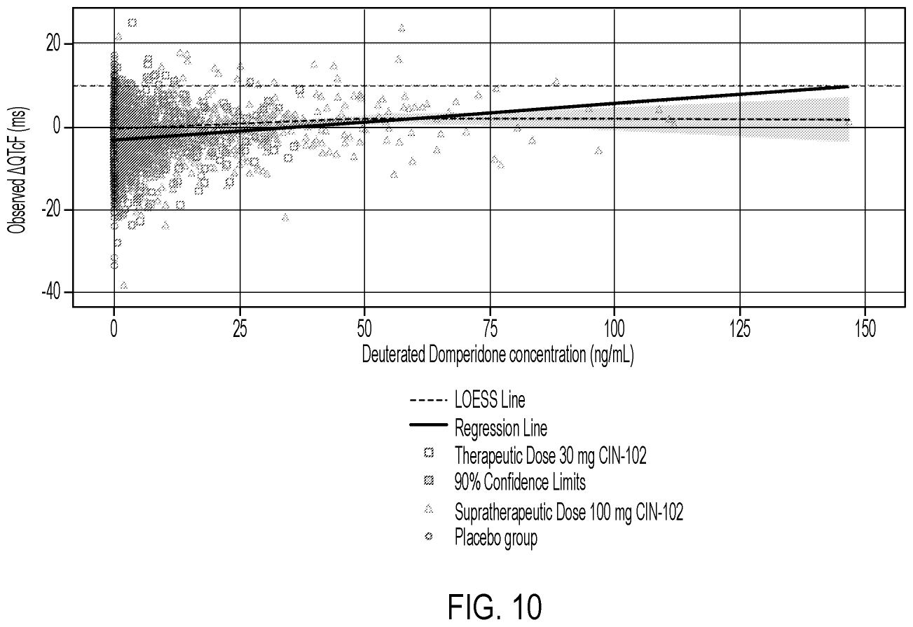

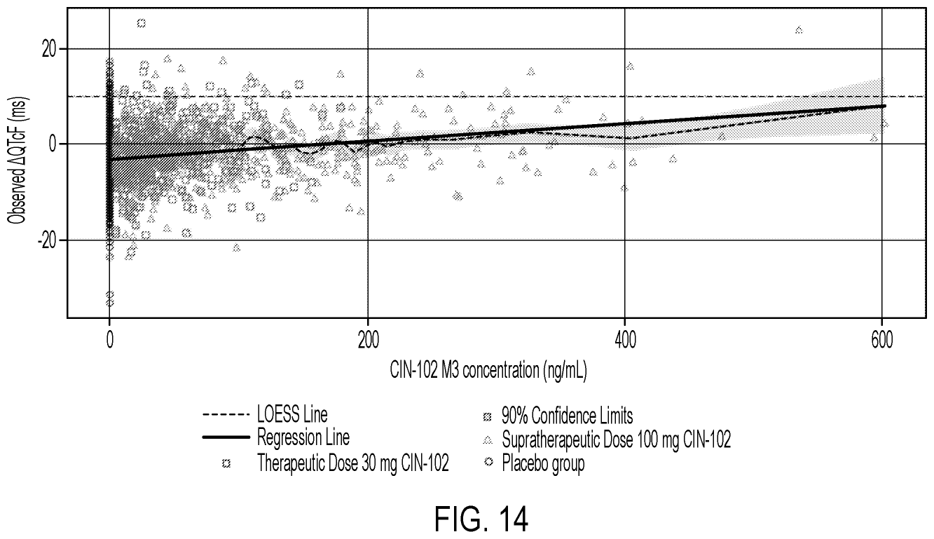

The scatter plots of concentrations of deuterated domperidone and M3 versus ΔQTcF with a linear regression line and a LOESS regression line (90% CI) are given in FIGS. 14 and 10. The linear regression lines and LOESS regression lines were close across all ranges of observed concentrations with minor deviations at the high concentrations for deuterated domperidone. This indicates that a linear model for the concentration-QTc relationship may appropriately capture the observed data.

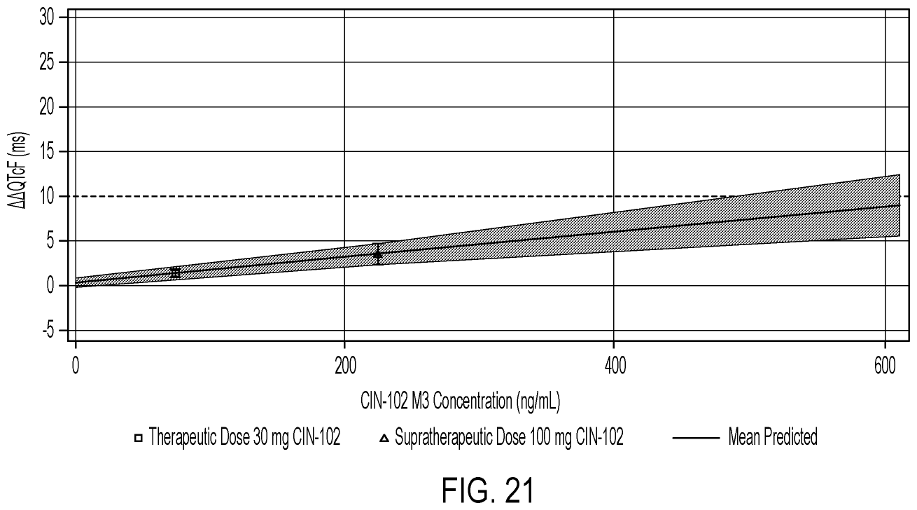

The predicted ΔΔQTcF effect using Model C at the geometric mean concentrations for M3 is shown in Table 25 and FIG. 21. An effect on ΔΔQTcF exceeding 10 ms can be excluded up to M3 plasma concentrations of ˜480 ng/mL.

TABLE 25
Predicted ΔΔQTcF interval at geometric mean peak CIN-102
M3 (ng/mL) concentration for Model C (PK/QTc population)

| Treatment | ΔΔQTcF Estimate (ms) (90% CI) |
|---|---|
| 30 mg CIN-102 | 1.43 (0.90, 1.96) |
| 100 mg CIN-102 | 3.58 (2.34, 4.83) |
| 10 ms Threshold | 7.28 (4.53, 10.02) |

Based on a linear mixed-effects model with ΔQTcF as the dependent variable, time-matched deuterated domperidone plasma concentration as an explanatory variate, centered baseline QTcF as an additional covariate, treatment (active=1 or placebo=0) and time as fixed effects, and a random intercept and slope per subject.

### The Concentration-QTc Model for Model B (Deuterated Domperidone)

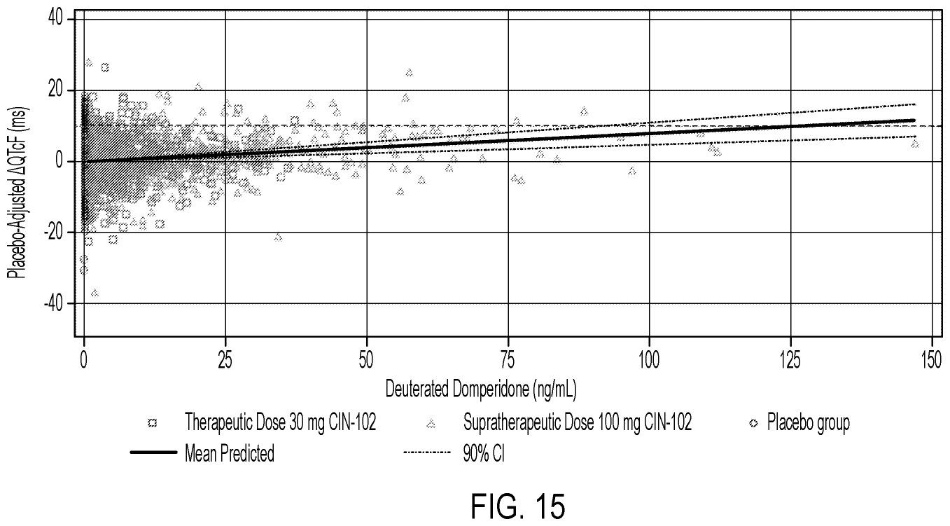

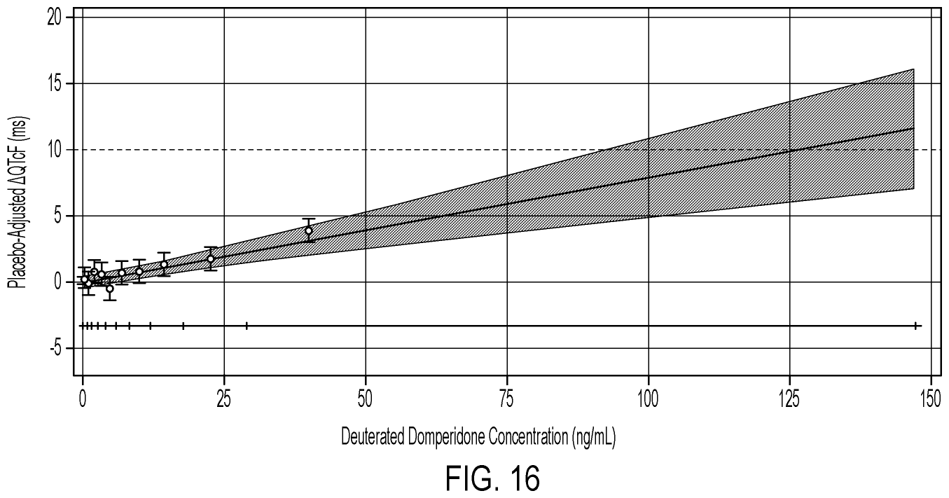

The relationship between the individually observed deuterated domperidone plasma concentrations and estimated placebo-adjusted ΔQTcF (i.e., ΔΔQTcF) is shown in FIG. 15.

The predicted ΔΔQTcF effect using Model B at the geometric mean concentrations for deuterated domperidone is shown in Table 26 and FIG. 8. An effect on ΔΔQTcF exceeding 10 ms can be excluded up to deuterated domperidone plasma concentrations of ˜92 ng/mL.

TABLE 26
Predicted ΔΔQTcF interval at geometric mean peak deuterated
domperidone concentration for Model B (PK/QTc population)

| Treatment | ΔΔQTcF Estimate (ms) (90% CI) | |
|---|---|---|
| 30 mg CIN-102 | 1.10 | (0.58, 1.61) |
| 100 mg CIN-102 | 3.50 | (2.18, 4.82) |
| 10 ms Threshold | 7.26 | (4.47, 10.04) |

Based on a linear mixed-effects model with ΔQTcF as the dependent variable, time-matched deuterated domperidone plasma concentration as an explanatory variate, centered baseline QTcF as an additional covariate, treatment (active=1 or placebo=0) and time as fixed effects, and a random intercept and slope per subject.

### Effect on Cardiac Conduction: The PR and QRS Intervals

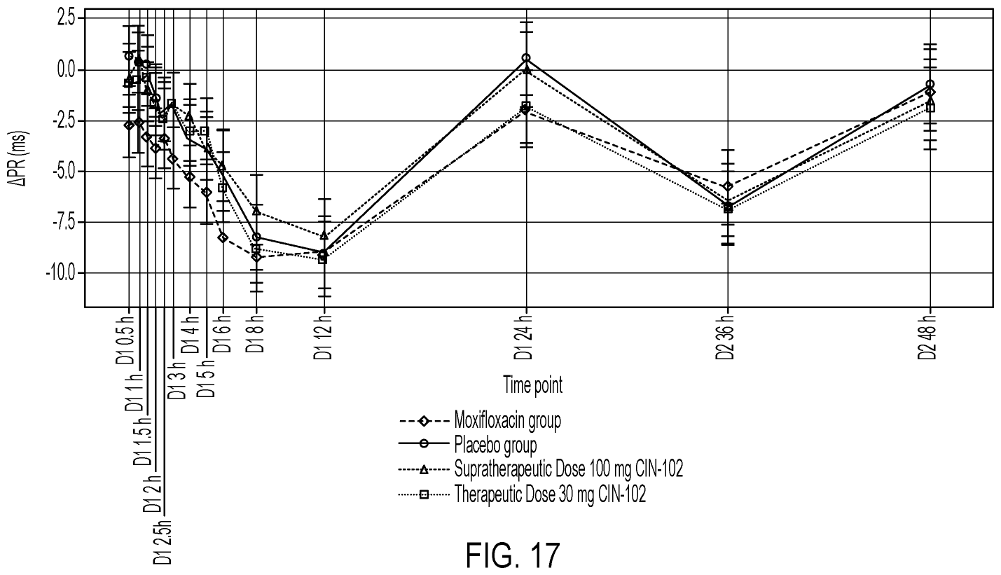

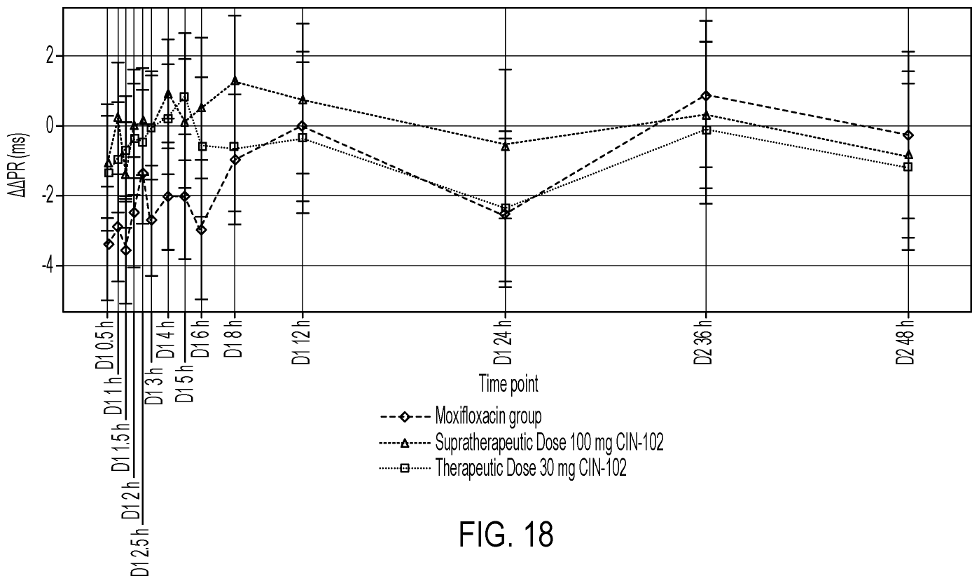

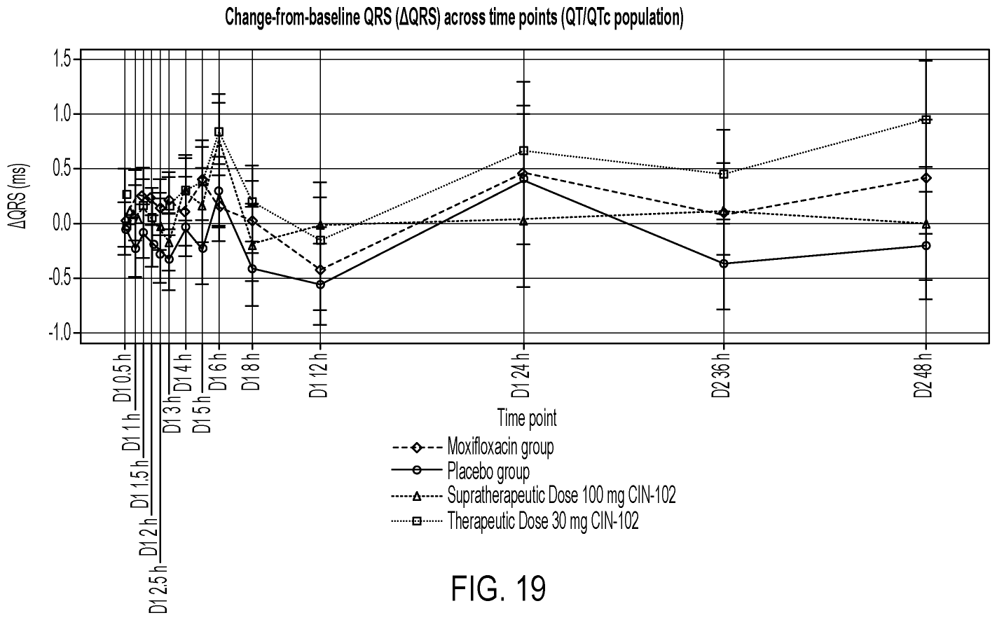

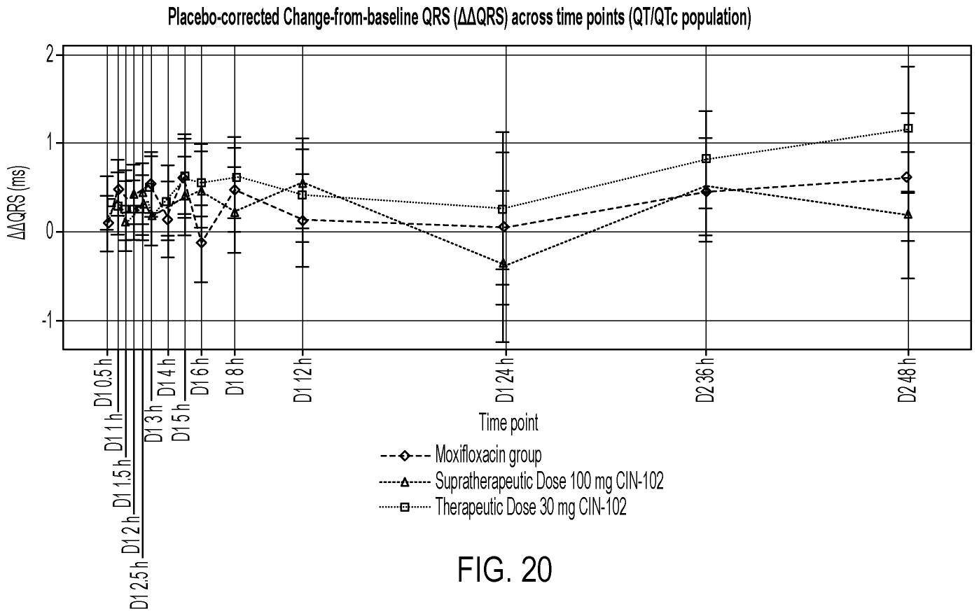

CIN-102 at the studied doses did not have a clinically relevant effect on cardiac conduction, i.e., the PR and QRS intervals. LS mean change-from-baseline PR (ΔPR) on CIN-102 closely followed the pattern observed in placebo subjects (FIG. 17). LS mean placebo-corrected ΔPR (ΔΔPR) on CIN-102 varied between ±2.3 ns, from −2.3 to 1.3 at 24 and 8 hours post-dose during the 30 and 100 mg treatment periods, respectively (FIG. 18 and Table 27). LS mean change-from-baseline QRS (ΔQRS) was small (FIG. 19) and LS mean placebo-corrected ΔQRS (ΔΔQRS) was within ±1.1 ms across all post-dose time points (FIG. 20 and Table 28). There were no PR or QRS outliers.

TABLE 27
Placebo-corrected change-from-baseline PR (ΔΔPR)
at each time point (QT/QTc population)

| Parameter: ΔΔPR | | | | |
|---|---|---|---|---|
| Time point | Statistics | Treatment A | Treatment B | Treatment C |
| 0.5 H Post Dose | LS Mean | −1.3 | −1.0 | −3.4 |
| | SE | 0.98 | 0.98 | 0.99 |
| | 90% CI | (−2.96; 0.28) | (−2.62; 0.63) | (−5.01; −1.74) |
| 1 H Post Dose | LS Mean | −0.9 | 0.3 | −2.9 |
| | SE | 0.95 | 0.95 | 0.96 |
| | 90% CI | (−2.45; 0.69) | (−1.30; 1.85) | (−4.48; −1.31) |
| 1.5 H Post Dose | LS Mean | −0.7 | −1.4 | −3.6 |
| | SE | 0.90 | 0.91 | 0.91 |
| | 90% CI | (−2.15; 0.84) | (−2.88; 0.13) | (−5.07; −2.04) |
| 2 H Post Dose | LS Mean | −0.4 | 0.1 | −2.5 |
| | SE | 0.94 | 0.94 | 0.95 |
| | 90% CI | (−1.92; 1.19) | (−1.50; 1.63) | (−4.05; −0.90) |
| 2.5 H Post Dose | LS Mean | −0.4 | 0.2 | −1.3 |
| | SE | 0.89 | 0.89 | 0.90 |
| | 90% CI | (−1.90; 1.04) | (−1.32; 1.64) | (−2.81; 0.17) |
| 3 H Post Dose | LS Mean | −0.1 | −0.0 | −2.7 |
| | SE | 0.94 | 0.94 | 0.95 |
| | 90% CI | (−1.61; 1.50) | (−1.57; 1.56) | (−4.29; −1.14) |
| 4 H Post Dose | LS Mean | 0.2 | 1.0 | −2.0 |
| | SE | 0.93 | 0.94 | 0.94 |
| | 90% CI | (−1.33; 1.76) | (−0.58; 2.53) | (−3.58; −0.45) |
| 5 H Post Dose | LS Mean | 0.9 | 0.1 | −2.0 |
| | SE | 1.09 | 1.10 | 1.11 |
| | 90% CI | (−0.95; 2.67) | (−1.72; 1,90) | (−3.86; −0.20) |
| 6 H Post Dose | LS Mean | −0.6 | 0.5 | −3.0 |
| | SE | 1.21 | 1.21 | 1.22 |
| | 90% CI | (−2.58; 1.44) | (−1.47; 2.55) | (−4.99; −0.95) |
| 8 H Post Dose | LS Mean | −0.6 | 1.3 | −0.9 |
| | SE | 1.12 | 1.13 | 1.13 |
| | 90% CI | (−2.46; 1.25) | (−0.54; 3.19) | (−2.83; 0.93) |
| 12 H Post Dose | LS Mean | −0.3 | 0.8 | −0.0 |
| | SE | 1.29 | 1.29 | 1.30 |
| | 90% CI | (−2.47; 1.81) | (−1.33; 2.95) | (−2.15; 2.15) |
| 24 H Post Dose | LS Mean | −2.3 | −0.5 | −2.5 |
| | SE | 1.29 | 1.28 | 1.29 |
| | 90% CI | (−4.47; −0.21) | (−2.65; 1.60) | (−4.66; −0.39) |
| 36 H Post Dose | LS Mean | −0.1 | 0.3 | 0.9 |
| | SE | 1.26 | 1.27 | 1.27 |
| | 90% CI | (−2.19; 2.00) | (−1.76; 2.43) | (−1.17; 3.05) |
| 48 H Post Dose | LS Mean | −1.1 | −0.8 | −0.3 |
| | SE | 1.44 | 1.44 | 1.45 |
| | 90% CI | (−3.53; 1.24) | (−3.17; 1.59) | (−2.68; 2.11) |

Based on a linear mixed-effects model for active and placebo treatment groups: ΔPR=Time +Treatment+Time×Treatment+Baseline PR Period Sequence. An unstructured covariance structure was used to specify the repeated measures at post-dose time points for subject within treatment period. The model also included a subject-specific random effect. Treatment A: 30 mg CIN-102 [representing approximate steady-state therapeutic exposures in subject population]; Treatment B: 100 mg CIN-102 [representing potential steady-state supratherapeutic exposures in subjects]; Treatment C: 400 mg open-label moxifloxacin

TABLE 28
Placebo-corrected change-from-baseline QRS (ΔΔQRS)
at each time point (QT/QTc population)

| Parameter: ΔΔQRS | | | | |
|---|---|---|---|---|
| Time point | Statistics | Treatment A | Treatment B | Treatment C |
| 0.5 H Post Dose | LS Mean | 0.3 | 0.1 | 0.1 |
| | SE | 0.19 | 0.19 | 0.19 |
| | 90% CI | (−0.00; 0.63) | (−0.24; 0.39) | (−0.21; 0.42) |
| 1 H Post Dose | LS Mean | 0.3 | 0.3 | 0.5 |
| | SE | 0.19 | 0.19 | 0.19 |
| | 90% CI | (−0.04; 0.58) | (0.03; 0.65) | (0.18; 0.81) |
| 1.5 H Post Dose | LS Mean | 0.2 | 0.1 | 0.3 |
| | SE | 0.21 | 0.21 | 0.21 |
| | 90% CI | (−0.09; 0.59) | (−0.23; 0.45) | (−0.00; 0.68) |
| 2 H Post Dose | LS Mean | 0.2 | 0.2 | 0.4 |
| | SE | 0.21 | 0.21 | 0.21 |
| | 90% CI | (−0.10; 0.59) | (−0.12; 0.58) | (0.07; 0.77) |
| 2.5 H Post Dose | LS Mean | 0.2 | 0.3 | 0.4 |
| | SE | 0.21 | 0.21 | 0.21 |
| | 90% CI | (−0.11; 0.60) | (−0.06; 0.65) | (0.08; 0.79) |
| 3 H Post Dose | LS Mean | 0.5 | 0.2 | 0.6 |
| | SE | 0.21 | 0.21 | 0.21 |
| | 90% CI | (0.16; 0.84) | (−0.16; 0.52) | (0.21; 0.90) |
| 4 H Post Dose | LS Mean | 0.3 | 0.3 | 0.1 |
| | SE | 0.25 | 0.25 | 0.25 |
| | 90% CI | (−0.06; 0.75) | (−0.10; 0.72) | (−0.28; 0.54) |
| 5 H Post Dose | LS Mean | 0.6 | 0.4 | 0.7 |
| | SE | 0.27 | 0.27 | 0.27 |
| | 90% CI | (0.16; 1.06) | (−0.05; 0.85) | (0.20; 1.11) |
| 6 H Post Dose | LS Mean | 0.6 | 0.5 | −0.1 |
| | SE | 0.26 | 0.26 | 0.26 |
| | 90% CI | (0.12; 0.99) | (0.04; 0.90) | (−0.57; 0.30) |
| 8 H Post Dose | LS Mean | 0.6 | 0.2 | 0.5 |
| | SE | 0.28 | 0.28 | 0.29 |
| | 90% CI | (0.14; 1.08) | (−0.23; 0.70) | (−0.01; 0.94) |
| 12 H Post Dose | LS Mean | 0.4 | 0.5 | 0.1 |
| | SE | 0.32 | 0.32 | 0.32 |
| | 90% CI | (−0.11; 0.94) | (0.02; 1.07) | (−0.39; 0.66) |
| 24 H Post Dose | LS Mean | 0.3 | −0.4 | 0.0 |
| | SE | 0.52 | 0.52 | 0.52 |
| | 90% CI | (−0.60; 1.12) | (−1.24; 0.47) | (−0.82; 0.90) |
| 36 H Post Dose | LS Mean | 0.8 | 0.5 | 0.5 |
| | SE | 0.33 | 0.33 | 0.34 |
| | 90% CI | (0.27; 1.37) | (−0.05; 1.06) | (−0.10; 1.01) |
| 48 H Post Dose | LS Mean | 1.1 | 0.2 | 0.6 |
| | SE | 0.43 | 0.43 | 0.43 |
| | 90% CI | (0.43; 1.87) | (−0.52; 0.91) | (−0.10; 1.33) |

Based on a linear mixed-effects model for active and placebo treatment groups: ΔQRS=Time+Treatment+Time×Treatment+Baseline QRS+Period+Sequence. An unstructured covariance structure was used to specify the repeated measures at post-dose time points for subject within treatment period. The model also included a subject-specific random effect. Treatment A: 30 mg CIN-102 [representing approximate steady-state therapeutic exposures in subject population]; Treatment B: 100 mg CIN-102 [representing potential steady-state supratherapeutic exposures in subjects]; Treatment C: 400 mg open-label moxifloxacin.

### Discussion and Conclusion

This was a randomized, partially double-blind placebo- and active-controlled, single dose, four-period, crossover study to assess the effect of CIN-102 on cardiac repolarization in healthy subjects. The primary objective of this study was to assess the effect of CIN-102 on cardiac repolarization based on baseline and placebo-corrected change in QTcF. Twelve lead ECGs, paired with PK sampling, were extracted at 3 time points prior to dosing and post-dose in each period.

At baseline, data were available from all 55 subjects in the placebo treatment period and then from 52 subjects in the CIN-102 therapeutic (30 mg) and supratherapeutic (100 mg) treatment periods and from 51 subjects in the moxifloxacin treatment period.

CIN-102 at the studied doses did not have a clinically relevant effect on heart rate or on cardiac conduction, i.e., the PR and QRS intervals.

The highest mean plasma concentrations of deuterated domperidone were observed at 1.5 and 2 hours post-dose for the 30 and 100 mg treatment groups, respectively. The highest mean plasma concentrations of M3 were each observed at 2 hours post-dose for the 30 and 100 mg treatment groups.

LS mean ΔΔQTcF was very small on both CIN-102 doses and varied within a narrow range during the first 4 hours post-dose: between −0.3 and 1.8 ms at 0.5 and 2 hour(s) post-dose, respectively, in the 30 mg treatment group and between −0.1 and 3.7 ms at 0.5 and 3 hour(s) post-dose, respectively, in the 100 mg treatment group. Notably, the upper bound of the 90% CI of

ΔΔQTcF was below 10 ms with both CIN-102 doses across all time points, i.e., thereby demonstrating that an effect on ΔΔQTcF exceeding 10 ms could be excluded on both CIN-102 doses.

In the concentration-QTc analysis, a full model including deuterated domperidone and its metabolites, M3, was initially fitted and a model selection procedure was performed. The model with M3 alone (Model C) was selected as primary due to smallest AIC value and represented the data in an acceptable way. The estimated slope of M3 plasma concentration in the concentration-QTc relationship was positive and statistically significant (0.14 ms per ng/mL [90% CI: 0.08 to 0.20]) with a small, not statistically significant treatment effect-specific intercept of 0.37 ms. The effect on ΔΔQTcF can be predicted to 1.43 ms (90% CI: 0.90 to 1.96) and 3.58 ms (90% CI: 2.34 to 4.83) for 30 and 100 mg treatment groups, respectively.

The results from the concentration-QTc model with deuterated domperidone alone (Model B) were similar to those from the primary model. The estimated slope of deuterated domperidone plasma concentration in the concentration-QTc relationship was positive and statistically significant (0.79 ms per ng/mL [90% CI: 0.47 to 1.11]) with a small, not statistically significant treatment effect-specific intercept of −0.03 ms. The effect on ΔΔQTcF can be predicted to 1.10 ms (90% CI: 0.58 to 1.61) and 3.50 ms (90% CI: 2.18 to 4.82) for 30 and 100 mg treatment groups, respectively.

Based on this concentration-QTc analysis, a QTcF effect (ΔΔQTcF) exceeding 10 ms can be excluded up to deuterated domperidone and M3 plasma concentrations of ˜92 and 480 ng/mL, respectively.

The same linear model with a treatment effect-specific intercept as in the concentration-QTc analysis was used in the assay sensitivity analysis for moxifloxacin. The slope of the relationship was positive and statistically significant: 0.07 ms per ng/mL (90% CI: 0.06 to 0.09), and the lower bound of the 2-sided CI of the predicted QT effect (12.98 ms [90% CI: 11.43 to 14.52]) at the geometric mean peak moxifloxacin concentration (1552.6 ng/mL) was above 5 ms, thereby demonstrating assay sensitivity.

In summary, CIN-102 at the studied doses had no clinically relevant effects on studied ECG parameters. Based on the concentration-QTc analysis, an effect on ΔΔQTcF exceeding 10 ms can be excluded up to deuterated domperidone and M3 plasma concentrations of ˜92 and 480 ng/mL, respectively.

In conclusion, the results of this negative TQT study indicate that small QT effects can be excluded at concentrations of deudomperidone and M3 approximately 5-fold higher than that produced by the current top therapeutic dose in patients with gastroparesis (10 mg BID) which has a mean Ca of deuterated domperidone of approximately 18.2 ng/mL. There were no clinically relevant effects of CIN-102 on heart rate or cardiac conduction. No deaths or serious adverse events occurred during the study. Deuterated domperidone at therapeutic and supratherapeutic doses does not have a clinically meaningful effect on ECG parameters, including the QT interval.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made, and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

1. A method for increasing gastric emptying in a human having gastroparesis, comprising orally administering 10 mg to 120 mg of deudomperidone daily to the human.

2. The method of claim 1, wherein the gastroparesis is idiopathic gastroparesis or diabetic gastroparesis.

3. The method of claim 1, wherein the human is experiencing an upper gastrointestinal symptom prior to the administration.

4. The method of claim 3, wherein the upper gastrointestinal symptom is postprandial nausea, postprandial vomiting, postprandial fullness, early satiety, bloating, epigastric pain, abdominal pain, or combinations thereof.

5. (canceled)

6. The method of claim 1, wherein the increase in gastric emptying is measured by the gastric emptying breath test (GEBT) about 12 to about 14 days after administering the deudomperidone, as measured by a gastric emptying breath test (GEBT) using ¹³C-Spirulina platensis.

7. The method of claim 1, wherein the human has a delayed gastric emptying about 10 days prior to first dose of deudomperidone, as measured by a gastric emptying breath test (GEBT) using ¹³C-Spirulina platensis.

8. The method of claim 1, wherein, prior to administering the deudomperidone, the human has:
ii) a T½ greater than about 80 minutes, or such as greater than about 200 minutes, as measured by a gastric emptying breath test (GEBT) using ¹³C-Spirulina platensis,
(ii) an American Neurogastroenterology and Motility Society Gastroparesis Cardinal Symptom Index Daily Diary (ANMS GCSI-DD) average composite score of about 2 or greater;
(iii) a ANMS GCSI-DD nausea subscale score of about 2 or greater for at least about 4 days:
(iv) a combination of (i)-(iii).

9. (canceled)

10. (canceled)

11. The method of claim 1, wherein about 10 mg, or about 20 mg, or about or about 30 mg, or about 60 mg, or about 120 mg of deudomperidone is administered to the human.

12. The method of claim 1, wherein the deudomperidone is administered in divided doses, such as two doses.

13. The method of claim 1, wherein the human is an adult.

14. The method of claim 1, wherein administration results in a clinically significant improvement in nausea, early satiety, postprandial fullness, upper abdominal pain, vomiting, and bloating as measured by an ANMS GCSI-DD score after the treatment.

15. The method of claim 1, wherein the administration results in a clinically significant improvement in an average composite score of the ANMS GCSI-DD score after the treatment.

16. The method of claim 1, wherein the administration results in a clinically significant improvement in a nausea subscale score of the ANMS GCSI-DD score after the treatment.

17. The method of claim 1, wherein the administration results in a clinically significant improvement in gastric emptying as measured by (i) GEBT after about 12 to about 16 days after the treatment, (ii) ANMS GCSI-DD total scores after the treatment, (iii) ANMS GCSI-DD subscale scores after the treatment, or (iv) a combination of (i)-(iii).

18. (canceled)

19. (canceled)

20. The method of claim 1, wherein the human has diabetic neuropathy.

21. The method of claim 1, wherein, prior to administering the deudomperidone, the human has a T½ greater than about 110 minutes.

22. The method of claim 1, wherein, prior to administering the deudomperidone, the human has a T½ less than about 110 minutes.

23. The method of claim 1, wherein the deudomperidone administration does not cause a clinically significant QT prolongation.

24. The method of claim 1, wherein the administration does not result in a clinically significant change in the heart rate or cardiac conduction of the human.

25. (canceled)

26. The method of claim 1, wherein the pulse rate (PR) interval of the human is about 0.12 to about 0.2 seconds, the QRS interval of the human is about 0.08 and 0.10 seconds, or a combination thereof after administration of the deudomperidone.

27. (canceled)

28. The method of claim 1, wherein the administration results in a ΔΔQTcF that is less than about 10 ms.

29. The method of claim 28, wherein the amount of deudomperidone is about 30 mg and the administration results in a ΔΔQTcF of about 1.1 ms or less.

30. The method of claim 29, wherein the amount of deudomperidone is about 100 mg and the administration results in a ΔΔQTcF of about 3.5 ms or less.
